(19) United States
(12) Patent Application Publication
(10) Pub. No.: US 2024/0002879 A1
(43) Pub. Date: Jan. 4, 2024

(54) GORILLA ADENOVIRUS NUCLEIC ACID- AND AMINO ACID-SEQUENCES, VECTORS CONTAINING SAME, AND USES THEREOF

(71) Applicant: REITHERA SRL, Rome (IT)

(72) Inventors: Stefano Colloca, Rome (IT); Armin Lahm, Rome (IT); Angelo Raggioli, Pomezia (IT); Alessandra Vitelli, Rome (IT)

(21) Appl. No.: 18/003,858

(22) PCT Filed: Jul. 1, 2021
(86) PCT No.: PCT/EP2021/068124

§ 371 Date: Dec. 29, 2022

(30) Foreign Application Priority Data
Jul. 1, 2020 (EP) 20183515.4

(51) Int. Cl. C12N 15/86; C07K 14/005; A61K 39/215; A61P 31/14

(52) U.S. Cl. CPC C12N 15/86; C07K 14/005; A61K 39/215; A61P 31/14; C12N 2770/20022; C12N 2710/10022; C12N 2710/10343; C12N 2710/10351; C12N 2710/10371; C12N 2710/10023; A61K 2039/5258

(57) ABSTRACT

The present invention relates to novel adenovirus strains with a high immunogenicity and very low pre-existing immunity in the general human population. The absence of detectable neutralizing antibodies is due to novel hypervariable regions in the adenoviral capsid protein hexon. The present invention provides nucleotide and amino acid sequences of these novel adenovirus strains, as well as recombinant viruses, virus-like particles and vectors based on these strains. Further provided are pharmaceutical compositions and medical uses in the therapy or prophylaxis of a disease, and methods for producing an adenovirus or virus-like particles utilizing the novel sequences, recombinant viruses, virus-like particles and vectors.

### CROSS REFERENCE TO RELATED APPLICATIONS

The present application is a U.S. national phase application under 35 U.S.C. § 371 of International Application No. PCT/EP2021/068124, filed on Jul. 1, 2021 and published as WO 2022/003083 A1 on Jan. 6, 2022, the entire content of which is incorporated herein by reference in its entirety.

### INCORPORATION BY REFERENCE OF MATERIAL IN ASCII TEXT FILE

A Sequence Listing contained in the following ASCII text file being submitted concurrently herewith: File Name: ZSP007001APC_ST25.txt; created Feb. 22, 2023, which is 2,342,623 bytes in size. This Sequence Listing in electronic format is hereby expressly incorporated by reference in its entirety.

The present invention relates to novel adenovirus strains with a high immunogenicity and very low pre-existing immunity in the general human population. The absence of detectable neutralizing antibodies is due to novel hypervariable regions in the adenoviral capsid protein hexon. The present invention provides nucleotide and amino acid sequences of these novel adenovirus strains, as well as recombinant viruses, virus-like particles and vectors based on these strains. Further provided are pharmaceutical compositions and medical uses in the therapy or prophylaxis of a disease, and methods for producing an adenovirus or virus-like particles utilizing the novel sequences, recombinant viruses, virus-like particles and vectors.

### BACKGROUND OF THE INVENTION

The adenoviruses (Ads) comprise a large family of double-stranded DNA viruses found in amphibians, avians, and mammals which have a nonenveloped icosahedral capsid structure (Straus, Adenovirus infections in humans; The Adenoviruses, 451-498, 1984; Hierholzer et al., J. Infect. Dis., 158: 804-813, 1988; Schnurr and Dondero, Intervirology., 36: 79-83, 1993; Jong et al., J. Clin. Microbiol., 37: 3940-3945: 1999). In contrast to retroviruses, adenoviruses can transduce numerous cell types of several mammalian species, including both dividing and non-dividing cells, without integrating into the genome of the host cell.

Generally speaking, adenoviral DNA is typically very stable and remains episomal (e.g. extrachromosomal), unless transformation or tumorigenesis occurs. In addition, adenoviral vectors can be propagated to high yields in well-defined production systems which are readily amenable to pharmaceutical scale production of clinical grade compositions. These characteristics and their well-characterized molecular genetics make recombinant adenoviral vectors good candidates for use as vaccine carriers. The production of recombinant adenoviral vectors may rely on the use of a packaging cell line which is capable of complementing the functions of adenoviral gene products that have been either deleted or engineered to be non-functional.

Presently, two well-characterized human subgroup C adenovirus serotypes (i.e., hAd2 and hAd5) are widely used as the sources of the viral backbone for most of the adenoviral vectors that are used for gene therapy. Replication-defective human adenoviral vectors have also been tested as vaccine carriers for the delivery of a variety of immunogens derived from a variety of infectious agents. Studies conducted in experimental animals (e. g. rodents, canines and nonhuman primates) indicate that recombinant replication-defective human adenoviral vectors carrying transgenes encoding immunogens as well as other antigens elicit both humoral and cell-mediated immune responses against the transgene product. Generally speaking, investigators have reported success using human adenoviral vectors as vaccine carriers in non human experimental systems by either using immunization protocols that utilizes high doses of recombinant adenoviral vectors that are predicted to elicit immune responses; or by using immunization protocols which employ the sequential administration of adenoviral vectors that are derived from different serotypes but which carry the same transgene product as boosting immunizations (Mastrangeli, et. al., Human Gene Therapy, 7: 79-87 (1996)).

Vectors derived from species C adenoviruses (e.g. Ad5, Ad6 and ChAd3) are the most immunogenic (Colloca et al., Sci. Transl. Med. 4 (115), 2012). In particular, viral vectors based on human adenovirus type 5 (Ad5) have been developed for gene therapy and vaccine applications. Although Ad5-based vectors are extremely efficient in animal models, the presence of pre-existing neutralizing antibodies in humans against Ad5 wild type virus (in particular directed to the capsid as shown in FIG. 1) has in clinical trials been demonstrated to reduce the efficiency of gene transduction (Moore J P et al. Science. 2008 May 9; 320(5877):753-5). Such antibodies are largely directed to the hypervariable regions of the hexon protein. Immunity in the general population limits the broad application of Ad vectored-vaccines based on Ad5. On the other hand, rare human adenoviruses are less immunogenic than Ad5 (Colloca et al., Sci. Transl. Med. 4 (115), 2012). Vectors based on non-human adenoviruses have a very low pre-existing immunity in the general human population (Farina et al., J. Virol. 75 (23), 11603-11613, 2001). Some non-human adenovirus vectors are known, but since immunity against these can develop in humans, there is an ongoing need for further adenovirus vectors with high immunogenicity and a low or absent pre-existing neutralizing antibodies in humans.

### SUMMARY OF THE INVENTION

In a first aspect, the invention provides a polynucleotide encoding an adenovirus hexon protein comprising:
A) (i) a HVR1 comprising an amino acid sequence according to position 136 to 168 of SEQ ID NO: 2, or a variant thereof comprising up to two mutations,
   (ii) a HVR2 comprising an amino acid sequence according to position 187 to 201 of SEQ ID NO: 2, or a variant thereof comprising up to two mutations,
   (iii) a HVR3 comprising an amino acid sequence according to position 219 to 225 of SEQ ID NO: 2, or a variant thereof comprising up to two mutations,
   (iv) a HVR4 comprising an amino acid sequence according to position 257 to 268 of SEQ ID NO: 2, or a variant thereof comprising up to two mutations,
   (v) a HVR5 comprising an amino acid sequence according to position 276 to 290 of SEQ ID NO: 2, or a variant thereof comprising up to two mutations,
   (vi) a HVR6 comprising an amino acid sequence according to position 314 to 322 Y of SEQ ID NO: 2, or a variant thereof comprising up to two mutations, and
   (vii) a HVR7 comprising an amino acid sequence according to position 431 to 456 of SEQ ID NO: 2, or a variant thereof comprising up to two mutations; or
B) (i) a HVR1 comprising an amino acid sequence according to position 136 to 168 of SEQ ID NO: 9, or a variant thereof comprising up to two mutations,
   (ii) a HVR2 comprising an amino acid sequence according to position 187 to 201 of SEQ ID NO: 9, or a variant thereof comprising up to two mutations,
   (iii) a HVR3 comprising an amino acid sequence according to position 219 to 225 of SEQ ID NO: 9, or a variant thereof comprising up to two mutations,
   (iv) a HVR4 comprising an amino acid sequence according to position 257 to 268 of SEQ ID NO: 9, or a variant thereof comprising up to two mutations,
   (v) a HVR5 comprising an amino acid sequence according to position 276 to 290 of SEQ ID NO: 9, or a variant thereof comprising up to two mutations,
   (vi) a HVR6 comprising an amino acid sequence according to position 314 to 322 of SEQ ID NO: 9, or a variant thereof comprising up to two mutations, and
   (vii) a HVR7 comprising an amino acid sequence according to position 431 to 456 of SEQ ID NO: 9, or a variant thereof comprising up to two mutations; or
C) (i) a HVR1 comprising an amino acid sequence according to position 136 to 163 of SEQ ID NO: 11, or a variant thereof comprising up to two mutations,
   (ii) a HVR2 comprising an amino acid sequence according to position 182 to 196 of SEQ ID NO: 11, or a variant thereof comprising up to two mutations,
   (iii) a HVR3 comprising an amino acid sequence according to position 214 to 220 of SEQ ID NO: 11, or a variant thereof comprising up to two mutations,
   (iv) a HVR4 comprising an amino acid sequence according to position 252 to 262 of SEQ ID NO: 11, or a variant thereof comprising up to two mutations,
   (v) a HVR5 comprising an amino acid sequence according to position 270 to 278 of SEQ ID NO: 11, or a variant thereof comprising up to two mutations,
   (vi) a HVR6 comprising an amino acid sequence according to position 302 to 310 of SEQ ID NO: 11, or a variant thereof comprising up to two mutations, and
   (vii) a HVR7 comprising an amino acid sequence according to position 419 to 442 of SEQ ID NO: 11, or a variant thereof comprising up to two mutations; or
D) (i) a HVR1 comprising an amino acid sequence according to position 136 to 168 of SEQ ID NO: 17, or a variant thereof comprising up to two mutations,
   (ii) a HVR2 comprising an amino acid sequence according to position 187 to 201 of SEQ ID NO: 17, or a variant thereof comprising up to two mutations,
   (iii) a HVR3 comprising an amino acid sequence according to position 219 to 225 of SEQ ID NO: 17, or a variant thereof comprising up to two mutations,
   (iv) a HVR4 comprising an amino acid sequence according to position 257 to 267 of SEQ ID NO: 17, or a variant thereof comprising up to two mutations,
   (v) a HVR5 comprising an amino acid sequence according to position 275 to 289 of SEQ ID NO: 17, or a variant thereof comprising up to two mutations,
   (vi) a HVR6 comprising an amino acid sequence according to position 313 to 321 of SEQ ID NO: 17, or a variant thereof comprising up to two mutations, and
   (vii) a HVR7 comprising an amino acid sequence according to position 430 to 455 of SEQ ID NO: 17, or a variant thereof comprising up to two mutations; or
E) (i) a HVR1 comprising an amino acid sequence according to position 136 to 168 of SEQ ID NO: 19, or a variant thereof comprising up to two mutations,
   (ii) a HVR2 comprising an amino acid sequence according to position 187 to 201 of SEQ ID NO: 19, or a variant thereof comprising up to two mutations,
   (iii) a HVR3 comprising an amino acid sequence according to position 219 to 225 of SEQ ID NO: 19, or a variant thereof comprising up to two mutations,
   (iv) a HVR4 comprising an amino acid sequence according to position 257 to 268 of SEQ ID NO: 19, or a variant thereof comprising up to two mutations,
   (v) a HVR5 comprising an amino acid sequence according to position 276 to 290 of SEQ ID NO: 19, or a variant thereof comprising up to two mutations,
   (vi) a HVR6 comprising an amino acid sequence according to position 314 to 322 of SEQ ID NO: 19, or a variant thereof comprising up to two mutations, and
   (vii) a HVR7 comprising an amino acid sequence according to position 431 to 456 of SEQ ID NO: 19, or a variant thereof comprising up to two mutations; or
F) (i) a HVR1 comprising an amino acid sequence according to position 136 to 168 of SEQ ID NO: 21, or a variant thereof comprising up to two mutations,
   (ii) a HVR2 comprising an amino acid sequence according to position 187 to 201 of SEQ ID NO: 21, or a variant thereof comprising up to two mutations,
   (iii) a HVR3 comprising an amino acid sequence according to position 219 to 225 of SEQ ID NO: 21, or a variant thereof comprising up to two mutations,
   (iv) a HVR4 comprising an amino acid sequence according to position 257 to 267 of SEQ ID NO: 21, or a variant thereof comprising up to two mutations,
   (v) a HVR5 comprising an amino acid sequence according to position 275 to 289 of SEQ ID NO: 21, or a variant thereof comprising up to two mutations,
   (vi) a HVR6 comprising an amino acid sequence according to position 313 to 321 of SEQ ID NO: 21, or a variant thereof comprising up to two mutations, and
   (vii) a HVR7 comprising an amino acid sequence according to position 430 to 455 of SEQ ID NO: 21, or a variant thereof comprising up to two mutations; or
G) (i) a HVR1 comprising an amino acid sequence according to position 136 to 168 of SEQ ID NO: 23, or a variant thereof comprising up to two mutations,
   (ii) a HVR2 comprising an amino acid sequence according to position 187 to 201 of SEQ ID NO: 23, or a variant thereof comprising up to two mutations,
   (iii) a HVR3 comprising an amino acid sequence according to position 219 to 225 of SEQ ID NO: 23, or a variant thereof comprising up to two mutations,
   (iv) a HVR4 comprising an amino acid sequence according to position 257 to 268 of SEQ ID NO: 23, or a variant thereof comprising up to two mutations,
   (v) a HVR5 comprising an amino acid sequence according to position 276 to 290 of SEQ ID NO: 23, or a variant thereof comprising up to two mutations,
   (vi) a HVR6 comprising an amino acid sequence according to position 314 to 322 of SEQ ID NO: 23, or a variant thereof comprising up to two mutations, and
   (vii) a HVR7 comprising an amino acid sequence according to position 431 to 456 of SEQ ID NO: 23, or a variant thereof comprising up to two mutations; wherein the polynucleotide encoding an adenovirus hexon protein according to G) further encodes for an adenovirus fiber protein according to SEQ ID NO: 6, or a variant thereof comprising up to two mutations.

In a second aspect, the invention provides a hexon polypeptide encoded by the polynucleotide as defined in A), B), C), D), E) or F) of the first aspect.

In a third aspect, the invention provides an adenoviral capsid comprising hexon, fiber and penton proteins, wherein for A)-F) the hexon is the hexon encoded by the polynucleotide of the first aspect, and for G) the hexon and the fiber are the hexon and fiber encoded by the polynucleotide of the first aspect.

In a fourth aspect, the invention provides an adenovirus (i) encoded by an polynucleotide of the first aspect, (ii) comprising a polynucleotide according to the first aspect and/or (iii) comprising a hexon polypeptide of the second aspect or the capsid of the third aspect.

In a fifth aspect, the invention provides a virus-like particle (i) encoded by a polynucleotide of the first aspect and/or (ii) comprising a hexon polypeptide of the second aspect or the capsid of the third aspect.

In a sixth aspect, the invention provides a vector comprising a polynucleotide of the first aspect.

In a seventh aspect, the invention provides a composition comprising (i) an adjuvant, (ii) a polynucleotide of the first aspect, a hexon polypeptide of the second aspect, an adenoviral capsid polypeptide of the third aspect, an adenovirus of the fourth aspect, a virus-like particle of the fifth aspect, or a vector of the sixth aspect, and optionally (iii) a pharmaceutically acceptable excipient.

In an eighth aspect, the invention provides a cell comprising a polynucleotide of the first aspect, a hexon polypeptide of the second aspect, an adenoviral capsid polypeptide of the third aspect, an adenovirus of the fourth aspect, a virus-like particle of the fifth aspect, or a vector of the sixth aspect.

In a ninth aspect, the invention provides a polynucleotide of the first aspect, a hexon polypeptide of the second aspect, an adenoviral capsid polypeptide of the third aspect, an adenovirus of the fourth aspect, a virus-like particle of the fifth aspect, a vector of the sixth aspect, a composition of the seventh aspect and/or a cell of the eighth aspect for use in treating or preventing a disease.

In a tenth aspect, the invention relates to an in vitro method for producing an adenovirus or an adenovirus-like particle, comprising the steps of
(i) expressing a polynucleotide of the first aspect in a cell such that an adenovirus or an adenovirus-like particle is assembled in the cell,
(ii) isolating the adenovirus or the adenovirus-like particle from the cell or the medium surrounding the cell.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland) and as described in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

Throughout this specification and the claims which follow, unless the context requires otherwise, the word “comprise”, and variations such as “comprises” and “comprising”, will be understood to imply the inclusion of a stated feature, integer or step or group of features, integers or steps but not the exclusion of any other feature, integer or step or group of integers or steps. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Several documents are cited throughout the text of this specification. Each of the documents cited herein (including all patents, patent applications, scientific publications, manufacturer's specifications, instructions, etc.), whether supra or infra, are hereby incorporated by reference in their entirety.

### LEGENDS TO THE FIGURES

FIG. 1: Structure of the adenovirus capsid.

FIG. 2: Schematic representation shuttle plasmid.

FIG. 3: Schematic representation pGRAd23 DE1 GAG BAC.

FIG. 4: Schematic representation pGRAd23 DE1 GAG DE3 BAC.

FIG. 5: Schematic representation pGRAd23 DE1 GAG DE3 DE4 hAd5 E4orf6 BAC.

FIG. 6: T-cell response on mouse splenocytes. Each dot represents the response in a single mouse, and the line corresponds to the mean for each dose group. Injected dose in number of virus particles are shown on the x axis.

FIG. 7: Humoral response against GRAd23 DE1 encoding the Gag antigen. Each dot represents the response in a single mouse, and the line corresponds to the mean for each dose group.

FIG. 8: Seroprevalence of the GRAd23 vector on a panel of human sera. Single dots represent single serum sample (y axis neutralization titer). The table reports the % of sera that are negative (<18), with intermediate neutralization titer (<200) or with high titer (>200). FIG. 9: Seroprevalence of the GRAd32 vector on a panel of human sera. Single dots represent single serum sample, neutralization titer as vertical axis.

FIG. 10: Humoral response against GRAd21 DE1 encoding the Gag antigen. Each dot represents the response in a single mouse, and the line corresponds to the mean for each dose group.

FIG. 11: Spike antigen expression using GRAd32 DE1 encoding the SARS-COV2 Spike antigen.

FIG. 12: Spike antigen immunogenicity (ELIspot) using GRAd32 DE1 encoding the SARS-COV2 Spike antigen.

FIG. 13: Anti-spike serum antibody responses following immunization with GRAd32 DE1 encoding the SARS-COV2 Spike antigen.

FIG. 14: Spike-2P expression. HeLa cells were infected with 50 MOI of the indicated vectors. 48 h after infection cell lysates were collected and analyzed by SDS-PAGE Western Blot. In the top panel, membranes were blotted with an anti-HA antibody, recognizing the spike-2P protein (HA tag). In the bottom panel, GAPDH is used as loading control. 1: Mock, 2: GRAd23b-S2P, 3: GRAd33b-S2P, 4: GRAd34b-S2P, 2: GRAd39b-S2P.

FIG. 15: Immunogenicity as measured by antibody endpoint titers of GRAd vectors two (w2) or five (w5) weeks following immunization of BALB/c mice with the indicated dose (viral particles). Numbers below each group of datapoints indicate the geometric mean.

FIG. 16: SARS-CoV-2 specific binding and neutralizing antibody responses in GRAd-COV2 vaccinated volunteers. Antibody response to SARS-CoV-2 induced by GRAd-COV2 vaccination at low dose (LD-5×10\hat{ }10 vp-circles), intermediate dose (ID-1×10\hat{ }11 vp-upright triangles) and high dose (HD-2×10\hat{ }11 vp-upside down triangles). 18-55 y and 65-85 y identify younger and older age cohorts, respectively. Horizontal black lines within each group of datapoints are set at median across all panels. HCS: human convalescent sera (diamonds), obtained from either previously hospitalized (hosp-dark grey) or from non-hospitalized (non-hosp-light grey) COVID-19 patients, and the NIB SC 20/130 standard plasma (filled circle) are shown for reference. (A) IgG binding to S1-S2 measured by CLIA at the day of vaccination (d0), and 1, 2 or 4 weeks after vaccination. Data are expressed as arbitrary unit (AU)/ml. Filled and dashed lines are set at 12 and 15 AU/ml. According to manufacturer, results >15 are clearly positive, between 12 and 15 are equivocal and <12 are negative or may indicate low level of IgG antibodies to the pathogen. (B-C) SARS-CoV-2 specific IgG titers in sera collected at d0 and w4 post vaccination measured by ELISA on recombinant full-length Spike (B) or RBD (C). Data are expressed as endpoint titer, and for negative sera where a titer cannot be calculated, an arbitrary value of 50 (or one-half the first serum dilution tested) is assigned. (D-E) SARS-CoV-2 neutralizing antibodies at week 4 post vaccination detected by SARS-CoV-2 microneutralization assay (D), or by plaque reduction neutralization test (E). SARS-CoV-2 neutralization titers are expressed as MNA₉₀ and PRNT₅₀, or the reciprocal of serum dilution achieving 90% or 50% neutralization, respectively. Dashed lines indicate LOD, and negative sera are assigned a value of one/half the LOD.

FIG. 17: T cell response to Spike peptides, induced by GRAd-COV2 vaccination at low (LD-5×10\hat{ }10 vp-circles), intermediate dose (ID-1×10\hat{ }11 vp-upright triangles) and high dose (HD-2×10\hat{ }11 vp-upside down triangles). 18-55 y and 65-85 y labels identify younger and older age cohorts, respectively. Horizontal black lines are set at median across all panels. (A-B) IFNγ ELISpot on freshly isolated PBMC at w2. Data are expressed as IFN-γ spot forming cells (SFC)/106 PBMC. In (A) individual data points represent cumulative Spike T cell response, calculated by summing the response to each S1a, S1b, S2a and S2b peptide pool stimulation and correcting for background (DMSO stimulation) in each volunteer. HCP: freshly isolated human convalescent PBMC, obtained from subjects recovering from symptomatic SARS-CoV-2 infection. (B) distribution of IFNγ ELISpot response to individual Spike peptide pools. Dashed line indicates assay positivity cut off (48 SFC/million PBMC). (C-D-E-F) IFNγ/IL2/IL4/IL17 intracellular staining and FACS analysis at w2 on fresh PBMC of younger (C-D) and older volunteers (E-F). Data are expressed as the percentage of Spike-specific CD4 (C-E) or CD8 (D-F) secreting each cytokine (or for Any Th1 a combination of cytokines, i.e. sum of CD4 secreting IFNγ alone, IL2 alone, and both IFNγ and IL2), obtained by summing responses to each of the 4 Spike peptide pools and corrected for background (DMSO stimulation). The tables below C-E (CD4 graphs) show P values derived by Kruskall-Wallis test comparing Th1 any, IFN-g and IL-2 profiles with the IL-4 and IL-17 profiles within each dose group.

NUCLEOTIDE AND AMINO ACID SEQUENCES

The following Tables 1a and 1b provide an overview over the GRAds and the sequences referred to herein (GRAd+number: isolated adenoviral strain; *: corresponding nucleotide sequence of the GRAd genome encoding the amino acid sequence. GRAd (Gorilla Adenovirus) is the inventors' strain designation. The extent of the genomic coordinates for the hexon, penton, fiber given below does not include the final stop codon, which is optionally included/added in this disclosure when referring to a polynucleotide encoding hexon, penton or fiber using the coordinates.

TABLE 1a
GRAds with SEQ ID NOs referred to in the application

| | | | SEQ ID | Embodiment |
|---|---|---|---|---|
| GRAd | Polypeptide | Polynucleotide | NO | designation |
| 32 | | genome GRAd32 | 1 | A |
| | Hexon | *18988 . . . 21870 of SEQ ID NO: 1 | 2 | A |
| | Fiber | *32228 . . . 33976 of SEQ ID NO: 1 | 3 | A |
| | Penton | *14025 . . . 15977 of SEQ ID NO: 1 | 4 | A |
| 36 | | genome GRAd36 | 5 | A |
| | Hexon | *19003 . . . 21885 of SEQ ID NO: 5 | 2 | A |
| | Fiber | *32243 . . . 33991 of SEQ ID NO: 5 | 6 | A |
| | Penton | *14025 . . . 15992 of SEQ ID NO: 5 | 7 | A |
| 38 | | genome GRAd38 | 8 | B |
| | Hexon | *19003 . . . 21885 of SEQ ID NO: 8 | 9 | B |
| | Fiber | *32243 . . . 33991 of SEQ ID NO: 8 | 6 | B |
| | Penton | *14025 . . . 15992 of SEQ ID NO: 8 | 7 | B |
| 21 | | genome GRAd21 | 10 | C |
| | Hexon | *18878 . . . 21718 of SEQ ID NO: 10 | 11 | C |
| | Fiber | *32099 . . . 33838 of SEQ ID NO: 10 | 12 | C |
| | Penton | *14022 . . . 15872 of SEQ ID NO: 10 | 13 | C |
| 37 | | genome GRAd37 | 14 | C |
| | Hexon | *18878 . . . 21718 of SEQ ID NO: 14 | 11 | C |
| | Fiber | *32099 . . . 33838 of SEQ ID NO: 14 | 15 | C |
| | Penton | *14022 . . . 15872 of SEQ ID NO: 14 | 13 | C |
| 33 | | genome GRAd33 | 16 | D |
| | Hexon | *19003 . . . 21882 of SEQ ID NO: 16 | 17 | D |
| | Fiber | *32240 . . . 33988 of SEQ ID NO: 16 | 6 | D |
| | Penton | *14025 . . . 15992 of SEQ ID NO: 16 | 7 | D |
| 35 | | genome GRAd35 | 18 | E |
| | Hexon | *19003 . . . 21885 of SEQ ID NO: 18 | 19 | E |
| | Fiber | *32243 . . . 33991 of SEQ ID NO: 18 | 6 | E |
| | Penton | *14025 . . . 15992 of SEQ ID NO: 18 | 7 | E |
| 34 | | genome GRAd34 | 20 | F |
| | Hexon | *19003 . . . 21882 of SEQ ID NO: 20 | 21 | F |
| | Fiber | *32240 . . . 33988 of SEQ ID NO: 20 | 6 | F |
| | Penton | *14025 . . . 15992 of SEQ ID NO: 20 | 7 | F |
| 23 | | genome GRAd23 | 22 | G |
| | Hexon | *19003 . . . 21885 of SEQ ID NO: 22 | 23 | G |
| | Fiber | *32243 . . . 33991 of SEQ ID NO: 22 | 6 | G |
| | Penton | *14025 . . . 15992 of SEQ ID NO: 22 | 7 | G |

TABLE 1b
SEQ ID NOs referred to in the application

| SEQ ID NO | Polypeptide | Polynucleotide |
|---|---|---|
| 1-23 | See Table 1a | |
| 24 | SARS CoV-2 Spike protein | |
| | variant (Asp614Gly) | |
| 25 | SARS CoV-2 Spike protein | |
| | variant (Lys986Pro, Val987Pro) | |
| 26 | | pGRAd32 DE1 ALS DE3 DE4 |
| | | hAd5E4orf6 |
| 27 | | pGRAd23 DE1L A/L/S DE3 BAC |
| 28 | | pGRAd21 DE1 ALS DE3 DE4 |
| | | hAd5E4orf6 |
| 29 | | SARS CoV-2 surface glycoprotein S |
| | | (codon optimized/Kozak/HA TAG) |
| 30 | SARS CoV-2 Spike protein | *6 . . . 3824 SEQ ID NO: 29 |
| 31 | | pGRAd32 DE1 SARS-COV2 DE3 |
| | | DE4 |
| 32 | | pGRAd23 DE1L hCMVtetO- |
| | | IntronA::SARS CoV-2 S-WPRE- |
| | | bGHpA DE3 |
| 33 | | pGRAd21 DE1 SARS-COV2 DE3 |
| | | DE4 |
| 34 | | pUC57-GRAd ends |
| 35 | | BeloBAC11 |
| 36 | | pGRAd ITRs-only shuttle |
| 37 | | phCMVtetO-GAG-bGHpolyA |
| 38 | | pGRAd pIX |
| 39 | | pAmpR-LacZ-SacB |
| 40 | | pDE1 GRAd shuttle |
| 41 | | pGRAd23 DE1 GAG BAC |
| 42 | | pGRAd23 DE1 A/L/S BAC |
| 43 | | pGRAd23 DE1L GAG BAC |
| 44 | | pGRAd23 DE1 GAG DE3 A/L/S |
| | | BAC |
| 45 | | pGRAd23 DE1 GAG DE3 BAC |
| 46 | | pGRAd23 DE1 GAG DE4 A/L/S |
| | | BAC |
| 47 | | wild type human Adenovirus 5 |
| 48 | | pGRAd23 DE1 DE4 hAd5E4orf6 |
| | | BAC |
| 49 | | pGRAd23 DE1 GAG DE3 A/L/S DE4 |
| | | hAd5 E4orf6 BAC |
| 50 | | pGRAd23 DE1 GAG DE3 DE4 hAd5 |
| | | E4orf6 BAC |
| 51 | | pUC19-hCMVtetO::SEAP-bGHpA |
| 52 | | pVIJnsA |
| 53 | | pCAG21 |
| 54 | | phCMVtetO-IntronA::I-SceI-WPRE- |
| | | bGHpA |
| 55 | | phCMVtetO-IntronA::SARS CoV-2 |
| | | S-WPRE-bGHpA |
| 56 | | pGRAd23 DE1L GAG DE3 A/L/S |
| | | BAC |
| 57 | | pGRAd23 DE1L GAG DE3 BAC |
| 58 | | pGRAd32 DE1 GAG wrongITR-L |
| 59 | | pGRAd32 DE1 GAG wrongITR-L |
| | | ALS in ITR-L |
| 60 | | pGRAd32 DE1 GAG - ITRs corrected |
| 61 | | pGRAd32 DE1 GAG DE3 ALS |
| 62 | | pGRAd32 DE1 GAG DE3 |
| 63 | | pGRAd32 DE1 GAG DE3 DE4 ALS |
| 64 | | pGRAd32 DE1 GAG DE3 DE4 |
| | | hAd5E4orf6 |
| 65 | | pGRAd21 DE1 GAG |
| 66 | | pGRAd21 DE1 GAG DE3 ALS |
| 67 | | pGRAd21 DE1 GAG DE3 |
| 68 | | pGRAd21 DE1 GAG DE3 DE4 ALS |
| 69 | | pGRAd21 DE1 GAG DE3 DE4 |
| | | hAd5E4orf6 |
| 70-117 | | Oligonucleotides used for GRAd |
| | | construction |

The following Tables 2a, 2b, 2c, 2d and 2e provide the genomic boundaries/coordinates of CDSs, RNAs and ITRs in the genomes. They apply to any reference to genomic elements herein that are listed in these tables and are incorporated as preferred into the respective embodiments.

TABLE 2a
Genomic boundaries of CDSs, RNAs and ITRs for GRAd32 and GRAd23. E3_CR1-alpha denotes
a putative open-reading frame having a GTG as initial codon. rc denotes reverse complement.
Products generated by splicing are indicated by multiple coordinate pairs.

| ORF | GRAd32 (SEQ ID NO: 1) | GRAd23 (SEQ ID NO: 22) |
|---|---|---|
| E1A | (546 . . . 1059, 1167 . . . 1456) | (546 . . . 1059, 1167 . . . 1456) |
| E1B_SmallT_19K | (1657 . . . 2211) | (1657 . . . 2211) |
| E1B_LargeT_55K | (1962 . . . 3473) | (1962 . . . 3473) |
| E1B_IX | (3567 . . . 3965) | (3567 . . . 3965) |
| E2A_DBP | rc (22621 . . . 24264) | rc (22636 . . . 24279) |
| E2B_IVa2 | rc (4027 . . . 5357, 5636 . . . 5648) | rc (4027 . . . 5357, 5636 . . . 5648) |
| E2B_Polymerase | rc (5130 . . . 8708, 13980 . . . 13988) | rc (5130 . . . 8708, 13980 . . . 13988) |
| E2B_pTP | rc (8510 . . . 10465, 13980 . . . 13988) | rc (8510 . . . 10465, 13980 . . . 13988) |
| L1_52-55KD | (10919 . . . 12142) | (10919 . . . 12142) |
| L1_IIIa | (12171 . . . 13955) | (12171 . . . 13955) |
| L2_Penton | (14025 . . . 15977) | (14025 . . . 15992) |
| L2_VII | (16009 . . . 16614) | (16024 . . . 16629) |
| L2_V | (16687 . . . 17769) | (16702 . . . 17784) |
| L2_X | (17801 . . . 18031) | (17816 . . . 18046) |
| L3_VI | (18132 . . . 18881) | (18147 . . . 18896) |
| L3_Hexon | (18988 . . . 21870) | (19003 . . . 21885) |
| L3_Endoprotease | (21895 . . . 22524) | (21910 . . . 22539) |
| L4_100kD | (24309 . . . 26804) | (24324 . . . 26819) |
| L4_22kD | (26488 . . . 27084) | (26503 . . . 27099) |
| L4_33kD | (26488 . . . 26833, 27111 . . . 27394) | (26503 . . . 26848, 27126 . . . 27409) |
| L4_VIII | (27455 . . . 28135) | (27470 . . . 28150) |
| E3_12.5K | (28139 . . . 28459) | (28154 . . . 28474) |
| E3_CR1-alpha | (28440 . . . 28985) | (28455 . . . 29000) |
| E3_gp19K | (29183 . . . 29662) | (29198 . . . 29677) |
| E3_CR1-beta | (29710 . . . 30576) | (29725 . . . 30591) |
| E3_CR1-gamma | (30622 . . . 30933) | (30637 . . . 30948) |
| E3_RID-alpha | (30945 . . . 31214) | (30960 . . . 31229) |
| E3_RID-beta | (31221 . . . 31640) | (31236 . . . 31655) |
| E3_14.7K | (31636 . . . 32019) | (31651 . . . 32034) |
| L5_Fiber | (32171 . . . 33976) | (32186 . . . 33991) |
| E4_Orf6-7 | rc (34167 . . . 34442, 35145 . . . 35327) | rc (34182 . . . 34457, 35160 . . . 35342) |
| E4_Orf6 | rc (34446 . . . 35327) | rc (34461 . . . 35342) |
| E4_Orf4 | rc (35230 . . . 35592) | rc (35245 . . . 35607) |
| E4_Orf3 | rc (35612 . . . 35962) | rc (35627 . . . 35977) |
| E4_Orf2 | rc (35962 . . . 36351) | rc (35977 . . . 36366) |
| E4_Orf1 | rc (36389 . . . 36769) | rc (36404 . . . 36784) |
| VA RNA I | (10496 . . . 10663) | (10496 . . . 10663) |
| VA RNA II | (10728 . . . 10901) | (10728 . . . 10901) |
| 5prime ITR | (1 . . . 73) | (1 . . . 73) |
| 3prime ITR | (37123 . . . 37195) | (37138 . . . 37210) |

TABLE 2b
Genomic boundaries of CDSs, RNAs and ITRs for GRAd21 and GRAd37. E3_CR1-alpha denotes
a putative open-reading frame having a GTG as initial codon. rc denotes reverse complement.
Products generated by splicing are indicated by multiple coordinate pairs.

| ORF | GRAd21 (SEQ ID NO: 10) | GRAd37 (SEQ ID NO: 14) |
|---|---|---|
| E1A | (551 . . . 1064, 1172 . . . 1461) | (551 . . . 1064, 1172 . . . 1461) |
| E1B_SmallT_19K | (1657 . . . 2208) | (1657 . . . 2208) |
| E1B_LargeT_55K | (1962 . . . 3473) | (1962 . . . 3473) |
| E1B_IX | (3567 . . . 3965) | (3567 . . . 3965) |
| E2A_DBP | rc (22470 . . . 24113) | rc (22470 . . . 24113) |
| E2B_IVa2 | rc (4176 . . . 5509, 5788 . . . 5800) | rc (4176 . . . 5509, 5788 . . . 5800) |
| E2B_Polymerase | rc (5130 . . . 8708, 13977 . . . 13985) | rc (5130 . . . 8708, 13977 . . . 13985) |
| E2B_pTP | rc (8510 . . . 10462, 13977 . . . 13985) | rc (8510 . . . 10462, 13977 . . . 13985) |
| L1_52-55KD | (10916 . . . 12139) | (10916 . . . 12139) |
| L1_IIIa | (12168 . . . 13952) | (12168 . . . 13952) |
| L2_Penton | (14022 . . . 15872) | (14022 . . . 15872) |
| L2_VII | (15905 . . . 16510) | (15905 . . . 16510) |
| L2_V | (16583 . . . 17659) | (16583 . . . 17659) |
| L2_X | (17691 . . . 17921) | (17691 . . . 17921) |
| L3_VI | (18022 . . . 18771) | (18022 . . . 18771) |
| L3_Hexon | (18878 . . . 21718) | (18878 . . . 21718) |
| L3_Endoprotease | (21743 . . . 22372) | (21743 . . . 22372) |
| L4_100kD | (24157 . . . 26664) | (24157 . . . 26664) |
| L4_22kD | (26348 . . . 26947) | (26348 . . . 26947) |
| L4_33kD | (26348 . . . 26693, 26974 . . . 27257) | (26348 . . . 26693, 26974 . . . 27257) |
| L4_VIII | (27318 . . . 27998) | (27318 . . . 27998) |
| E3_12.5K | (28002 . . . 28322) | (28002 . . . 28322) |
| E3_CR1-alpha | (28303 . . . 28848) | (28303 . . . 28848) |
| E3_gp19K | (29050 . . . 29532) | (29050 . . . 29532) |
| E3_CR1-beta | (29580 . . . 30446) | (29580 . . . 30446) |
| E3_CR1-gamma | (30492 . . . 30806) | (30492 . . . 30806) |
| E3_RID-alpha | (30818 . . . 31087) | (30818 . . . 31087) |
| E3_RID-beta | (31095 . . . 31512) | (31095 . . . 31512) |
| E3_14.7K | (31510 . . . 31893) | (31510 . . . 31893) |
| L5_Fiber | (32045 . . . 33838) | (32045 . . . 33838) |
| E4_Orf6-7 | rc (34027 . . . 34302, 35005 . . . 35187) | rc (34027 . . . 34302, 35005 . . . 35187) |
| E4_Orf6 | rc (34306 . . . 35187) | rc (34306 . . . 35187) |
| E4_Orf4 | rc (35090 . . . 35452) | rc (35090 . . . 35452) |
| E4_Orf3 | rc (35472 . . . 35822) | rc (35472 . . . 35822) |
| E4_Orf2 | rc (35822 . . . 36211) | rc (35822 . . . 36211) |
| E4_Orf1 | rc (36249 . . . 36629) | rc (36249 . . . 36629) |
| VA RNA I | (10493 . . . 10660) | (10493 . . . 10660) |
| VA RNA II | (10725 . . . 10898) | (10725 . . . 10898) |
| 5prime ITR | (1 . . . 78) | (1 . . . 78) |
| 3prime ITR | (36983 . . . 37060) | (36983 . . . 37060) |

TABLE 2c
Genomic boundaries of CDSs, RNAs and ITRs for GRAd33 and GRAd34. E3_CR1-alpha denotes
a putative open-reading frame having a GTG as initial codon. rc denotes reverse complement.
Products generated by splicing are indicated by multiple coordinate pairs.

| ORF | GRAd33 (SEQ ID NO: 16) | GRAd34 (SEQ ID NO: 20) |
|---|---|---|
| E1A | (546 . . . 1059, 1167 . . . 1456) | (546 . . . 1059, 1167 . . . 1456) |
| E1B_SmallT_19K | (1657 . . . 2211) | (1657 . . . 2211) |
| E1B_LargeT_55K | (1962 . . . 3473) | (1962 . . . 3473) |
| E1B_IX | (3567 . . . 3965) | (3567 . . . 3965) |
| E2A_DBP | rc (22636 . . . 24279) | rc (22636 . . . 24279) |
| E2B_IVa2 | rc (4027 . . . 5357, 5636 . . . 5648) | rc (4027 . . . 5357, 5636 . . . 5648) |
| E2B_Polymerase | rc (5130 . . . 8708, 13980 . . . 13988) | rc (5130 . . . 8708, 13980 . . . 13988) |
| E2B_pTP | rc (8510 . . . 10465, 13980 . . . 13988) | rc (8510 . . . 10465, 13980 . . . 13988) |
| L1_52-55KD | (10919 . . . 12142) | (10919 . . . 12142) |
| L1_IIIa | (12171 . . . 13955) | (12171 . . . 13955) |
| L2_Penton | (14025 . . . 15992) | (14025 . . . 15992) |
| L2_VII | (16024 . . . 16629) | (16024 . . . 16629) |
| L2_V | (16702 . . . 17784) | (16702 . . . 17784) |
| L2_X | (17816 . . . 18046) | (17816 . . . 18046) |
| L3_VI | (18147 . . . 18896) | (18147 . . . 18896) |
| L3_Hexon | (19003 . . . 21882) | (19003 . . . 21882) |
| L3_Endoprotease | (21907 . . . 22536) | (21907 . . . 22536) |
| L4_100kD | (24321 . . . 26816) | (24321 . . . 26816) |
| L4_22kD | (26500 . . . 27096) | (26500 . . . 27096) |
| L4_33kD | (26500 . . . 26845, 27123 . . . 27406) | (26500 . . . 26845, 27123 . . . 27406) |
| L4_VIII | (27468 . . . 28148) | (27467 . . . 28147) |
| E3_12.5K | (28151 . . . 28471) | (28151 . . . 28471) |
| E3_CR1-alpha | (28452 . . . 28997) | (28452 . . . 28997) |
| E3_gp19K | (29195 . . . 29674) | (29195 . . . 29674) |
| E3_CR1-beta | (29722 . . . 30588) | (29722 . . . 30588) |
| E3_CR1-gamma | (30634 . . . 30945) | (30634 . . . 30945) |
| E3_RID-alpha | (30957 . . . 31226) | (30957 . . . 31226) |
| E3_RID-beta | (31233 . . . 31652) | (31233 . . . 31652) |
| E3_14.7K | (31648 . . . 32031) | (31648 . . . 32031) |
| L5_Fiber | (32183 . . . 33988) | (32183 . . . 33988) |
| E4_Orf6-7 | rc (34179 . . . 34454, 35157 . . . 35339) | rc (34179 . . . 34454, 35157 . . . 35339) |
| E4_Orf6 | rc (34458 . . . 35340) | rc (34458 . . . 35339) |
| E4_Orf4 | rc (35242 . . . 35604) | rc (35242 . . . 35604) |
| E4_Orf3 | rc (35624 . . . 35974) | rc (35624 . . . 35974) |
| E4_Orf2 | rc (35974 . . . 36363) | rc (35974 . . . 36363) |
| E4_Orf1 | rc (36401 . . . 36781) | rc (36401 . . . 367801) |
| VA RNA I | (10496 . . . 10663) | (10496 . . . 10663) |
| VA RNA II | (10728 . . . 10901) | (10728 . . . 10901) |
| 5prime ITR | (1 . . . 73) | (1 . . . 73) |
| 3prime ITR | (37135 . . . 37207) | (37135 . . . 37207) |

TABLE 2d
Genomic boundaries of CDSs, RNAs and ITRs for GRAd35 and GRAd36. E3_CR1-alpha denotes
a putative open-reading frame having a GTG as initial codon. rc denotes reverse complement.
Products generated by splicing are indicated by multiple coordinate pairs.

| ORF | GRAd35 (SEQ ID NO: 18) | GRAd36 (SEQ ID NO: 5) |
|---|---|---|
| E1A | (546 . . . 1059, 1167 . . . 1456) | (546 . . . 1059, 1167 . . . 1456) |
| E1B_SmallT_19K | (1657 . . . 2211) | (1657 . . . 2211) |
| E1B_LargeT_55K | (1962 . . . 3473) | (1962 . . . 3473) |
| E1B_IX | (3567 . . . 3965) | (3567 . . . 3965) |
| E2A_DBP | rc (22636 . . . 24279) | rc (22636 . . . 24279) |
| E2B_IVa2 | rc (4027 . . . 5357, 5636 . . . 5648) | rc (4027 . . . 5357, 5636 . . . 5648) |
| E2B_Polymerase | rc (5130 . . . 8708, 13980 . . . 13988) | rc (5130 . . . 8708, 13980 . . . 13988) |
| E2B_pTP | rc (8510 . . . 10465, 13980 . . . 13988) | rc (8510 . . . 10465, 13980 . . . 13988) |
| L1_52-55KD | (10919 . . . 12142) | (10919 . . . 12142) |
| L1_IIIa | (12171 . . . 13955) | (12171 . . . 13955) |
| L2_Penton | (14025 . . . 15992) | (14025 . . . 15992) |
| L2_VII | (16024 . . . 16629) | (16024 . . . 16629) |
| L2_V | (16702 . . . 17784) | (16702 . . . 17784) |
| L2_X | (17816 . . . 18046) | (17816 . . . 18046) |
| L3_VI | (18147 . . . 18896) | (18147 . . . 18896) |
| L3_Hexon | (19003 . . . 21885) | (19003 . . . 21885) |
| L3_Endoprotease | (21910 . . . 22539) | (21910 . . . 22539) |
| L4_100kD | (24324 . . . 26819) | (24324 . . . 26819) |
| L4_22kD | (26503 . . . 27099) | (26503 . . . 27099) |
| L4_33kD | (26503 . . . 26848, 27126 . . . 27409) | (26503 . . . 26848, 27126 . . . 27409) |
| L4_VIII | (27470 . . . 28150) | (27470 . . . 28150) |
| E3_12.5K | (28154 . . . 28474) | (28154 . . . 28474) |
| E3_CR1-alpha | (28455 . . . 29000) | (28455 . . . 29000) |
| E3_gp19K | (29198 . . . 29677) | (29198 . . . 29677) |
| E3_CR1-beta | (29725 . . . 30591) | (29725 . . . 30591) |
| E3_CR1-gamma | (30637 . . . 30948) | (30637 . . . 30948) |
| E3_RID-alpha | (30960 . . . 31229) | (30960 . . . 31229) |
| E3_RID-beta | (31236 . . . 31655) | (31236 . . . 31655) |
| E3_14.7K | (31651 . . . 32034) | (31651 . . . 32034) |
| L5_Fiber | (32186 . . . 33991) | (32186 . . . 33991) |
| E4_Orf6-7 | rc (34182 . . . 34457, 35160 . . . 35342) | rc (34182 . . . 34457, 35160 . . . 35342) |
| E4_Orf6 | rc (34461 . . . 35342) | rc (34461 . . . 35342) |
| E4_Orf4 | rc (35245 . . . 35607) | rc (35245 . . . 35607) |
| E4_Orf3 | rc (35627 . . . 35977) | rc (35627 . . . 35977) |
| E4_Orf2 | rc (35977 . . . 36366) | rc (35977 . . . 36366) |
| E4_Orf1 | rc (36404 . . . 36784) | rc (36404 . . . 36784) |
| VA RNA I | (10496 . . . 10663) | (10496 . . . 10663) |
| VA RNA II | (10728 . . . 10901) | (10728 . . . 10901) |
| 5prime ITR | (1 . . . 73) | (1 . . . 73) |
| 3prime ITR | (37138 . . . 37210) | (37138 . . . 37210) |

TABLE 2e
Genomic boundaries of CDSs, RNAs and ITRs for GRAd37
and GRAd38. E3_CR1-alpha denotes a putative open-reading
frame having a GTG as initial codon. rc denotes reverse
complement. Products generated by splicing are indicated
by multiple coordinate pairs.

| ORF | GRAd38 (SEQ ID NO: 8) |
|---|---|
| E1A | (546 . . . 1059, 1167 . . . 1456) |
| E1B_SmallT_19K | (1657 . . . 2211) |
| E1B_LargeT_55K | (1962 . . . 3473) |
| E1B_IX | (3567 . . . 3965) |
| E2A_DBP | rc (22636 . . . 24279) |
| E2B_IVa2 | rc (4027 . . . 5357, 5636 . . . 5648) |
| E2B_Polymerase | rc (5130 . . . 8708, 13980 . . . 13988) |
| E2B_pTP | rc (8510 . . . 10465, 13980 . . . 13988) |
| L1_52-55KD | (10919 . . . 12142) |
| L1_IIIa | (12171 . . . 13955) |
| L2_Penton | (14025 . . . 15992) |
| L2_VII | (16024 . . . 16629) |
| L2_V | 16702 . . . 17784) |
| L2_X | 17816 . . . 18046) |
| L3_VI | (18147 . . . 18896) |
| L3_Hexon | (19003 . . . 21885) |
| L3_Endoprotease | (21910 . . . 22539) |
| L4_100kD | (24324 . . . 26819) |
| L4_22kD | (26503 . . . 27099) |
| L4_33kD | (26503 . . . 26848, 27126 . . . 27409) |
| L4_VIII | (27470 . . . 28150) |
| E3_12.5K | (28154 . . . 28474) |
| E3_CR1-alpha | (28455 . . . 29000) |
| E3_gp19K | (29198 . . . 29677) |
| E3_CR1-beta | (29725 . . . 30591) |
| E3_CR1-gamma | (30637 . . . 30948) |
| E3_RID-alpha | (30960 . . . 31229) |
| E3_RID-beta | (31236 . . . 31655) |
| E3_14.7K | (31651 . . . 32034) |
| L5_Fiber | (32186 . . . 33991) |
| E4_Orf6-7 | rc (34182 . . . 34457, 35160 . . . 35342) |
| E4_Orf6 | rc (34461 . . . 35342) |
| E4_Orf4 | rc (35245 . . . 35607) |
| E4_Orf3 | rc (35627 . . . 35977) |
| E4_Orf2 | rc (35977 . . . 36366) |
| E4_Orf1 | rc (36404 . . . 36784) |
| VA RNA I | (10496 . . . 10663) |
| VA RNA II | (10728 . . . 10901) |
| 5prime ITR | (1 . . . 73) |
| 3prime ITR | (37138 . . . 37210) |

### Aspects of the Invention and Particular Embodiments Thereof

The invention relates to several aspects as set out above in the summary of the invention. These aspects comprise alternative embodiments and preferred embodiments, which are described below.

In a first aspect, the invention provides a polynucleotide as described in the summary of the invention. Therein, the “variant thereof” refers to the recited amino acid fragments rather than to the entire recited SEQ ID NO. In a preferred embodiment, the HVR variant comprises one mutation. The polynucleotide is preferably an isolated polynucleotide. As known in the art, e.g. from Bradley et al. (J Virol., 2012 January; 86(2):1267-72), adenovirus neutralizing antibodies often target the hexon hypervariable regions, and by replacing the HVR regions of an adenovirus with serumprevalence, that adenovirus can evade the immune system in the immune host. Thus, while the above HVRs can be used with the respective hexon proteins defined below, they have utility independent from those hexon proteins and also from the below penton and fiber proteins, namely by replacing the hexon HVRs in a different adenovirus having other hexon, penton and/or fiber proteins.

Preferably, the hexon protein according to
A) comprises an amino acid sequence according to SEQ ID NO: 2, or a variant thereof,
B) comprises an amino acid sequence according to SEQ ID NO: 9, or a variant thereof,
C) comprises an amino acid sequence according to SEQ ID NO: 11, or a variant thereof,
D) comprises an amino acid sequence according to SEQ ID NO: 17, or a variant thereof,
E) comprises an amino acid sequence according to SEQ ID NO: 19, or a variant thereof
F) comprises an amino acid sequence according to SEQ ID NO: 21, or a variant thereof, and/or
G) comprises an amino acid sequence according to SEQ ID NO: 23, or a variant thereof.

In a preferred embodiment, the polynucleotide further encodes an adenoviral fiber protein and/or an adenoviral penton protein. Therein, the adenoviral fiber protein comprises with respect to
A) an amino acid sequence according to SEQ ID NO: 3 or SEQ ID NO: 6, or a variant thereof,
B), D), E) and/or F) an amino acid sequence according to SEQ ID NO: 6, or a variant thereof, and/or
C) an amino acid sequence according to SEQ ID NO: 12 or SEQ ID NO: 15, or a variant thereof. The adenoviral penton protein comprises with respect to
A) an amino acid sequence according to SEQ ID NO: 4 or SEQ ID NO: 7, or a variant thereof,
B), D), E), F) and/or G) an amino acid sequence according to SEQ ID NO: 7, or a variant thereof, and/or
C) an amino acid sequence according to SEQ ID NO: 13, or a variant thereof.

The hexon, fiber and penton variants of the above-described adenovirus hexon, fiber and penton proteins are capable of being integrated into an adenovirus capsid instead of the adenovirus hexon, fiber and penton proteins according to the respective SEQ ID NO and independently have a sequence identity (i.e. each variant can have a different sequence identity) of at least 80% sequence identity to the amino acid sequence defined by the respective SEQ ID NO, preferably at least 85%, 90%, 91%, 92%, 93%, 94%, 95%, 96%, 97%, 98%, 98.5%, 99%, 99.1%, 99.2%, 99.3%, 99.4%, 99.5%, 99.6%, 99.7%, 99.8% or at least 99.9%, wherein each higher value is preferred to any of the preceding lower values. Alternative to the definition by a percentage level of sequence identity, the hexon, fiber and penton variants can be defined to independently have a certain number of amino acid mutations within the respective SEQ ID NO (i.e. each variant can have a different number). The number of mutations is then as follows: up to 30, 25, 24, 23, 22, 21, 20, 19, 18, 17, 16, 15, 14, 13, 12, 10, 9, 8, 7, 6, 5, 4, 3, 2 or 1 mutation, wherein each lower value is preferred to any of the preceding higher values.

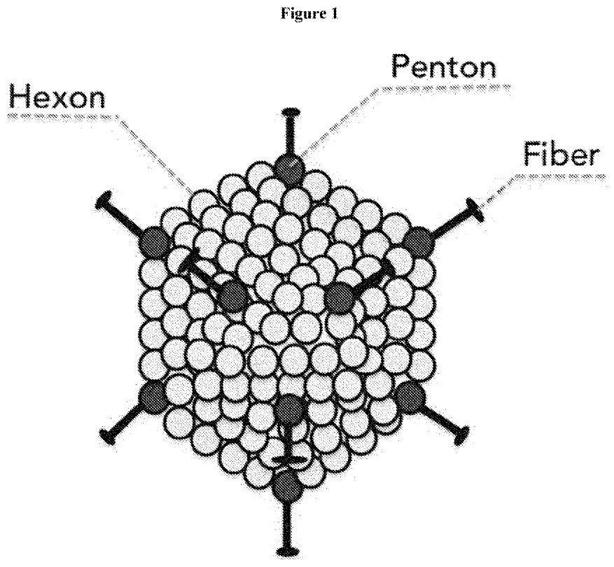

Each of the three capsid proteins hexon, fiber and penton (see also FIG. 1) have utility independent from the others, since they can replace the corresponding capsid protein in a different adenovirus having other hexon, penton and/or fiber proteins. Thus, in another aspect of the invention, the polynucleotide encodes one of the adenovirus hexon, fiber and penton proteins. In one embodiment, the polynucleotide encodes two of the adenovirus hexon, fiber and penton proteins, e.g. (i) the adenovirus hexon protein and the adenovirus fiber protein, (ii) the adenovirus hexon protein and the adenovirus penton protein, or (iii) the and/or the adenovirus fiber protein and the adenovirus penton protein. In a preferred embodiment, however, the polynucleotide encodes all of the adenovirus hexon, fiber and penton proteins.

It is preferred that the polynucleotide of the first aspect further comprises other adenoviral genes and nucleotide segments, which are adjacent to the hexon, penton and/or fiber gene in the adenovirus genome, using SEQ ID NOs 1, 5, 8, 10, 14, 16, 18, 20 and/or 22 as a reference. These are shown in Table 2. It is particularly preferred that the polynucleotide also comprises sequences required for packaging of the polynucleotide into an adenoviral particle.

Generally, it is preferred that the polynucleotide of the first aspect comprises at least one of the following:
(a) an adenoviral 5′-end, preferably an adenoviral 5′ inverted terminal repeat;
(b) an adenoviral Ela region, or a fragment thereof selected from the 13S, 12S and 9S regions;
(c) an adenoviral Elb region, or a fragment thereof selected from the group consisting of the E1b 19k, E1b 55k and IX regions;
(d) an adenoviral VA RNA region; or a fragment thereof selected from the group consisting of the VA RNA I and VA RNA II regions;
(e) an adenoviral E2b region; or a fragment thereof selected from the group consisting of the pTP, Polymerase and IVa2 regions;
(f) an adenoviral L1 region, or a fragment thereof, said fragment encoding an adenoviral protein selected from the group consisting of the 28.1 kD protein, polymerase, agnoprotein, 52/55 kDa protein, and Ma protein;
(g) an adenoviral L2 region, or a fragment thereof, said fragment encoding an adenoviral protein selected from the group consisting of the penton protein as defined above, VII, V, and X protein;
(h) an adenoviral L3 region, or a fragment thereof, said fragment encoding an adenoviral protein selected from the group consisting of the VI protein, hexon protein as defined above, and endoprotease;
(i) an adenoviral E2a region, or a fragment thereof, said fragment encoding an adenoviral protein consisting of the DBP protein;
(j) an adenoviral L4 region, or a fragment thereof said fragment encoding an adenoviral protein selected from the group consisting of the 100 kD protein, the 22 kD homolog, the 33 kD homolog, and VIII protein;
(k) an adenoviral E3 region, or a fragment thereof selected from the group consisting of E3 ORF1, E3 ORF2, E3 ORF3, E3 ORF4, E3 ORF5, E3 ORF6, E3 ORF7, E3 ORF8 and E3 ORF9;
(l) an adenoviral L5 region, or a fragment thereof said fragment encoding the fiber protein as defined above;
(m) an adenoviral E4 region, or a fragment thereof selected from the group consisting of E4 ORF6/7, E4 ORF6, E4 ORF5, E4 ORF4, E4 ORF3, E4 ORF2, and E4 ORF1; and/or
(n) an adenoviral 3′-end, preferably an adenoviral 3′ inverted terminal repeat.

These elements can be from the adenovirus according to SEQ ID NOs 1, 5, 8, 10, 14, 16, 18, 20 or 22 (i.e. as shown in Table 2), or from a different adenovirus, in particular from one of a different species, e.g. a human adenovirus, to form a chimeric adenovirus.

In some embodiments of the aforementioned polynucleotide it may be desirable that the polynucleotide does not comprise one or more genomic regions as outlined above (as in (a) to (m), such as e.g. region E3 and/or E4) and/or comprises an adenoviral gene which comprises a deletion and/or mutation which renders the at least one gene non-functional. In these preferred embodiments, the suitable adenoviral regions is modified to not include the aforementioned region(s)/gene(s) or to render the selected region(s)/gene(s) non-functional. One possibility to render them non-functional is to introduce one or more stop-codons (e.g. TAA) into the open reading frame of these genes. Methods of rendering the virus replication-defective are well known in the art (see e.g. Brody et al, 1994 Ann NY Acad Sci., 716: 90-101). A deletion can make space to insert transgenes, preferably within an expression cassette, such as a minigene cassette as described herein. Furthermore, deletions can be used to generate adenoviral vectors which are incapable to replicate without the use of a packaging cell line or a helper virus as is well known in the art. Thus, a final recombinant adenovirus comprising a polynucleotide as outlined above which comprises one or more of the specified gene/region deletions or loss-of-function mutations can provide a safer recombinant adenovirus for e.g. gene therapy or vaccination.

While the polynucleotide (i) may not comprise at least one genomic region/gene as outlined herein (such as e.g. region E3 and/or E4), specifically E1A, E1B, E2A, E2B, E3 ORF1, E3 ORF2, E3 ORF3, E3 ORF4, E3 ORF5, E3 ORF6, E3 ORF7, E3 ORF8, E3 ORF9, E4 ORF6/7, E4 ORF6, E4 ORF5, E4 ORF4, E4 ORF3, E4 ORF2 and/or E4 ORF1, preferably E1A, E1B, E2A, E2B, E3 and/or E4, and/or (ii) may comprise an adenoviral genomic region/gene (e.g. as specified for (i) above) which comprises a deletion and/or mutation which renders the at least one genomic region/gene non-functional, an intact E1A and/or E1B region may optionally be retained. Such an intact E1 region may be located in its native location in the adenoviral genome or placed in the site of a deletion in the native adenoviral genome (e.g., in the E3 region).

In a preferred embodiment, the polynucleotide of the first aspect further encodes one or more, preferably all of the following adenoviral proteins: protein VI, protein VIII, protein IX, protein Ma and/or protein IVa2.

An average person skilled in the art of adenoviruses is well aware of how to determine the open reading frames that encode for the above-specified adenoviral proteins. The skilled person is also aware of the structure of adenoviral genomes and can map, without undue burden, the individual adenoviral regions and ORFs outlined herein to any adenoviral genome.

In another embodiment, the polynucleotide of the first aspect further encodes one or more heterologous proteins or fragments thereof. The one or more heterologous proteins or fragments thereof are preferably non-adenoviral proteins or fragments thereof. In a preferred embodiment, the one or more non-adenoviral proteins or fragments thereof are one or more antigenic proteins or antigenic fragments thereof. Preferably, the one or more heterologous proteins or fragments thereof are encoded by a gene that is part of one or more expression cassettes. Sequences encoding for a heterologous protein and preferably an expression cassette comprising such sequence(s) encoding for a heterologous protein may be inserted into e.g. deleted regions of an adenoviral genome defined herein.

In a preferred embodiment, the heterologous protein or fragment thereof is a coronavirus protein or fragment thereof, more preferably a SARS-CoV-2 protein or fragment thereof. The term “SARS-CoV-2” preferably refers to any coronavirus strain that is classified as a strain of SARS-CoV-2 by the International Committee on Taxonomy of Viruses (ICTV). In addition or alternatively, it is a coronavirus with the sequence of the original strain of the 2019 outbreak “Severe acute respiratory syndrome coronavirus 2 isolate Wuhan-Hu-1” (NCBI Reference Sequence NC_045512.2, version of Mar. 30, 2020, based on Genbank Acc. No MN908947) or a variant thereof with at least 80%, 85%, 90%, 95%, 96%, 97%, 98% or preferably at least 99% sequence identity, wherein a higher value is preferred to any preceding lower values. Specifically, the protein or fragment thereof may be a coronavirus (preferably SARS-CoV-2) spike protein or fragment thereof, e.g. a spike protein (or fragment thereof) (i) comprising or consisting of a sequence according to SEQ ID NO: 30 or a variant thereof, and/or (ii) comprising or consisting of a polypeptide sequence encoded by a nucleotide sequence according to positions 6-3824 of SEQ ID NO: 29 or a variant thereof. The variant of SEQ ID NO: 29 or 30 has at least 80%, 85%, 90%, 91%, 92%, 93%, 94%, 95%, 96%, 97%, 98%, or 99% sequence identity to the respective SEQ ID NO, wherein a higher value is preferred to any preceding lower values. The variant is preferably functional, i.e. is capable of binding the human ACE2 protein.

In a preferred embodiment, the SARS-CoV-2 protein or variant thereof has one or more of the following amino acid mutations (including substitutions and deletions):
a) Asp 614 of SEQ ID NO: 30 (or the Asp of the corresponding position in the variant) substituted by Gly (SEQ ID NO: 24),
b) amino acids Lys 986 and Val 987 of SEQ ID NO: 30 (or the Lys and Val of the corresponding positions in the variant) both substituted by Pro (SEQ ID NO: 25),
c) of SEQ ID NO: 30, amino acids 69, 70 and 144 deleted, Asn 501 substituted by Tyr, Ala 570 substituted by Asp, Asp 614 substituted by Gly, Pro 681 substituted by His, Thr 716 substituted by Ile, Ser 982 substituted by Ala and Asp 1118 substituted by His,
d) of SEQ ID NO: 30, Leu 18 substituted by Phe, Asp 80 substituted by Ala, Asp 215 substituted by Gly, amino acids 242, 243 and 244 deleted, Lys 417 substituted by Asn, Glu 484 substituted by Lys, Asn 501 substituted by Tyr, Asp 614 substituted by Gly and Ala 701 substituted by Val,
e) of SEQ ID NO: 30, Leu18 substituted by Phe, Thr 20 substituted by Asn, Pro 26 substituted by Ser, Asp138 substituted by Tyr, Arg 190 substituted by Ser, Lys 417 substituted by Thr, Glu 484 substituted by Lys, Asn 501 substituted by Tyr, Asp 614 substituted by Gly, His 655 substituted by Tyr, Thr 1027 substituted by Ile and Val 1176 substituted by Phe,
f) of SEQ ID NO: 30, Ser 13 substituted by Ile, Trp 152 substituted by Cys, Leu 452 substituted by Arg and Asp 614 substituted by Gly,
g) of SEQ ID NO: 30, Gln 52 substituted by Arg, amino acids 69, 70 and 144 deleted, Glu 484 substituted by Lys, Gln 677 substituted by His and Phe 888 substituted by Leu,
h) of SEQ ID NO: 30, Leu 5 substituted by Phe, Thr 95 substituted by Ile, Asp 253 substituted by Gly, Asp 614 substituted by Gly, Ala 701 substituted by Val and Glu 484 substituted by Lys,
i) of SEQ ID NO: 30, Leu 5 substituted by Phe, Thr 95 substituted by Ile, Asp 253 substituted by Gly, Asp 614 substituted by Gly, Ala 701 substituted by Val and Ser 477 substituted by Asn,
j) of SEQ ID NO: 30, Thr 478 substituted by Lys, Asp 614 substituted by Gly, Pro 681 substituted by His and Thr 732 substituted by Ala,
k) of SEQ ID NO: 30, Thr 95 substituted by Ile, Gly 142 substituted by Asp, Glu 154 substituted by Lys, Leu 452 substituted by Arg, Glu 484 substituted by Gln, Asp 614 substituted by Gly, Pro 681 substituted by Arg and Gln 1071 substituted by His, and/or (preferably or)
l) of SEQ ID NO: 30, Thr 19 substituted by Arg, Gly 142 substituted by Asp, Glu 156 substituted by Gly, amino acids 157 and 158 deleted, Leu 452 substituted by Arg, Thr 478 substituted by Lys, Asp 614 substituted by Gly, Pro 681 substituted by Arg and Asp 950 substituted by Asn.

In other words, the SARS-CoV-2 protein may have the sequence as described in items a) to 1) above or a variant thereof at least 80%, 85%, 90%, 91%, 92%, 93%, 94%, 95%, 96%, 97%, 98%, or 99% sequence identity (maintaining the substitutions/deletions of items a) to 1)). For example the SARS-CoV-2 protein may have the sequence according to SEQ ID NO: 24 (Asp 614 to Gly substitution) or a variant thereof at least 80%, 85%, 90%, 91%, 92%, 93%, 94%, 95%, 96%, 97%, 98%, or 99% sequence identity (maintaining the substitution), optionally with the substitutions according to b), or it may have the sequence according to SEQ ID NO: (Lys 986 and Val987 to Pro substitutions) or a variant thereof at least 80%, 85%, 90%, 91%, 92%, 93%, 94%, 95%, 96%, 97%, 98%, or 99% sequence identity (maintaining the substitutions).

In one embodiment, the polynucleotide encodes an adenovirus, which preferably comprises an adenoviral genome comprising a polynucleotide of the first aspect. In a preferred embodiment, the adenoviral genome comprises the sequence according to SEQ ID NO 1, 5, 8, 14, 16, 18, 20 or 22, or a variant thereof having at least 80%, 85%, 90%, 91%, 92%, 93%, 94%, 95%, 96%, 97%, 98%, or 99% sequence identity, wherein a higher value is preferred to any preceding lower values. The term “encodes” in this respect does not require that the polynucleotide comprises only coding-sequences, as it may also comprise non-coding sequences, particularly of an adenovirus genome, preferably as described herein. Accordingly, the polynucleotide comprises coding and optionally also non-coding sequences of an adenovirus.

In a preferred embodiment, the encoded adenovirus is a replication-incompetent adenovirus, preferably comprising an adenoviral genome as specified above but that lacking one or more of the genomic regions/genes E1A, E1B, E2A, E2B, E3 and/or E4.

Most preferably, it encodes a recombinant adenovirus, preferably comprising an adenoviral genome according to SEQ ID NO 1, 5, 8, 10, 14, 16, 18, 20 or 22, ora variant thereof as defined above, preferably into which one or more genes encoding for the one or more heterologous proteins or fragments thereof are inserted (carrier adenovirus). Preferably, these one or more heterologous genes are inserted by replacing one or more of the genomic regions/genes E1A, E1B, E2A, E2B, E3 ORF1, E3 ORF2, E3 ORF3, E3 ORF4, E3 ORF5, E3 ORF6, E3 ORF7, E3 ORF8, E3 ORF9, E4 ORF6/7, E4 ORF6, E4 ORF5, E4 ORF4, E4 ORF3, E4 ORF2 and/or E4 ORF1, more preferably E1, E3 and/or E4. The heterologous genes are preferably inserted as part of an expression cassette. Optionally, the carrier adenovirus is also replication-incompetent as described herein, i.e. lacking one or more of the genomic regions/genes E1A, E1B, E2A, E2B, E3 and/or E4. For example, the recombinant adenovirus can be encoded by a sequence according to SEQ ID NO: 26, 27 or 28, or a variant thereof having at least 80% (preferably at least 80%, 95%, 96%, 97%, 98%, 99%, 99.5% or 99.9%) sequence identity, wherein a higher value is preferred to any preceding lower values, optionally into which the sequence of the one or more genes encoding for the one or more heterologous proteins or fragments thereof is inserted.

In an exemplary embodiment, the polynucleotide encodes an adenovirus, which comprises a polynucleotide according to SEQ ID NO: 31 (optionally with substitutions resulting in a spike protein according to SEQ ID NO: 25, e.g. Pos 2487 C->T, Pos 2488 A->G, Pos 2489 C->G, Pos 2490 C->A, Pos 2491 T->G, and Pos 2492 T->G), 32 or 33, or a variant thereof having at least 80%, preferably at least 80%, 95%, 96%, 97%, 98%, 99%, 99.5% or 99.9% sequence identity, wherein a higher value is preferred to any preceding lower values. Preferably therein, deletions as defined above for the adenovirus vector are taken into account, i.e. they do not contribute to reducing sequence identity.

In one embodiment, the polynucleotide encodes a recombinant adenovirus, wherein at least one adenoviral genomic region of the recombinant adenovirus is derived from an adenovirus not comprising one or more of the hexon, fiber and/or penton proteins as defined above (chimeric adenovirus). Preferably, the chimeric adenovirus is chimeric mainly or preferably only for one or more of the hexon, fiber and/or penton proteins. In other words, the polynucleotide encodes the one or more of the hexon, fiber and/or penton proteins as defined above, but one or more, preferably all other genomic regions are derived from a different adenovirus, in particular different from an adenovirus according to SEQ ID NO 1, 5, 8, 10, 14, 16, 18, 20 or 22. The different adenovirus is preferably one naturally found in a different host, more preferably a human adenovirus. This polynucleotide preferably encodes also for one or more heterologous non-adenoviral proteins or fragments thereof as defined above. Thus, one or more heterologous non-adenoviral genes are inserted into the adenoviral genome of the chimeric adenovirus. Accordingly, the adenoviral genome of the chimeric adenovirus is, except the DNA encoding one or more of the hexon, fiber and/or penton proteins as defined above, derived from a non-simian adenovirus, e.g. a human adenovirus, preferably a carrier non-simian, e.g. human, adenovirus.

It is generally preferred that the adenovirus is replication-incompetent. To this end, it is preferred that the adenovirus lacks one or more of the genomic regions E1 A, E1B, E2A, E2B, E3 and/or E4 or comprises a deletion and/or mutation therein which renders the genomic region or an expression product encoded by it non-functional.

In one particularly preferred embodiment, the polynucleotide, in all its variants described herein, may have a functionally impaired IVa2 gene, preferably a deletion of or a null-mutation in it. This gene is involved in viral DNA packing and its impairment leads to the production of virus-like particles. In this embodiment, the polynucleotide of the first aspect preferably encodes one or more non-adenoviral B-cell epitopes and/or T-cell epitopes.

In a second aspect, the invention provides a hexon polypeptide encoded by the polynucleotide as defined in A), B), C), D), E) or F) of the first aspect. Preferably, the hexon polypeptide is an isolated polypeptide.

In a third aspect, the invention provides an adenoviral capsid comprising the hexon protein encoded by the polynucleotide of the first aspect and preferably one or both of the fiber and penton proteins encoded by the polynucleotide of the first aspect. Preferably, the adenoviral capsid is an isolated adenoviral capsid.

Adenoviral capsid polypeptides and capsids can be obtained by expression in a cell. The expressed polypeptide(s) can be optionally purified using standard techniques. For example, the cells may be lysed either mechanically or by osmotic shock before being subject to precipitation and chromatography steps, the nature and sequence of which will depend on the particular recombinant material to be recovered. Alternatively, the expressed polypeptide(s) may be secreted and recovered from the culture medium in which the recombinant cells had been cultured as is known in the art of protein expression.

In a fourth aspect, the invention provides an adenovirus (also termed adenovirus vector or adenoviral vector herein) (i) encoded by a polynucleotide of the first aspect, (ii) comprising a polynucleotide according to the first aspect and/or (iii) comprising a hexon polypeptide of the second aspect or an adenoviral capsid of the third aspect. Preferably, the adenovirus is an isolated adenovirus.

Accordingly, the adenovirus can be, for example, an adenovirus encoded by SEQ ID NO 1, 5, 8, 10, 14, 16, 18, 20 or 22 or a recombinant adenovirus, such as a carrier or a chimeric adenovirus as defined above.

In an exemplary embodiment, the invention provides an adenovirus comprising a polynucleotide according to any one of SEQ ID NOs 26-28 and 31-33, or a variant thereof as defined above.

The adenovirus may or may not comprise a polynucleotide of the first aspect. In case this polynucleotide is not comprised in the adenovirus, it is preferred that it is provided in trans (i.e. by a genetic element that is not the adenovirus genome incorporated into the adenovirus). It is usually provided by a helper construct (e.g. a plasmid or virus) or by the genome of or a helper construct in a packaging host cell (complementing cell as described herein). It is further preferred that polynucleotides provided in trans are not comprised in the genome incorporated in the adenovirus, including homologs or other sequence variants of these polynucleotides. For example, if the polynucleotide provided in trans comprises a hexon, penton and/or fiber gene, the genome incorporated into the adenovirus does not comprise any polynucleotide encoding for a hexon, penton and/or fiber protein, respectively. Most preferably, the polynucleotide provided in trans encodes at least one (preferably all) adenoviral capsid polypeptide as defined herein.

In the construction of adenovirus vectors for delivery of a gene to a host, e.g. a human or other mammalian cell, a range of adenovirus nucleic acid sequences can be employed. For example, all or a portion of the adenovirus delayed early gene E3 may be eliminated from the adenovirus sequence which forms a part of the recombinant virus. The function of simian E3 is believed to be irrelevant to the function and production of the recombinant virus particle. In some embodiments, adenovirus vectors may also be constructed having a deletion of at least the ORF6 region of the E4 gene, and more desirably because of the redundancy in the function of this region, the entire E4 region. Still another vector of this invention may contain a deletion in the delayed early gene E2A. Deletions may also be made in any of the late genes L1 through L5 of the simian adenovirus genome. Similarly, deletions in the intermediate genes IX and IVa2 may be useful for some purposes. Other deletions may be made in the other structural or non-structural adenovirus genes. The above discussed deletions may be used individually, i.e., an adenovirus sequence for use in the present invention may contain deletions in only a single region. Alternatively, deletions of entire genes or portions thereof effective to destroy their biological activity may be used in any combination. For example, the adenovirus sequence may have deletions of the E1 and the E4 region, or of the E1, E2a and E3 region, or of the E1 and E3 regions, or of E1, E2A and E4 regions, with or without deletion of E3, and so on. Such deletions may be used in combination with other adenoviral gene mutations, such as temperature-sensitive mutations, to achieve a desired result.

An adenoviral vector lacking any essential adenoviral sequences (e.g., a region selected from E1 A, E1B, E2A, E2b, E4 ORF6, L1 or L4) may be cultured in the presence of the missing adenoviral gene products which are required for viral infectivity and propagation of an adenoviral particle. These helper functions may be provided by culturing the adenoviral vector in the presence of one or more helper constructs (e.g. a plasmid or virus) or a packaging host cell (complementing cell as described herein). See, for example, the techniques described for preparation of a “minimal” human adenovirus vector in WO96/13597).

Useful helper constructs contain selected adenovirus gene sequences that complement the respective genes that are deleted and/or that are not expressed by the vector and the cell in which the vector is transfected. In one embodiment, the helper construct is replication-defective and contains essential and optionally further adenovirus genes.

Helper constructs may also be formed into poly-cation conjugates as described in Wu et al, J. Biol. Chem., 264: 16985-16987 (1989); K. J. Fisher and J. M. Wilson, Biochem. J., 299: 49 (Apr. 1, 1994). A helper construct may optionally contain a reporter gene. A number of such reporter genes are known to the art. The presence of a reporter gene on the helper construct which is different from the transgene on the adenovirus vector allows both the adenovirus and the helper construct to be independently monitored. This second reporter may be used to facilitate separation between the resulting recombinant adenovirus and the helper construct upon purification. A preferred helper construct is a helper virus.

To generate recombinant adenoviruses (Ad) deleted in any of the genes described in the context of preferred embodiments herein, the function of the deleted gene region, if essential to the replication and infectivity of the virus, is preferably supplied to the recombinant virus by a helper construct or cell, i.e. a complementation or packaging cell. In many circumstances, a construct/cell expressing the human E1 can be used to transcomplement the vector used to generate recombinant adenoviruses. This is particularly advantageous because, due to the diversity between the polynucleotide sequences of the invention and the human adenoviral E1 sequences found in currently available packaging construct/cells, the use of the current human E1-containing constructs/cells will prevent the generation of replication-competent adenoviruses during the replication and production process. However, in certain circumstances, it will be desirable to utilize a construct/cell which expresses the E1 gene products for the production of an E1-deleted recombinant adenovirus.

If desired, one may utilize the sequences provided herein to generate a helper construct/cell or cell line that expresses, at a minimum, the adenovirus E1 gene from an adenovirus according to SEQ ID NO 1, 5, 8, 10, 14, 16, 18, 20 or 22 under the transcriptional control of a promoter for expression in a selected parent cell line, such as e.g. a HeLa cell. Inducible or constitutive promoters may be employed for this purpose. Examples of promoters are provided e.g. in the examples described herein. Such E1-expressing cells are useful in the generation of recombinant adenovirus E1 deleted vectors. Additionally, or alternatively, the invention provides constructs/cells that express one or more adenoviral gene products, e.g., E1 A, E1B, E2A, and/or E4 ORF6, preferably Ad5 E4 ORF6, which can be constructed using essentially the same procedures for use in the generation of recombinant adenoviral vectors. Such constructs/cells can be utilized to transcomplement adenovirus vectors deleted in essential genes that encode those products, or to provide helper functions necessary for packaging of a helper-dependent virus (e. g., adeno-associated virus).

Generally, when delivering an adenovirus vector by transfection, the vector is delivered in an amount from about 0.1 μg to about 100 μg DNA, and preferably about 10 to about 50 μg DNA to about 1×10 4 cells to about 1×10 3 cells, and preferably about 10\hat{ }5 cells. However, the relative amounts of vector DNA to host cells may be adjusted, taking into consideration such factors as the selected vector, the delivery method and the host cells selected. Introduction of the vector into a host cell may be achieved by any means known in the art or as disclosed herein, including transfection, and infection, e.g. using CaPO₄ transfection or electroporation.

For the construction and assembly of the desired recombinant adenovirus, the adenovirus vector can in one example be transfected in vitro in the presence of a helper construct into the packaging cell line, allowing homologous recombination to occur between the helper and the adenovirus vector sequences, which permits the adenovirus-transgene sequences in the vector to be replicated and packaged into virion capsids, resulting in the recombinant viral vector particles as is well known in the art. A recombinant adenovirus of the invention is useful e.g. in transferring a selected transgene into a selected host cell.

In a preferred embodiment, the adenovirus of the fourth aspect has a seroprevalence in less than 5% of human subjects and preferably no seroprevalence in human subjects, most preferably no seroprevalence in human subjects that have not previously been in contact with a non human great apes adenovirus, more preferably with one or more adenoviruses according to SEQ ID NO: 1, 5, 8, 10, 14, 16, 18, 20 and/or 22. In this context it is preferred that the human subjects belong to an ethnic group selected from the group consisting of Europeans, indigenous people of Africa, Asians, indigenous people of America and indigenous people of Oceania. Methods for the identification of the ethnic origin of a human subject are comprised in the art (see e.g. WO 2003/102236).

In a further preferred embodiment of a recombinant adenovirus, the adenovirus is capable of entering a mammalian target cell, i.e. it is infectious. An infectious recombinant adenoviruses of the invention can be used as a vaccine and for gene therapy as also described herein. Thus, in another embodiment it is preferred that the recombinant adenovirus comprises a molecule for delivery into a target cell. Preferably, the target cell is a mammalian cell, e.g. a non human great apes cell, a rodent cell or a human cell. For example, the molecule for delivery into a target cell can be a polynucleotide encoding for a heterologous protein (i.e. a heterologous gene) as defined herein, preferably within an expression cassette. Methods to introduce an expression cassette into the genome of an adenovirus are well known in the art. In one example a recombinant adenovirus of the present invention that comprises an expression cassette, encoding e.g. a heterologous gene, can be generated by replacing a genomic region of the adenovirus selected from E1A, E1B, E2A, E2B, E3 and/or E4 with said expression cassette. The genomic regions E1A, E1B, E2A, E2B, E3 and E4 of the adenoviruses of the invention can easily be identified by an alignment with known and annotated adenoviral genomes such as from human Ad5 (see: Birgitt Täuber and Thomas Dobner, Oncogene (2001) 20, p. 7847-7854; and also: Andrew J. Davison, et al., Journal of General Virology (2003), 84, p. 2895-2908).

The molecule for delivery into a target cell is preferably a heterologous polynucleotide but may also be a polypeptide or a small chemical compound, preferably having a therapeutic or diagnostic activity. In one particularly preferred embodiment, the molecule for delivery into a target cell is a heterologous polynucleotide that comprises an adenovirus 5′ inverted terminal repeat sequence (ITR) and a 3′ ITR. It will be evident to the skilled person that the molecular size of the molecule has to be chosen such that the capsid can form around and package the molecule, when the recombinant adenovirus is produced, e.g. in a packaging cell. Thus, preferably the heterologous gene is a minigene which can have e.g. up to 7000 or up to 8000 base pairs.

In a fifth aspect, the invention provides a virus-like particle (VLP) (i) encoded by a polynucleotide of the first aspect and/or (ii) comprising a hexon polypeptide of the second aspect or the capsid of the third aspect. Preferably, the VLP is an isolated VLP.

In one embodiment, the polynucleotide encoding the VLP has the IVa2 gene deleted or has a null-mutation in the IVa2 gene.

According to the definition of VLPs below, the VLP of the fifth aspect comprises substantially no adenoviral genomic DNA. VLPs, including adenovirus VLPs, have been used for vaccination, gene therapy or for direct drug delivery, e.g. of anti-cancer drugs (Chroboczek et al., ACTA ABP BIOCHIMICA POLONICA, Vol. 61, No. 3/2014). Accordingly, the VLP of the fourth aspect may comprises one or more heterologous genes as defined above, or one or more B-cell and/or T-cell epitopes thereof. In another embodiment, it may comprise one or more non-adenoviral genes for gene therapy, and/or one or more pharmaceutical agents, e.g. anti-cancer agents. In one embodiment, the VLP incorporates, preferably presents one or more heterologous proteins or fragments (preferably B-cell and/or T-cell epitopes) thereof as defined above.

In a sixth aspect, the invention provides a vector comprising a polynucleotide of the first aspect. Preferably, the vector is an isolated vector. In a preferred embodiment, the vector is a plasmid vector, e.g. an expression vector. A plasmid vector can advantageously be used to generate a recombinant adenovirus as described herein. As the sequence information of the novel hexon, penton and fiber proteins of the invention are provided, said recombinant adenovirus is obtainable e.g. by constructing a recombinant adenovirus which is encoded by the polynucleotide of the first aspect and any other adenoviral genomic region. Methods for the construction of recombinant adenoviruses are well known in the art. Useful techniques for the preparation of recombinant adenoviruses are, for example, reviewed in Graham & Prevec, 1991 In Methods in Molecular Biology: Gene Transfer and Expression Protocols, (Ed. Murray, EJ.), p. 109; and Hitt et al., 1997, Advances in Pharmacology 40:137-206. Further methods are described in WO 2006/086284.

In order to express a polynucleotide of the first aspect, one can subclone said polynucleotide into an expression vector that contains a strong promoter to direct transcription, preferably with an expression cassette. Suitable bacterial promoters are well known in the art, e.g., E. coli, Bacillus sp., and Salmonella, and kits for such expression systems are commercially available. Similarly eukaryotic expression systems for mammalian cells, yeast, and insect cells are well known in the art and are also commercially available. See below for further details of expression cassettes.

The particular expression vector useful for transporting the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ, but there are many more known in the art to the skilled person that can be usefully employed. Expression vectors containing regulatory elements from eukaryotic viruses are typically used in eukaryotic expression vectors, e.g. SV40 vectors, papilloma virus vectors, and vectors derived from Epstein-Barr virus. Other exemplary eukaryotic vectors include pMSG, pAV009/A. sup. +, pMT010/A. sup.+, pMAMneo-5, baculovirus pDSVE, pcDNA3.1, pIRES and any other vector allowing expression of proteins under the direction of e.g. the HCMV immediate-early promoter, SV40 early promoter, SV40 late promoter, metallothionein promoter, murine mammary tumor virus promoter, Rous sarcoma virus promoter, polyhedrin promoter, or other promoters shown effective for expression in eukaryotic cells. Some expression systems have markers that provide gene amplification such as thymidine kinase, hygromycin B phosphotransferase, and dihydrofolate reductase. Alternatively, high yield expression systems not involving gene amplification are also suitable. The elements that may also be included in expression vectors include a replicon that functions in E. coli, a gene encoding drug resistance to permit selection of bacteria that harbour recombinant plasmids, and unique restriction sites in nonessential regions of the plasmid to allow insertion of eukaryotic sequences. The particular drug resistance gene chosen is not critical—any of the many drug resistance genes known in the art are suitable. The prokaryotic sequences are optionally chosen such that they do not interfere with the replication of the DNA in eukaryotic cells, if necessary.

In a seventh aspect, the invention provides a composition comprising (i) an adjuvant, (ii) a polynucleotide of the first aspect, a hexon polypeptide of the second aspect, an adenoviral capsid of the third aspect, an adenovirus of the fourth aspect, a virus-like particle of the fifth aspect, or a vector of the sixth aspect, and optionally (iii) a pharmaceutically acceptable excipient.

Preferably, the adjuvant is an agonist for a receptor selected from the group consisting of type I cytokine receptors, type II cytokine receptors, TNF receptors, vitamin D receptor acting as transcription factor, and the Toll-like receptors 1 (TLR1), TLR-2, TLR 3, TLR4, TLR5, TLR-6, TLR7 and TLR9.

A composition that comprises an adjuvant can be used as a vaccine, e.g. for human subjects. For instance, activation of specific receptors can stimulate an immune response. Such receptors are known to the skilled artisan and comprise, for example, cytokine receptors, in particular type I cytokine receptors, type II cytokine receptors, TNF receptors; and vitamin D receptor acting as transcription factor; and the Toll-like receptors 1 (TLR1), TLR-2, TLR 3, TLR4, TLR5, TLR-6, TLR7, and TLR9. Agonists to such receptors have adjuvant activity, i.e., are immunostimulatory. In a preferred embodiment, the adjuvant of the composition may be one or more Toll-like receptor agonists. In a more preferred embodiment, the adjuvant is a Toll-like receptor 4 agonist. In a particular preferred embodiment, the adjuvant is a Toll-like receptor 9 agonist. For adjuvant examples, see below. Also, preferred pharmaceutically acceptable excipients are mentioned below.

In an eighth aspect, the invention provides a cell comprising a polynucleotide of the first aspect, a hexon polypeptide of the second aspect, an adenoviral capsid polypeptide of the third aspect, an adenovirus of the fourth aspect, a virus-like particle of the fifth aspect, or a vector of the sixth aspect. Preferably, the cell is an isolated cell.

Preferably, the cell is a host cell that expresses at least one adenoviral gene, or preferably all adenoviral genes, that is/are deleted or rendered non-functional as explained above to render the adenovirus replication-incompetent. By expression of this at least one genes, the host cell preferably enables replication of the otherwise replication-incompetent adenovirus. In one embodiment, the host cell that expresses at least one adenoviral gene selected from the group consisting of E1A, E1B, E2A, E2B, E3 and E4. In particular, this at least one adenoviral gene is deleted or rendered non-functional in the adenoviral genome. Such a complement cell can be used for the propagation and rescue of adenoviruses that are replication-incompetent, because they lack e.g. one of the aforementioned gene products.

The cell may be selected of a bacterial cell such as an E. coli cell, a yeast cell such as Saccharomyces cerevisiae or Pichia pastoris, a plant cell, an insect cell such as SF9 or Hi5 cells, or a mammalian cell. Preferred examples of mammalian cells are Chinese hamster ovary (CHO) cells, human embryonic kidney (HEK 293) cells, HELA cells, human hepatoma cells (e.g. Huh7.5), Hep G2 human hepatoma cells, Hep 3B human hepatoma cells and the like.

If the cell comprises a polynucleotide according to the first aspect, this polynucleotide may be present in the cell either (i) freely dispersed as such, or (ii) integrated into the cell genome or mitochondrial DNA.

In a further preferred embodiment, the cell is a host cell, preferably a HEK 293 cell or a PER. C6™ cell, that expresses at least one adenoviral gene selected from the group consisting of E1 A, E1B, E2A, E2B, E4, L1, L2, L3, L4 and L5.

Standard transfection methods can be used to produce bacterial, mammalian, yeast or insect cell lines. Any of the well-known procedures for introducing foreign polynucleotide sequences into host cells may be used. For example, commercially available liposome-based transfection kits such as Lipofectamine™ (Invitrogen), commercially available lipid-based transfection kits such as Fugene (Roche Diagnostics), polyethylene glycol-based transfection, calcium phosphate precipitation, gene gun (biolistic), electroporation, or viral infection and any of the other well-known methods for introducing cloned genomic DNA, cDNA, synthetic DNA or other foreign genetic material into a host cell may be used. It is only necessary that the particular genetic engineering procedure used be capable of successfully introducing at least one gene into the host cell capable of expressing the receptor.

Further embodiments of the cell are described with respect to the third aspect of the invention above.

In a ninth aspect, the invention provides a polynucleotide of the first aspect, a hexon polypeptide of the second aspect, an adenoviral capsid polypeptide of the third aspect, an adenovirus of the fourth aspect, a virus-like particle of the fifth aspect, a vector of the sixth aspect, a composition of the seventh aspect and/or a cell of the eighth aspect for use in treating or preventing a disease.

In one embodiment, the treating or preventing is by vaccination. In another embodiment, the treating is by gene therapy. With respect to vaccination, the disease is an infectious disease, preferably caused by a pathogen as described herein, or a non-infectious disease, preferably characterized by diseased cells that express antigens not expressed by healthy cells (such as tumor cells expressing tumor-associated antigens). With respect to gene therapy, the disease is an inheritable disease caused by one or more somatic mutations leading to a loss or gain of function of a gene or protein. In a preferred embodiment, the use is for treating or preventing a coronavirus disease. The terms “coronavirus disease” is distinguished herein from coronavirus infection (entry of coronavirus into at least one cell of a subject and its replication in the at least one cell) by the presence of at least one coronavirus disease symptom. As long as the infection is not accompanied by at least one symptom of coronavirus disease, it (or the subject) is asymptomatic (includes presymptomatic). The term coronavirus disease as used herein requires the presence of a coronavirus infection and at least one symptom of coronavirus disease (also referred to herein as symptomatic infection). Coronavirus symptoms include dry cough, fever (≥37.8° C.), runny and/or blocked nose, fatigue, breathing difficulty, pneumonia, organ (e.g. heart, lung, liver and/or kidney) failure, itchy throat, headache, joint pain, nausea, diarrhoea, shivering, lymphophenia, loss of smell and/or loss of taste. Preferably, the coronavirus disease is characterized by the presence of two or more, three or more, or four or more symptoms, preferably including one or two or more of dry cough, fever (≥37.8° C.), breathing difficulty, loss of smell and/or loss of taste. The coronavirus disease is preferably a respiratory disease (e.g. SARS or MERS), more preferably SARS, most preferably Covid-19.

It is well-known that adenoviruses are useful in gene-therapy and as vaccines. Preclinical and clinical studies have demonstrated the feasibility of vector design, robust antigen expression and protective immunity using this system. Thus, a preferred embodiment of the use is in vaccination, e.g. for human subjects. Detailed instructions of how adenoviruses are used and prepared for vaccination are provided as ample literature comprised in the art and known to the skilled person. Viral vectors based e.g. on a non human great apes adenovirus represent an alternative to the use of human derived Ad vectors for the development of genetic vaccines (Farina S F, J Virol. 2001 December; 75(23):11603-13; Fattori E, Gene Ther. 2006 July; 13(14):1088-96). Adenoviruses isolated from non human great apes are closely related to adenoviruses isolated from humans as demonstrated by their efficient propagation in cells of human origin. However, since human and non human apes adenoviruses are related, there may be some degree of or no serologic cross reactivity between the two virus species. This presumption has been confirmed when chimpanzee adenoviruses were isolated and characterized. Thus, a non human great apes adenovirus according to the invention provides a basis for reducing the adverse effects associated with the preexisting immunity in humans to common serotypes of human adenoviruses, and thereby a valuable medical tool that can e.g. be used for immunization and/or gene therapy.

This is due to the novel sequences of adenovirus capsid proteins including hexon, penton and fiber protein. Accordingly, no or very few neutralizing antibodies specific for the capsid proteins according to the invention are expected to be present in human blood sera. Thus, one advantage of the novel sequences is that they can be used to enhance prior art adenoviruses, which have been engineered for e.g. medical purposes. For example, the sequences can be used to e.g. replace/substitute one or more of the major structural capsid proteins of a different adenovirus, e.g. a prior art adenovirus, to obtain improved recombinant adenoviruses with a reduced seroprevalence in humans (chimeric adenoviruses). As the novel sequences and therefore adenoviruses which have been re-engineered as described will not encounter any significant inhibitory immune response in humans when administered, their overall transduction efficiency and infectivity will be enhanced. Thus, such improved adenoviruses are expected to be more effective vaccines as the entry into host cells and the expression of antigens will not be hampered by any significant titer of neutralizing antibodies.

It is preferred that the vaccine comprises an adjuvant. Preferred immunological adjuvants are mentioned herein and can be used in such a vaccine.

If the use is a vaccination, a recombinant adenovirus of the invention can be administered in an immunologically and/or prophylactically effective dose which is preferably 1×10⁸ to 1×10¹¹ viral particles (i.e., 1×10⁸, 5×10⁸, 1×10⁹, 5×10⁹, 1×10¹⁰, 2.5×10¹⁰ or 5×10¹⁰ particles).

Furthermore, for a vaccination which requires a boosting, it is preferred to apply a “heterologous prime-boost” methodology: In vaccination, the agents of any one of the first to ninth aspect (polynucleotide, hexon polypeptide, adenoviral capsid polypeptide, adenovirus, VLP, vector, composition, cell, respectively) may be used for priming or for boosting, in particular for a heterologous prime-boost vaccination. In a preferred embodiment of heterologous prime-boost two different vaccines, e.g. adenoviruses may be used, wherein it is particularly advantageous that the agent of any one of the first to ninth aspect is used as the boost vaccine due to the lack or neutralizing antibodies in e.g. humans.

A recombinant adenovirus prepared using a polynucleotide or recombinant adenoviral protein or fragment thereof according to the invention can be used to transduce a host cell with a polynucleotide, e.g. DNA. Thus, a preferably replication deficient, albeit infectious (i.e. capable of entering a host cell) adenovirus can be prepared to express any custom protein or polypeptide in a host cell. Thus, in a preferred embodiment, the therapy recited in the use according to the invention is gene therapy. The gene therapy may be an in vivo, ex vivo, or in vitro gene therapy. Preferably, it is a somatic gene therapy. If an agent of any one of the first to ninth aspect is used for gene therapy and is administered to a subject to be treated, it is preferred that it is administered in a sufficiently large dose such that the treatment results in one or more cells of the patient being transfected, i.e. transduced. If a recombinant adenovirus, VLP and/or a pharmaceutical composition according to the invention is administered by any of the preferred means of administrations disclosed herein, it is preferred that an effective dose which is preferably 1×10⁸ to 5×10¹¹ viral particles (i.e., 1×10⁸, 5×10⁸, 1×10⁹, 5×10⁹, 1×10¹⁰, 2.5×10¹⁰, 5×10¹⁰, 1×10¹¹ or, most preferably, 5×10¹¹ particles) is administered. In preferred embodiments, the preferably heterologous polynucleotide that is comprised in the recombinant adenovirus of the invention is capable of expressing a protein or polypeptide in a host cell of the subject, wherein the protein or polypeptide comprises a signal peptide which effects secretion of the protein or polypeptide from said host cell. For example, a patient in need of a certain protein can be treated using an adenovirus of the present invention which comprises a cDNA that encodes a secretable form of that protein.

In a further embodiment of the use of the present invention, an agent of any one of the first to ninth aspect (in the following also referred to as pharmaceutical according to the invention) is formulated to further comprise one or more pharmaceutically acceptable diluents; carriers; excipients, including fillers, binders, lubricants, glidants, disintegrants, and adsorbents; and/or preservatives.

The pharmaceutical according to the invention can be administered by various well known routes, including oral, rectal, intragastrical and parenteral administration, e.g. intravenous, intramuscular, intranasal, intradermal, subcutaneous and similar administration routes. Parenteral-, intramuscular- and intravenous administration is preferred. Preferably the pharmaceutical according to the invention is formulated as syrup, an infusion or injection solution, a tablet, a capsule, a capslet, lozenge, a liposome, a suppository, a plaster, a band-aid, a retard capsule, a powder, or a slow release formulation. Preferably the diluent is water, a buffer, a buffered salt solution or a salt solution and the carrier preferably is selected from the group consisting of cocoa butter and vitebesole.

Particular preferred pharmaceutical forms for the administration of the pharmaceutical according to the invention during the use of the present invention are forms suitable for injectable use and include sterile aqueous solutions or dispersions and sterile powders for the extemporaneous preparation of sterile injectable solutions or dispersion. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils.

Infusion or injection solutions can be accomplished by any number of art-recognized techniques including but not limited to addition of preservatives like anti-bacterial or anti-fungal agents, e.g. parabene, chlorobutanol, phenol, sorbic acid or thimersal. Further, isotonic agents, such as sugars or salts, in particular sodium chloride may be incorporated in infusion or injection solutions.

Preferred diluents of the present invention are water, physiological acceptable buffers, physiological acceptable buffer salt solutions or salt solutions. Preferred carriers are cocoa butter and vitebesole. Excipients which can be used with the various pharmaceutical forms of the pharmaceutical according to the invention can be chosen from the following non-limiting list:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glyceride and sodium stearyl fumarates,
c) disintegrants such as starches, croscaramellose, sodium methyl cellulose, agar, bentonite, alginic acid, carboxymethyl cellulose, polyvinyl pyrrolidone and the like.

Other suitable excipients can be found in the Handbook of Pharmaceutical Excipients, published by the American Pharmaceutical Association.

Certain amounts of the pharmaceutical according to the invention are preferred for the therapy or prophylaxis of a disease. It is, however, understood that depending on the severity of the disease, the type of the disease, as well as on the respective patient to be treated, e.g. the general health status of the patient, etc., different doses of the pharmaceutical according to the invention are required to elicit a therapeutic or prophylactic effect. The determination of the appropriate dose lies within the discretion of the attending physician. If the pharmaceutical according to the invention is to be used prophylactically, it may be formulated as a vaccine. In this case the pharmaceutical according to the invention is preferably administered in above outlined preferred and particular preferred doses. Preferably, the administration of the vaccine is repeated at least two, three, four, five, six, seven, eight nine or at least 10 times over the course of a defined period of time, until the vaccinated subject has generated sufficient antibodies against the pharmaceutical according to the invention so that the risk of developing the respective disease has lessened. The period of time in this case is usually variable depending on the antigenicity of the vaccine. Preferably the period of time is not more than four weeks, three months, six months or three years. In one embodiment, if an adenovirus according to the invention is used for vaccination purposes, at least one of the hypervariable domains of the hexon protein can be replaced by an immunogenic epitope of the respective disease agent that the vaccination is directed against. Vaccines typically contain one or more adjuvants as outlined above. A detailed summary of the use of adenoviruses for vaccination and methods pertaining thereto is provided in: Bangari D S and Mittal S K (2006) Vaccine, 24(7), p. 849-862; see also: Zhou D, et al., Expert Opin Biol Ther. 2006 January; 6(1):63-72; and: Folgori A, et al., Nat Med. 2006 February; 12(2):190-7; see also: Draper S J, et al., Nat Med. 2008 August; 14(8):819-21. Epub 2008 Jul. 27.

In a tenth aspect, the present invention relates to an in vitro method for producing an adenovirus or an adenovirus-like particle, comprising the steps of
(i) expressing a polynucleotide of the first aspect in a cell such that an adenovirus or an adenovirus-like particle is assembled in the cell,
(ii) isolating the adenovirus or the adenovirus-like particle from the cell or the medium surrounding the cell.

The method optionally comprises a further step prior to step (i) of introducing the polynucleotide of the first aspect or a vector of the sixth aspect into the cell, e.g. as described above.

It is generally preferred that the polynucleotide encodes an adenovirus of the fourth aspect or a virus-like particle of the fifth aspect. The adenovirus is preferably replication-incompetent. The cell is preferably a cell of the eighth aspect. If the polynucleotide encodes a replication-incompetent adenovirus, it is preferred that the cell is a helper cell or comprises a helper construct (e.g. a helper plasmid or helper virus, e.g. as it is transduced with a helper construct, preferably infected with a helper virus, prior to or during step (i)) as described herein, wherein the helper cell or the helper construct, respectively, expresses the genes/genomic regions that render the adenovirus replication-incompetent.

“Such that an adenovirus or an adenovirus-like particle is assembled in the cell” means that in step (i), all genes necessary for assembling the adenovirus or the adenovirus-like particle, as described herein, are expressed in the cell. This comprises all genes necessary for packaging the adenovirus (i.e. packaging the genome into the virus capsid) if an adenovirus is to be assembled.

In a further aspect, the present invention relates to
(i) an isolated polynucleotide encoding for an adenovirus,
(ii) an isolated adenovirus,
(iii) a virus-like particle (VLP) comprising an adenovirus capsid,
(iv) an isolated vector comprising (i),
(v) an isolated cell comprising any one of (i) to (iv),
(vi) a composition comprising an adjuvant, any one of (i) to (v), and optionally (iii) a pharmaceutically acceptable excipient,
(vii) any one of (i) to (vi) for use in treating or preventing coronavirus disease, and (viii) an in vitro method for producing an adenovirus or an adenovirus-like particle, comprising the steps of
   (a) expressing a polynucleotide encoding for an adenovirus in a cell such that an adenovirus or an adenovirus-like particle is assembled in the cell,
   (b) isolating the adenovirus or the adenovirus-like particle from the cell or the medium surrounding the cell,
      all wherein the adenovirus, the polynucleotide encoding for it or the VLP comprise a coronavirus spike gene or protein as defined above. Preferably, the coronavirus spike gene is comprised in the adenovirus genome.

The adenovirus vector can be derived from any adenovirus, including but not limited to those mentioned herein, for example Ad5, Ad11, Ad26, Ad35, Ad49, ChAd3, ChAd4, ChAd5, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63 and ChAd82, which are preferably replication-incompetent, or Ad4 and Ad7, which may be replication-competent.

All embodiments and definitions given herein above and below, in as far as they are applicable to any adenovirus comprising a coronavirus spike gene or protein, also apply to this further aspect of the invention.

### Definitions and Further Embodiments of the Invention

In the following, some definitions of terms frequently used in this specification are provided. These terms will, in each instance of its use, in the remainder of the specification have the respectively defined meaning and preferred meanings.

As used herein, the term “isolated” refers to a molecule which is substantially free of other molecules with which it is naturally associated with. In particular, isolated means the molecule is not in an animal body or an animal body sample. An isolated molecule is thus free of other molecules that it would encounter or contact in an animal. Isolated does not mean isolated from other components associated with as described herein, e.g. not isolated from other components of a composition the molecule is comprised in, or isolated from a vector or cell it is comprised in.

The term “polynucleotide” is intended to refer to a nucleic acid, i.e. a biological molecule made up of a plurality of nucleotides. It includes DNA, RNA and synthetic analogs, e.g. PNA. DNA is preferred.

The term “open reading frame” (ORF) refers to a sequence of nucleotides that can be translated into amino acids. Typically, an ORF contains a start codon, a subsequent region usually having a length which is a multiple of 3 nucleotides, but does not contain a stop codon (TAG, TAA, TGA, UAG, UAA, or UGA) in the given reading frame. An ORF codes for a protein where the amino acids into which it can be translated form a peptide-linked chain.

As used herein, the term “protein”, “peptide”, “polypeptide”, “peptides” and “polypeptides” are used interchangeably throughout. These terms refers to both naturally occurring peptides, e.g. naturally occurring proteins and synthesized peptides that may include naturally or non-naturally occurring amino acids. Peptides can be also chemically modified by modifying a side chain or a free amino or carboxy-terminus of a natural or non-naturally occurring amino acid. This chemical modification includes the addition of further chemical moieties as well as the modification of functional groups in side chains of the amino acids, such as a glycosylation. A peptide is a polymer preferably having at least 3, 4, 5, 6, 7, 8, 9, 10, 15, 25, 30, 35, 40, 45, 50, 55, 60, 65, 70, 75, 80, 85, 90, 95, or at least 100 amino acids, most preferably at least 8 or at least 30 amino acids. As the polypeptides and proteins disclosed herein are derived from adenovirus, it is preferred that the molecular mass of an isolated polypeptide or protein as used herein does not exceed 200 kDa.

An adenovirus (Ad) is a non-enveloped, icosahedral virus that has been identified in several avian and mammalian hosts. Human adenoviruses (hAds) belong to the Mastadenovirus genus which includes all known human and many Ads of animal (e. g., bovine, porcine, canine, murine, equine, simian and ovine) origin. Human adenoviruses are generally divided into six subgroups (A-F) based on a number of biological, chemical, immunological and structural criteria which include hemagglutination properties of rat and rhesus monkey erythrocytes, DNA homology, restriction enzyme cleavage patterns, percentage G+C content and oncogenicity (Straus, 1984, in The Adenoviruses, ed. H. Ginsberg, pps. 451-498, New York: Plenus Press, and Horwitz, 1990; in Virology, eds. B. N. Fields and D. M. Knipe, pps. 1679-1721).

The adenoviral virion has an icosahedral symmetry and, depending on the serotype, a diameter of 60-90 nm. The icosahedral capsid comprises three major proteins, hexon (II), penton base (III) and a knobbed fiber (IV) protein (W. C. Russel, J. Gen. Virol., 81: 2573-2604 (2000)). More specifically, the adenoviral capsid comprises 252 capsomeres, of which 240 are hexons and 12 are pentons. The hexons and pentons are derived from three different viral polypeptides. The hexon comprises three identical polypeptides, namely polypeptide II. The penton comprises a penton base, which provides a point of attachment to the capsid, and a trimeric fiber protein, which is noncovalently bound to and projects from the penton base. Other proteins, namely proteins IX, VI, and Ma are usually also present in the adenoviral capsid. These proteins are believed to stabilize the viral capsid.

One aspect of the preexisting immunity that is observed in humans is humoral immunity, which can result in the production and persistence of antibodies that are specific for adenoviral proteins. The humoral response elicited by adenovirus is directed against the capsid. Adenoviruses isolated from non human great apes are closely related to adenoviruses isolated from humans as demonstrated by their efficient propagation in cells of human origin.

The capsid can be modified as described herein by incorporating non-adenoviral polypeptides, such as T- and/or B-cell epitopes.

The term “hexon protein” refers to the hexon (II) protein comprised in an adenovirus. A hexon protein or a variant thereof according to the invention has the same function as a hexon protein or a fragment thereof in an infectious adenovirus virion. Thus, an adenovirus comprising said hexon or variant thereof preferably as a capsid protein is capable of entering a host cell. A suitable method for generating variants of a hexon protein is described in U.S. Pat. No. 5,922,315. In this method, at least one loop region of the adenovirus hexon is changed with at least one loop region of another adenovirus serotype. It can be easily determined if a recombinant adenovirus can enter a host cell. For example, after contacting a host cell with the adenovirus, the recombinant host cell can be washed and lysed and it can be determined whether adenoviral RNA and/or DNA is found in the host cell using, e.g. an appropriate hybridization probe specific for adenoviral RNA and/or DNA. Alternatively or additionally, the host cell after having been brought into contact with the recombinant adenovirus may be washed, lysed and probed with adenovirus specific antibodies, e.g. using a Western blot. In yet another alternative, it is observed, e.g. in vivo, whether the host cell expresses a gene product, for example a fluorescent protein upon infection with a recombinant adenovirus that comprises a suitable expression cassette to express the gene product in the host cell.

By “adenoviral penton protein” is meant the penton base (III) protein comprised in an adenovirus. An adenoviral penton protein is characterized in that it localizes to the corners of the icosahedral symmetry of the capsid. A penton protein or a variant thereof according to the invention has the same function as a penton protein in an infectious adenovirus virion. Thus, an adenovirus comprising said penton or variant thereof preferably as a capsid protein is capable of entering a host cell, which can be tested as described above. Further, a functional penton has an affinity to an adenoviral fiber protein. The average skilled person is well aware of how to test protein-protein affinities. To determine if a first protein is capable of binding a second protein, he may use, for example, a genetic yeast two-hybrid assay or a biochemical assay such as a pull-down, an enzyme-linked immunosorbent assay (ELISA), a fluorescence-activated cell sorting (FACS)-based assay or a Plasmon resonance assay. When using pull-down or Plasmon resonance assays, it is useful to fuse at least one of the proteins to an affinity tag such as HIS-tag, GST-tag or other, as is well known in the art of biochemistry.

The term “fiber protein” refers to the knobbed fiber (IV) protein comprised in an adenovirus. A fiber protein or a variant thereof according to the invention has the same function as a fiber protein or a fragment thereof in an infectious adenovirus virion. Thus, an adenovirus comprising said fiber or fiber variant preferably as a capsid protein is capable of entering a host cell, which can be tested as described above. Further, a functional fiber protein has an affinity to an adenoviral penton protein. Also, a functional adenoviral fiber protein in its glycosylated form is capable of trimerizing. Thus, it is also preferred that the variant is capable of being glycosylated and/or of forming a trimer. Affinity, including trimerization, can be tested as described above, and glycosylation assays are also well-known in the art.

The term “identity” or “identical” in the context of polynucleotide, polypeptide or protein sequences refers to the number of residues in the two sequences that are identical when aligned for maximum correspondence. Specifically, the percent sequence identity of two sequences, whether nucleic acid or amino acid sequences, is the number of exact matches between two aligned sequences divided by the length of the shorter sequence and multiplied by 100. Alignment tools that can be used to align two sequences are well known to the person skilled in the art and can, for example, be obtained on the World Wide Web, e.g., Clustal Omega (http://www.ebi.ac.uk/Tools/msa/clustalo/) for polypeptide alignments or MUSCLE (http://www.ebi.ac.uk/Tools/msa/muscle/) or MAFFT (http://www.ebi.ac.uk/Tool s/m sa/mafft/) for polynucleotide alignments or WATER (http://www.ebi.ac.uk/Tools/psa/emboss water/) for polynucleotide and polypeptide alignments. The alignments between two sequences may be carried out using default parameters settings, e.g. for MAFFT preferably: Matrix: Blosum62, Gap Open 1.53, Gap Extend 0.123, for WATER polynucleotides preferably: MATRIX: DNAFULL, Gap Open: 10.0, Gap Extend 0.5 and for WATER polypeptides preferably MATRIX: BLOSUM62, Gap Open: 10.0, Gap Extend: 0.5. Those skilled in the art understand that it may be necessary to introduce gaps in either sequence to produce a satisfactory alignment. The “best sequence alignment” is defined as the alignment that produces the largest number of aligned identical residues while having a minimal number of gaps. Preferably, it is a global alignment, which includes every residue in every sequence in the alignment.

The term “variant” refers, with respect to a polypeptide, generally to a modified version of the polypeptide, e.g. a mutation, so one or more amino acids of the polypeptide may be deleted, inserted, modified and/or substituted. Generally, the variant is functional, meaning that an adenovirus comprising the functional variant is capable of infecting a host cell. More specific functions are defined herein and have precedence over the general definition. A “mutation” or “amino acid mutation” can be an amino acid substitution, deletion and/or insertion (“and” may apply if there is more than one mutation). Preferably, it is a substitution (i.e. a conservative or non-conservative amino acid substitution), more preferably a conservative amino acid substitution. In some embodiments, a substitution also includes the exchange of a naturally occurring amino acid with a not naturally occurring amino acid. A conservative substitution comprises the substitution of an amino acid with another amino acid having a chemical property similar to the amino acid that is substituted. Preferably, the conservative substitution is a substitution selected from the group consisting of:
(i) a substitution of a basic amino acid with another, different basic amino acid;
(ii) a substitution of an acidic amino acid with another, different acidic amino acid;
(iii) a substitution of an aromatic amino acid with another, different aromatic amino acid;
(iv) a substitution of a non-polar, aliphatic amino acid with another, different non-polar, aliphatic amino acid; and
(v) a substitution of a polar, uncharged amino acid with another, different polar, uncharged amino acid.

A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of glycine, alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

Means for determining sequence identity are described above.

Amino acids of a protein may also be modified, e.g. chemically modified. For example, the side chain or a free amino or carboxy-terminus of an amino acid of the protein or polypeptide may be modified by e.g. glycosylation, amidation, phosphorylation, ubiquitination, etc. The chemical modification can also take place in vivo, e.g. in a host-cell, as is well known in the art. For example, a suitable chemical modification motif, e.g. glycosylation sequence motif present in the amino acid sequence of the protein will cause the protein to be glycosylated. Unless a modification leads to a change in identity of a modified amino acid (e.g. a substitution or deletion), a modified polypeptide is within the scope of polypeptide as mentioned with respect to a certain SEQ ID NO, i.e. it is not a variant as defined herein.

The term “variant” refers, with respect to a polynucleotide, generally to a modified version of the polynucleotide, e.g. a mutation, so one or more nucleotides of the polynucleotide may be deleted, inserted, modified and/or substituted. Generally, the variant is functional, meaning that an adenovirus comprising the functional variant is capable of infecting a host cell. More specific functions are defined herein and have precedence over the general definition. A “mutation” can be a nucleotide substitution, deletion and/or insertion (“and” may apply if there is more than one mutation). Preferably, it is a substitution, more preferably it causes an amino acid substitution, most preferably a conservative amino acid substitution.

An “antigenic protein or fragment thereof” (wherein the fragment is also antigenic) is capable of eliciting an immune response in a mammal. Preferably, it is a tumor antigen or an antigen derived from a pathogen. The term “pathogen” refers to any organism which may cause disease in a subject. It includes but is not limited to bacteria, protozoa, fungi, nematodes, viroids, viruses and parasites, wherein each pathogen is capable, either by itself or in concert with another pathogen, of eliciting disease in vertebrates including but not limited to mammals, and including but not limited to humans. As used herein, the term “pathogen” also encompasses organisms which may not ordinarily be pathogenic in a non-immunocompromised host, but are in an immunocompromised host.

Generally speaking, the adenoviral genome is well characterized. There is general conservation in the overall organization of the adenoviral genome with respect to specific open reading frames being similarly positioned, e.g. the location of the E1A, E1B, E2A, E2B, E3, E4, LI, L2, L3, L4 and L5 genes of each virus. Each extremity of the adenoviral genome comprises a sequence known as an inverted terminal repeat (ITRs), which is necessary for viral replication. The virus also comprises a virus-encoded protease, which is necessary for processing some of the structural proteins required to produce infectious virions. The structure of the adenoviral genome is described on the basis of the order in which the viral genes are expressed following host cell transduction. More specifically, the viral genes are referred to as early (E) or late (L) genes according to whether transcription occurs prior to or after onset of DNA replication. In the early phase of transduction, the E1A, E1B, E2A, E2B, E3 and E4 genes of adenovirus are expressed to prepare the host cell for viral replication. During the late phase of infection, expression of the late genes L1-L5, which encode the structural components of the virus particles are activated.

The term “vector” as used herein includes any vectors known to the skilled person including plasmid vectors, cosmid vectors, phage vectors such as lambda phage, viral vectors such as adenovirus (Ad) vectors (e.g. as exemplified in the further aspect of the invention above), adeno-associated virus (AAV) vectors (e.g., AAV type 5), alphavirus vectors (e.g., Venezuelan equine encephalitis virus (VEE), sindbis virus (SIN), semliki forest virus (SFV), and VEE-SIN chimeras), herpes virus vectors, measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), and vesicular stomatitis virus vectors, viral like particles, or bacterial spores. A vector also includes expression vectors, cloning vectors and vectors that are useful to generate recombinant adenoviruses in host cells.

As stated above, a “heterologous protein or fragment thereof” can be a non-adenoviral protein or fragment thereof, in particular an antigenic protein or fragment thereof. To this end, the polynucleotide encoding a heterologous protein may be a molecule to be delivery into a target cell, e.g. a polynucleotide encoding an antigenic protein or a fragment thereof, preferably an antigenic protein or a fragment of a pathogen such as a pathogenic virus, bacterium, fungus, protozoan or parasite, or a tumour antigen. “Antigen” refers to any protein or peptide capable of eliciting an immune response in a mammal. An antigen comprises preferably at least 8 amino acids and most preferably comprises between 8 and 12 amino acids.

The term “expression cassette” refers to a nucleic acid molecule which comprises at least one nucleic acid sequence that is to be expressed, along with its transcription and translation control sequences. Changing the expression cassette will cause the vector in which it is incorporated to direct the expression of a different sequence or combination of sequences. Because of the restriction sites being preferably engineered to be present at the 5′ and 3′ ends, the cassette can be easily inserted, removed, or replaced with another cassette. Preferably, an expression cassette includes cis-regulating elements for efficient expression of a given gene, such as promoter, initiation-site and/or polyadenylation-site. More specific with respect to the present invention, an expression cassette contains all the additional elements required for the expression of the polynucleotide of the first aspect in host cells. A typical expression cassette thus contains a promoter operatively linked to the polynucleotide of the first aspect and signals required for efficient polyadenylation of the transcript, ribosome binding sites, and translation termination. Additional elements of the cassette may include, for example enhancers. An expression cassette should also contain a transcription termination region downstream of the structural gene to provide for efficient termination. The termination region may be obtained from the same gene as the promoter sequence or may be obtained from different genes.

As used herein, the term “minigene” refers to a heterologous gene construct wherein one or more functionally nonessential segments of a gene are deleted with respect to the naturally occurring gene. A “minigene cassette” is an expression cassette comprising a minigene for expression.

The term “replication-competent” recombinant adenovirus (AdV) refers to an adenovirus which can replicate in a host cell in the absence of any recombinant helper proteins comprised in the cell. Preferably, a “replication-competent” adenovirus comprises the following intact or functional essential early genes: E1A, E1B, E2A, E2B, E3 and E4. Wild type adenoviruses isolated from a particular animal will be replication competent in that animal.

The term “replication-defective” or “replication-incompetent” recombinant adenovirus refers to an adenovirus that has been rendered to be incapable of replication because it has been engineered to comprise at least a functional deletion, i.e. a deletion which impairs the function of a gene without removing it entirely, e.g. introduction of artificial stop codons, deletion or mutation of active sites or interaction domains, mutation or deletion of a regulatory sequence of a gene etc., or a complete removal of a gene encoding a gene product that is essential for viral replication, such as one or more of the adenoviral genes selected from E1, E2, E3 and E4. The recombinant adenoviral viruses of the invention are preferably replication-defective.

The term “recombinant adenovirus” refers in particular to an adenovirus that is modified to comprise a heterologous polynucleotide and/or polypeptide sequence. “Heterologous” can mean from another adenovirus strain, in particular a strain from a different host (e.g. a human host, so from a human adenovirus such as Ad3 or Ad5), or from a non-adenoviral organism such an antigen derived from a pathogen as described herein, or from human such as a human tumor antigen. As such, the term comprises chimeric and carrier adenoviruses, respectively. A recombinant adenovirus can comprise a heterologous polynucleotide and/or polypeptide sequence from both other adenoviruses or from non-adenoviral organisms, i.e. it can be both a chimeric and a carrier adenovirus.

As used herein, the term “virus-like particle” or “VLP” refers to a non-replicating, empty viral shell, derived in this case from an adenovirus. VLPs are generally composed of one or more viral proteins, such as, but not limited to those proteins referred to as capsid, coat, shell, surface and/or envelope proteins. They contain functional viral proteins responsible for cell penetration by the virus, which ensures efficient cell entry. VLPs can form spontaneously upon recombinant expression of the protein in an appropriate expression system. Methods for producing particular VLPs are known in the art. Adenovirus VLPs in particular can be produced by functionally impairing, e.g. deleting or introducing a null-mutation into the Iva2 gene of an adenovirus, which is involved in viral DNA packing (Ostapchuk et al. J Virol. 2011 June; 85(11): 5524-5531). The presence of VLPs can be detected using conventional techniques known in the art, such as by electron microscopy, X-ray crystallography, and the like. See, e.g., Baker et al., Biophys. J. (1991) 60:1445-1456; Hagensee et al., J. Virol. (1994) 68:4503-4505. For example, cryoelectron microscopy can be performed on vitrified aqueous samples of the VLP preparation in question, and images recorded under appropriate exposure conditions.

“Substantially no adenoviral genomic DNA” comprised in a VLP means that there is either no such genomic DNA in the VLP or not sufficient DNA in the VLP to allow virus replication in a cell infected with the VLP and not expressing DNA that would complement the DNA in the VLP such that virus replication can occur.

Further to the above, an “epitope”, also known as antigenic determinant, is the segment of a macromolecule that is recognized by the immune system, specifically by antibodies, B cells, or T cells. In the context of the present invention it is preferred that the term “epitope” refers to the segment of protein or polyprotein that is recognized by the immune system. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

A “non-adenoviral T-cell epitope” is an epitope that can be presented on the surface of an antigen-presenting cell, where it is bound to an MHC molecule. In humans, professional antigen-presenting cells are specialized to present MHC class II peptides, whereas most nucleated somatic cells present MHC class I peptides. T-cell epitopes presented by MHC class I molecules are typically peptides between 8 and 11 amino acids in length, whereas MHC class II molecules present longer peptides, 13-17 amino acids in length.

A “non-adenoviral B-cell epitope” is an epitope that is recognised as three-dimensional structures on the surface of native antigens by B-cells.

B- and T-cell epitopes can be predicted with in silico tools, e.g. the online B- or T-cell prediction tools of the IEDB Analysis Resource.

The term “presents one or more non-adenoviral B-cell epitopes” means that the one or more epitopes are incorporated into the capsid such that they be recognized by B-cells. The term “incorporates one or more non-adenoviral B-/T-cell epitopes” means that the epitope is either contained in the VLP without being incorporated in the capsid, or is incorporated in the capsid. If it is incorporated in the capsid, it may or may not be presented to the outside such that it can be recognized by immune cells.

An “immunological adjuvant” or simply “adjuvant” is a substance that accelerates, prolongs and/or enhances the quality and/or strength of an immune response to an antigen/immunogen, in comparison to the administration of the antigen alone, thus, reducing the quantity of antigen/immunogen necessary in any given vaccine, and/or the frequency of injection necessary in order to generate an adequate immune response to the antigen/immunogen of interest. Examples of adjuvants that may be used in the context of the composition according to the present invention are gel-like precipitates of aluminum hydroxide (alum); AlPO4; alhydrogel; bacterial products from the outer membrane of Gram-negative bacteria, in particular monophosphoryl lipid A (MPLA), lipopolysaccharides (LPS), muramyl dipeptides and derivatives thereof; Freund's incomplete adjuvant; liposomes, in particular neutral liposomes, liposomes containing the composition and optionally cytokines; non-ionic block copolymers; ISCOMATRIX adjuvant (Drane et al., 2007); unmethylated DNA comprising CpG dinucleotides (CpG motif), in particular CpG ODN with a phosphorothioate (PTO) backbone (CpG PTO ODN) or phosphodiester (PO) backbone (CpG PO ODN); synthetic lipopeptide derivatives, in particular Pam3Cys; lipoarabinomannan; peptidoglycan; zymosan; heat shock proteins (HSP), in particular HSP 70; dsRNA and synthetic derivatives thereof, in particular Poly I:poly C; polycationic peptides, in particular poly-L-arginine; taxol; fibronectin; flagellin; imidazoquinoline; cytokines with adjuvant activity, in particular GM-CSF, interleukin- (IL-)2, IL-6, IL-7, IL-18, type I and II interferons, in particular interferon-gamma, TNF-alpha; 25-dihydroxyvitamin D3 (calcitriol); and synthetic oligopeptides, in particular MHCII-presented peptides. Non-ionic block polymers containing polyoxyethylene (POE) and polyoxypropylene (POP), such as POE-POP-POE block copolymers may be used as an adjuvant (Newman et al., 1998). This type of adjuvant is particularly useful for compositions comprising nucleic acids as active ingredient.

The term “vaccination” in the context of the present invention is an active immunization, that is an induction of a specific immune response by administering (for example, subcutaneously, intradermally, intramuscularly, orally, nasally) of an antigen (a substance that is recognized as foreign by the immune system of the vaccinated individual and is immunogenic) in a suitable immunogenic formulation. The antigen is thus used as a trigger for the immune system to build up a specific immune response to the antigen. A vaccination within the scope of the present invention can in principle be carried out both in the therapeutic sense, but also in the prophylactic sense. It includes vaccination against pathogens as described herein to treat or prevent infectious diseases, or vaccination to treat or prevent non-infectious diseases, such as cancer. In case of non-infectious diseases, the antigen is preferably a cellular membrane antigen, in particular one that is expressed only by a diseased cell, but not by non-diseased cells. An example is a tumor-associated antigen. In this context, the term “tumor-associated antigen” means a structure which is predominantly presented by tumor cells and thereby allows a differentiation from non-malignant tissue. Preferably, such a tumor-associated antigen is located on or in the cell membrane of a tumor cell. Examples of tumor-associated antigens are described, e.g., in DeVita et al. (Eds., “Biological Therapy of Cancer”, 2. Edition, Chapter 3: Biology of Tumor Antigens, Lippincott Company, ISBN 0-397-51416-6 (1995)).

“Priming” as used herein refers to the administration of a vaccine for inducing/generating an immune response in a mammal, and “boosting” to the administration of a vaccine for enhancing an immune response in a mammal. The phrase “heterologous prime-boost” means that the vaccine for inducing/generating an immune response (priming) in a mammal and the vaccine for enhancing the immune response (boosting) in a mammal are different. Heterologous prime-boost is useful if a subject, e.g. patient has developed antibodies against a first vector and a boosting is required. In this context, a first (prime) and a second (boost) vaccine, e.g. adenovirus, are sufficiently different, if the antibody response induced during priming by the first vaccine does not prevent more than 70% or preferably more than 80% of the second vaccine particles administered for boosting from entering the nucleus of cells of the animal that has been subjected to priming and boosting.

The term “gene therapy” can be broadly defined as the concept of directed introduction of foreign genetic material into a cell, tissue or organ for correction of defective genes with the goal to improve the clinical status of a patient. As used herein, the term “gene therapy” preferably refers to “somatic therapy” and not to “germ line therapy”, which would induce heritable changes passed from generation to generation, wherein the somatic therapy restricts the therapeutic effect to the treated individual. The gene therapy, preferably the somatic therapy, can be further discriminated by a fast and easy to perform direct gene transfer to the organism (“in vivo”) or a sophisticated but more specific and controllable gene transfer to explanted cells or tissues (“ex vivo” or “in vitro”), which are re-implanted after treatment.

The term “neutralizing antibody” refers to an antibody that binds to an epitope of the adenovirus and prevents it from producing a productive infection in a host cell or prevents the transduction of a target cell with a replication incompentent vector expressing a transgene, e.g. the adenovirus DNA is capable of entering a cell, in particular a host cell.

The term “SARS CoV-2”, “SARS-COV2”, “SARS-CoV-2”, “Severe acute respiratory syndrome coronavirus 2” and “2019-nCoV” are used interchangeably throughout and refer to the virus causing the coronavirus disease 2019 (COVID-2019 or COVID-19).

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The invention is described by way of the following examples which are to be construed as merely illustrative and not limitative of the scope of the invention.

### EXAMPLES

### Example 1: GRAd32, GRAd23 and GRAd21

The construction of pGRAd vectors proceeded through the steps detailed below. The pGRAd32, pGRAd23 and GRAd21 vectors are derived from wild type Adenovirus strains isolated from stool samples obtained from healthy gorilla using standard procedures. Wild type viruses were isolated by inoculating monolayers of A549 cell with stool extracts. Cell monolayers were observed daily for the appearance of cytopathic effect. Samples scored positive by observation under the microscope were harvested and the cells lysed by freeze-thaw (−80° C./37° C.). The clarified cell lysates were then used for virus propagation by infecting monolayers of fresh cells. After two passages of virus amplification, the adenoviruses were then purified by using standard procedures.

The viral genomes (GRAd32, SEQ ID NO: 1; GRAd23, SEQ ID NO: 22; GRAd21, SEQ ID NO: 10) were extracted from purified viruses by SDS/proteinase K digestion followed by phenol-chloroform extraction. The purified adenovirus DNA were cloned in a shuttle plasmid vector to be further modified by introducing the following deletions of viral genome: GRAd32:
1) deletion of the E1 region (from bp 445 to bp 3403) of the viral genome
2) deletion of the E3 region (from bp 28479 to bp 32001) of the viral genome
3) deletion of the E4 region (from bp 34144 to bp 36821) of the viral genome.

GRAd23:
1) deletion of the E1 region (from bp 451 to bp 3403) of the viral genome
2) deletion of the E3 region (from bp 28494 to bp 32016) of the viral genome
3) deletion of the E4 region (from bp 34159 to bp 36836) of the viral genome.

GRAd21:
1) deletion of the E1 region (from bp 456 to bp 3403) of the viral genome
2) deletion of the E3 region (from bp 28343 to bp 31875) of the viral genome
3) deletion of the E4 region (from bp 34005 to bp 36681) of the viral genome

GRAd Shuttle Vector

A Gorilla Group C Adenovirus shuttle vector was constructed according to the following steps:

The first step was the construction of the plasmid pGRAd ITRs-only shuttle: GRAd left end was amplified by PCR using the plasmid “pUC57-GRAd ends” (SEQ ID NO: 34) as template, and the primers below:

(SEQ ID NO: 70)
Fw: 5′-cca ggc cgt gcc ggc acg ttc-3′
(SEQ ID NO: 71)
Rev: 5′-att acc ctg tta tcc cta cgt c-3′

GRAd right end was amplified by PCR using the plasmid “pUC57-GRAd ends” (SEQ ID NO: 34) as template, and the primers below:

(SEQ ID NO: 72)
Fw: 5′-gta ggg ata aca ggg taa tgc a-3′
(SEQ ID NO: 73)
Rev: 5′-aaa cat gag aat tgg tcg acg g-3′

GRAd left end and GRAd right end were cloned according to Gibson assembly method into pBeloBAC11 (SEQ ID NO: 35) previously digested with HpaI/SfiI to obtain “pGRAd ITRs-only shuttle” (SEQ ID NO: 36).

The second step was the construction of the plasmid “pDE1_GRAd_shuttle”:

The hCMVtetO-GAG-bGHpolyA cassette was amplified by PCR using the plasmid “phCMVtetO-GAG-bGHpolyA” (SEQ ID NO: 37), Gag antigen encoded by nucleotides 1220 to 2719 of SEQ ID NO: 37, as template, and the primers below:

(SEQ ID NO: 74)
Fw: 5′-gtt ttt att gtc gcc gtc atc tga cgg gcc
gcc att gca tac gtt gta tcc ata tc-3′
(SEQ ID NO: 75)
Rev: 5′-aag cgc gat cgc ggc cgc ggc cat aga gcc
cac cgc atc c-3′

GRAd fragment containg pIX coding region was amplified by PCR using the plasmid “pGRAd pIX” (SEQ ID NO: 38) as template, and the primers below:

(SEQ ID NO: 76)
Fw: 5′-ccg cgg ccg cga tcg cgc tta ggc ctg acc
atc tgg-3′
(SEQ ID NO: 77)
Rev: 5′-ctg tta tcc cta ggc gcg cct tag ggg gag
gca agg ctg-3′

The Amp-LacZ-SacB selection cassette was amplified by PCR using the plasmid “pAmpR-LacZ-SacB” (SEQ ID NO: 39) as template, and the primers below:

(SEQ ID NO: 78)
Fw: 5′-ggc gcg cct agg gat aac agg gta ata ccc
cta ttt gtt tat ttt tct aa-3′
(SEQ ID NO: 79)
Rev: 5′-tgc tgg tgc tgt gag agt gcg act cgg gtc
tag gcg cgc cat tac cct gtt atc cct att att tgt
taa ctg tta att gt-3′

The hCMVtetO::GAG-bGHpolyA cassette, the fragment containing pIX and the AmpR-LacZ-SacB selection cassette were cloned using Gibson assembly method into the “pITRs-only GRAd shuttle” previously digested with I-SceI, to generate “pDE1 GRAd shuttle” (SEQ ID NO: 40).

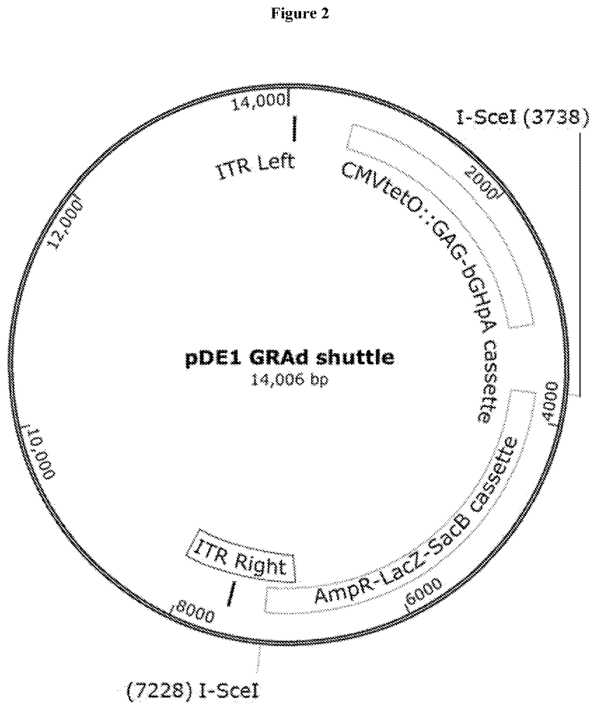

The shuttle plasmid was designed to contain restriction enzyme sites (PmeI) that are present only at the end of both ITRs to allow the release of viral DNA from plasmid DNA. See FIG. 2 for a schematic representation.

### Example 2: GRAd23 Vector Construction

GRAd23 DE1 Vector

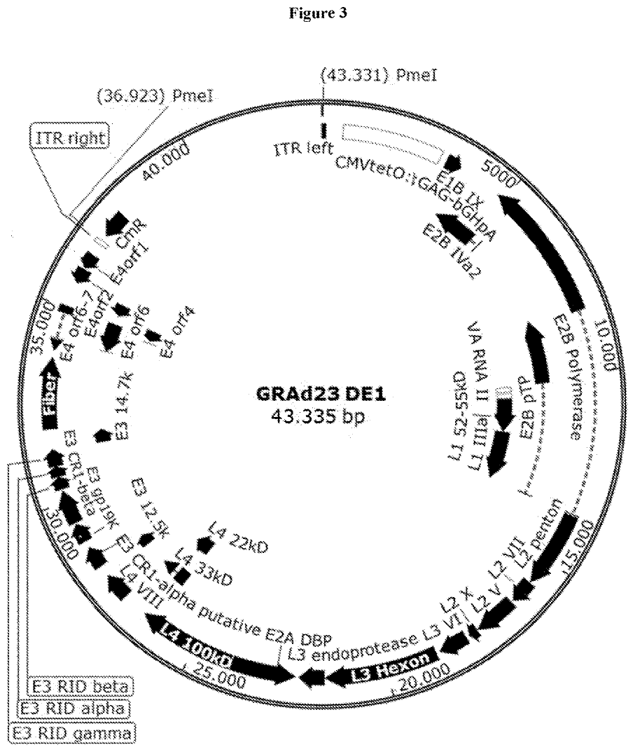

GRAd23 wt genomic DNA (SEQ ID NO: 22) was isolated by Proteinase K digestion followed by phenol/chloroform extraction and then inserted into the pDE1 GRAd shuttle by homologous recombination in E. coli strain BJ5138 to obtain the pGRAd23 vector. Homologous recombination between the pIX gene, the right ITR DNA sequence present at the ends of shuttle (digested with I-SceI) and the viral genomic DNA allowed its insertion into the shuttle vector, deleting at the same time the E1 region that was substituted by the expression cassette, thus finally generating the “pGRAd23 DE1 GAG” BAC vector (SEQ ID NO: 41). A schematic representation of pGRAd23 DE1 GAG BAC is shown in FIG. 3.

GRAd23 DE1 Leftward Vector

The construction strategy was based on two different steps:

First Step: Substitution of the E1 Region with AmpR-LacZ-SacB Selection Cassette

The AmpR-LacZ-SacB selection cassette was amplified by PCR using the plasmid “pAmpR-LacZ-SacB” (SEQ ID NO: 39) as template, and the primers below:

(SEQ ID NO: 80)
Fw: 5′-gtt ccg ggt caa agt ctc cgt ttt tat tgt
cgc cgt cat ctg acg ggc cga ccc cta ttt gtt tat
ttt tct aa-3′
(SEQ ID NO: 81)
Rev: 5′-tgg tgc agg cca gca cca gat ggt cag gcc
taa gcg cga tcg cgg ccc ggt tat ttg tta
act gtt aat tgt cc-3′

The DNA fragment obtained by PCR was then cloned in “pGRAd23 DE1 GAG” BAC (SEQ ID NO: 41) by recombineering, obtaining “pGRAd23 DE1 A/L/S” BAC (SEQ ID NO: 42).

Second step: Deletion of AmpR-LacZ-SacB selection cassette and insertion of hCMVtetO::GAG-bGHpA in E1 in the leftward orientation

The hCMVtetO-GAG-bGHpolyA cassette was amplified by PCR using the plasmid “phCMVtetO-GAG-bGHpolyA” (SEQ ID NO: 37) as template, and the primers below:

(SEQ ID NO: 82)
Fw: 5′-gtt ccg ggt caa agt ctc cgt ttt tat tgt
cgc cgt cat ctg acg ggc cgc cat aga gcc cac
cgc atc-3′
(SEQ ID NO: 83)
Rev: 5′-tgg tgc agg cca gca cca gat ggt cag gcc
taa gcg cga tcg cgg ccc ggc cat tgc ata
cgt tgt atc cat -3′

The DNA fragment obtained by PCR was then cloned in “pGRAd23 DE1 A/L/S” BAC (SEQ ID NO: 42) by recombineering, obtaining “pGRAd23 DE1L GAG” BAC (SEQ ID NO: 43).

GRAd23 DE1DE3 Vector

The construction strategy was based on two different steps;

First step—Substitution of E3 region with AmpR-LacZ-SacB selection cassette:

The AmpR-LacZ-SacB selection cassette was amplified by PCR using the plasmid “pAmp-LacZ-SacB” (SEQ ID NO: 39) as template, and the primers below:

(SEQ ID NO: 84)
Fw: 5′-ctg tca ttt gtg tgc tga gta taa taa agg
ctg aga tca gaa tct act cga ccc cta ttt gtt tat
ttt tct aa-3′
(SEQ ID NO: 85)
Rev: 5′-agt gat ttt tta ttg att aca gtt atg atc
aat tga aag gga taa ggt ctt att tgt taa ctg tta
att gtc c-3′

The DNA fragment obtained by PCR was then inserted in “pGRAd23 DE1” BAC (SEQ ID NO: 41) by recombineering, obtaining “pGRAd23 DE1 GAG DE3 A/L/S” BAC (SEQ ID NO: 44).

Second Step—E3 Region Deletion:

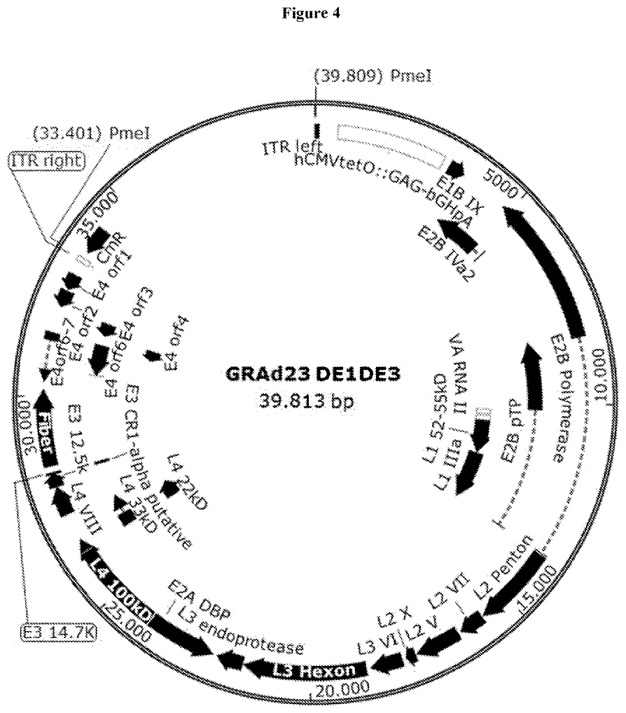

The AmpR-LacZ-SacB selection cassette was deleted using the single strand oligonucleotide 5′-ctg tca ttt gtg tgc tga gta taa taa agg ctg aga tca gaa tct act cgg acc tta tcc ctt tca att gat cat aac tgt aat caa taa aaa atc act-3′ (SEQ ID NO: 86). The single strand DNA fragment oligo was used to replace the selection cassette into “pGRAd23 DE1 GAG DE3 A/L/S” BAC (SEQ ID NO: 44) by recombineering, generating “pGRAd23 DE1 GAG DE3” BAC (SEQ ID NO: 45). This method produced a deletion of the E3 region from bp 28494 to bp 32016 of the GRAd 23 wild type genome. A schematic representation is shown in FIG. 4.

E1E4-Deleted GRAd23 Vector

The construction of GRAd23 vector backbone that includes the deletion of the native E4 region and its substitution with Ad5 E4 orf6 coding region strategy was based on two different steps:

First Step—Substitution of E4 Region with AmpR-LacZ-SacB Selection Cassette:

The AmpR-LacZ-SacB selection cassette was amplified by PCR using the plasmid “pAmpR-LacZ-SacB” (SEQ ID NO: 39) as template, and the primers below:

(SEQ ID NO: 87)
Fw: 5′-ccc ttc cac ata gct taa att atc acc agt
gca aat gga aaa aaa atc aaa ccc cta ttt gtt
tat ttt tct aa-3′
(SEQ ID NO: 88)
Rev: 5′-cgg cac ttg gcc ttt ttc aca ctc tga tta
gtg ctg gtg ctg tga gag tgt tat ttg tta act gtt
aat tgt cc-3′

The DNA fragment obtained by PCR was then inserted by replacing native GRAd23 E4 region in “pGRAd23 DE1 GAG” (SEQ ID NO: 41) BAC by recombineering, obtaining “pGRAd23 DE1 GAG DE4 A/L/S” BAC (SEQ ID NO: 46).

Second Step—Deletion of AmpR-LacZ-SacB Selection Cassette for E4 Region Deletion:

The AmpR-LacZ-SacB selection cassette was deleted, and replaced by the human Adenovirus 5 E4orf6, which was amplified by PCR using the genome of purified wild type human Adenovirus 5 (SEQ ID NO: 47) as template, and the primers below:

(SEQ ID NO: 89)
Fw: 5′-ccc ttc cac ata gct taa att atc acc agt
gca aat gga aaa aaa atc aac tac atg ggg gta
gag tca ta-3′
(SEQ ID NO: 90)
Rev: 5′-cgg cac ttg gcc ttt ttc aca ctc tga tta
gtg ctg gtg ctg tga gag tga tga ctac gtc cgg
cgt tcc-3′

The DNA fragment containing human Ad5 E4 orf6 coding region obtained by PCR was then inserted in “pGRAd23 DE1 GAG DE4 A/L/S” BAC (SEQ ID NO: 46) replacing the AmpR-LacZ-SacB selection cassette by recombineering. The final result was “pGRAd23 DE1 DE4 hAd5E4orf6” BAC (SEQ ID NO: 48)

E1E3E4-Deleted GRAd23 Vector

The construction of GRAd23 vector backbone that includes both the deletion of the E3 region and the deletion of the native E4 region and its substitution with Ad5 E4 orf6 coding region strategy was based on two different steps:

First Step—Substitution of E3 Region with Amp-LacZ-SacB Selection Cassette:

The AmpR-LacZ-SacB selection cassette was amplified by PCR using the plasmid “pAmpR-LacZ-SacB” (SEQ ID NO: 39) as template, and the primers below:

(SEQ ID NO: 91)
Fw: 5′-ctg tca ttt gtg tgc tga gta taa taa agg
ctg aga tca gaa tct act cga ccc cta ttt gtt tat
ttt tct aa-3′
(SEQ ID NO: 92)
Rev: 5′-agt gat ttt tta ttg att aca gtt atg atc
aat tga aag gga taa ggt ctt att tgt taa ctg tta
att gtc c-3′

The DNA fragment obtained by PCR was then inserted in “pGRAd23 DE1 DE4 hAd5E4 orf6” (SEQ ID NO: 48) BAC by recombineering, obtaining “pGRAd23 DE1 GAG DE3 A/L/S DE4 hAd5 E4orf6” BAC (SEQ ID NO: 49).

Second Step— E3 Region Deletion:

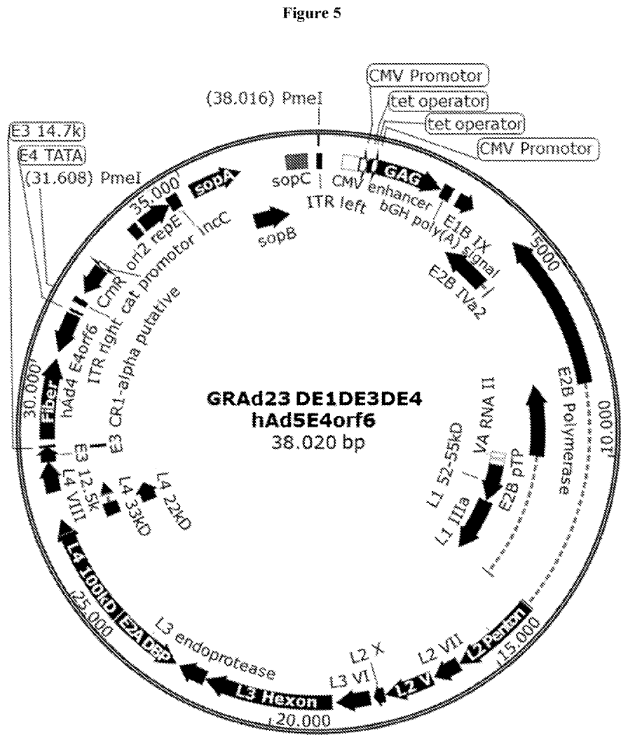

The AmpR-LacZ-SacB selection cassette was deleted using the single strand oligonucleotide 5′-ctg tca ttt gtg tgc tga gta taa taa agg ctg aga tca gaa tct act cgg acc tta tcc ctt tca att gat cat aac tgt aat caa taa aaa atc act-3′ (SEQ ID NO: 86). The single strand DNA fragment oligo was used to replace the selection cassette into “pGRAd23 DE1 GAG DE3 A/L/S DE4 hAd5 E4orf6” BAC (SEQ ID NO: 49) by recombineering, generating “pGRAd23 DE1 GAG DE3 DE4 hAd5 E4orf6” BAC (SEQ ID NO: 50). A schematic representation is shown in FIG. 5.

### Example 3: Construction of GRAd23 Vectors Expressing SARS-CoV2 Spike Gene

The construction of pGRAd23 SARS CoV-2 Spike vector proceeded through the steps outlined below.

Generation of the phCMV-IntronA::I-SceI-WPRE-bGHpA

At first, a pCMV-IntronA::I-SceI-WPRE-bGHpA shuttle plasmid was generated, by modifying a “pUC19-hCMVtetO::SEAP-bGHpA” plasmid (SEQ ID NO: 51).

The Intron A—I-SceI cassette was amplified by PCR using the “pVIJnsA” plasmid (SEQ ID NO: 39) as template, and the primers below:

(SEQ ID NO: 93)
Fw: 5′-acc ggg acc gat cca gcc-3′
(SEQ ID NO: 94)
Rev1: 5′-taa tcc aga ggt tga tta tta ccc tgt tat
ccc tag aat tct ttg cca aaa tga tgc tgc aga aaa
gac cca tgg aa-3′
(SEQ ID NO: 95)
Rev2: 5′-caa att ttg taa tcc aga ggt tga ttc ccg
ggt aat cca gag gtt gat tat tac c-3′

This PCR was performed with a single forward primer and two reverse primers, in order to accommodate space for the insertion of the I-SceI tag in the reverse primer.

The WPRE cassette was amplified by PCR using the plasmid pCAG21 (SEQ ID NO: 53) as template, and the primers below:

(SEQ ID NO: 96)
Fw: 5′-caa cct ctg gat tac aaa att tg-3′
(SEQ ID NO: 97)
Rev: 5′-acg cgg gga cca cgg gtt aac ccg ggg cgg
gga ggc ggc cca aa-3′

The IntronA-I-SceI PCR product and the WPRE cassette PCR product were ligated according to Gibson method into the “pUC19-hCMVtetO::SEAP-bGHpA” plasmid (SEQ ID NO: 51) previously digested with HindIII-Sural, generating “phCMVtetO-IntronA::I-SceI-WPRE-bGHpA” (SEQ ID NO: 54)

Generation of the phCMV-IntronA::SARS CoV-2 S-WPRE-bGHpA

The full coding sequence of the surface glycoprotein S (Genbank Accession No. QHD43416 identical to YP_009724390) of the SARS CoV-2 virus (Genbank Accession NC_045512.2 identical to MN908947) was chemically synthesized by Doulix (Via Torino, 107, 30172 Venezia VE) changing codon usage, including a minimal Kozak sequence upstream the first ATG and fusing the human influenza hemagglutinin (HA) TAG coding sequence to the 3′ end of the S gene (SEQ ID NO: 29): Kozak: nucleotide 1 to 5, spike protein nucleotide 6 to 3824, HA TAG nucleotide 3825 to 3857, stop codon nucleotide 3858 to 3860. The modified S gene was cloned by Doulix by Gibson assembly method into the I-SceI site of the “pCMV-IntronA::I-SceI-WPRE-bGHpA” (SEQ ID NO: 54), generating the plasmid “phCMVtetO-IntronA::SARS CoV-2 S-WPRE-bGHpA” (SEQ ID NO: 55).

Construction of the DE1L DE3 GRAd23 SARS CoV-2 S

The insertion of the SARS CoV-2 S gene expression cassette into the DE1L DE3 deleted GRAd23 vector in the leftward orientation was obtained through the following steps:

First Step—Substitution of E3 Region with AmpR-LacZ-SacB Selection Cassette in a DE1L Backbone:

The AmpR-LacZ-SacB selection cassette was amplified by PCR using the plasmid “pAmpR-LacZ-SacB” (SEQ ID NO: 39) as template, and the primers below:

(SEQ ID NO: 98)
Fw: 5′-ctg tca ttt gtg tgc tga gta taa taa agg
tat ttt tct ctg aga tca gaa tct act cga ccc cta
ttt gtt aa-3′
(SEQ ID NO: 99)
Rev: 5′-agt gat ttt tta ttg att aca gtt atg atc
aat tga aag gga taa ggt ctt att tgt taa ctg tta
att gtc c-3′

The DNA fragment obtained by PCR was then inserted in “pGRAd23 DE1L GAG” BAC (SEQ ID NO: 43) by recombineering, obtaining “pGRAd23 DE1L GAG DE3 A/L/S” BAC (SEQ ID NO: 56).

Second Step—E3 Region Deletion:

The AmpR-LacZ-SacB selection cassette was deleted using the single strand oligonucleotide 5′-ctg tca ttt gtg tgc tga gta taa taa agg ctg aga tca gaa tct act cgg acc tta tcc ctt tca att gat cat aac tgt aat caa taa aaa atc act-3′ (SEQ ID NO: 86). The single strand DNA fragment oligo was used to replace the selection cassette into “pGRAd23 DE1L GAG DE3 A/L/S” BAC (SEQ ID NO: 56) by recombineering generating “pGRAd23 DE1L GAG DE3” BAC (SEQ ID NO: 57).

Third step—Substitution of the leftward GAG region with Amp-LacZ-SacB selection cassette: The AmpR-LacZ-SacB selection cassette was amplified by PCR using the plasmid “pAmpR-LacZ-SacB (SEQ ID NO: 39) as template and the primers below:

(SEQ ID NO: 100)
Fw: 5′-gat ggc tgg caa cta gaa ggc aca gca gat
cgc ggc cgc tgt cga ctg aat tct gat ggg ctt tat
ttt att att tgt taa ctg tta att gtc-3′
(SEQ ID NO: 101)
Rev: 5′-cga tcc agc ctc cgc ggc cgg gaa cgg tgc
att gga acg cgg att ccc cgt gcc aag agt gag atc
tac cac ccc tat ttg ttt att ttt ct-3′

The DNA fragment obtained by PCR was then cloned in “pGRAd23 DE1L GAG DE3” BAC (SEQ ID NO: 57) by recombineering, obtaining “pGRAd23 DE1L A/L/S DE3” BAC (SEQ ID NO: 27).

Fourth step—Deletion of AmpR-LacZ-SacB selection cassette for replacement with the hCMVtetO-IntronA::SARS-CoV-2 S-WPRE-bGHpA in E1 in the leftward orientation: The full cassette hCMVtetO-IntronA::kozak-SARS CoV-2 S-HA-WPRE-bGHpA cassette was retrieved by SpeI/PacI digestion from the plasmid “phCMVtetO-IntronA::SARS CoV-2 S-WPRE-bGHpA” (SEQ ID NO: 55) and cloned into the “pGRAd23 DE1L A/L/S DE3” BAC (SEQ ID NO: 27), generating pGRAd23 DE1L hCMVtetO-IntronA::SARS CoV-2 S-WPRE-bGHpA DE3″ BAC. (SEQ ID NO: 32).

### Example 4: GRAd32 Vector Construction

Construction of GRAd32 DE1 Vector

GRAd32 wt genomic DNA (SEQ ID NO: 1) was isolated by Proteinase K digestion followed by phenol/chloroform extraction and then inserted in pDE1 GRAd shuttle (SEQ ID NO: 40) by homologous recombination in the E. coli strain BJ5138 to obtain pGRAd32 vector. Homologous recombination between the pIX gene, the right ITR DNA sequences present at the ends of shuttle (digested with I-SceI) and the viral genomic DNA allowed its insertion in the shuttle vector, by deleting at the same time the E1 region that was substituted by the GAG expression cassette, finally generating the “pGRAd32 DE1 GAG wrongITR-L” BAC vector (SEQ ID NO: 58), which retained the pIX and the right ITR of the GRAd32, and the left ITR of the shuttle BAC.

Correction of GRAd32 DE1 Vector ITR-L

The construction strategy was based on two different steps as described below:

First Step: Substitution of the ITR-L Region with AmpR-LacZ-SacB Selection Cassette

The AmpR-LacZ-SacB selection cassette was amplified by PCR using the plasmid “pAmpR-LacZ-SacB” (SEQ ID NO: 39) as template, and the primers below:

(SEQ ID NO: 102)
Fw: 5′-tgt cct gct tat cca caa cat ttt gcg cac
ggt tat gtg gac aaa ata cct ggt tac ccc tat ttg
ttt att ttt ct-3′
(SEQ ID NO: 103)
Rev: 5′-gac atg agc caa tat aaa tgta cat att atg
ata tgg ata caa cgt atg caa tgg tta ttt gtt aac
tgt taa ttg tc-3′

The DNA fragment obtained by PCR was then cloned in “pGRAd32 DE1 GAG wrongITR-L” BAC (SEQ ID NO: 58) by recombineering, obtaining “pGRAd23 DE1 GAG wrongITR-L ALS in ITR-L” BAC (SEQ ID NO: 59).

Second Step: Deletion of AmpR-LacZ-SacB Selection Cassette and Insertion of the Correct ITR-L:

The ITR-L was amplified by PCR using the GRAd32 genomic DNA (SEQ ID NO: 1) as template, and the primers below:

(SEQ ID NO: 104)
Fw: 5′-tgt cct gct tat cca caa cat ttt gcg cac
ggt tat gtg gac aaa ata cct ggt tgc cgt tta aac
cat cat caa taa tat acc tta ttt tg-3′
(SEQ ID NO: 105)
Rev: 5′-gac atg agc caa tat aaa tgt aca tat tat
gat atg gat aca acg tat gca atg gcg gcc atg acg
gtg aca ata aaa acg ga-3′.

The DNA fragment obtained by PCR was then cloned in “pGRAd23 DE1 GAG wrongITR-L ALS in ITR-L” BAC (SEQ ID NO: 59) by recombineering, obtaining “pGRAd23 DE1 GAG” ITRs corrected BAC (SEQ ID NO: 60).

Construction of GRAd32 DE3DE4 Vector

The construction strategy was based on four different steps as described below:

First Step—Substitution of E3 Region with AmpR-LacZ-SacB Selection Cassette:

The AmpR-LacZ-SacB selection cassette was amplified by PCR using the plasmid “pAmp-LacZ-SacB” (SEQ ID NO: 39) as template, and the primers below:

(SEQ ID NO: 106)
Fw: 5′-ctg tca ttt gtg tgc tga gta taa taa agg
ttt tct ctg aga tca gaa tct act cga ccc cta ttt
gtt tat aa-3′
(SEQ ID NO: 107)
Rev: 5′-agt gat ttt tta ttg att aca gtt atg atc
aat tga aag gga taa ggt ctt att tgt taa ctg tta
att gtc c-3′.

The DNA fragment obtained by PCR was then inserted in “pGRAd32 DE1 GAG” BAC (SEQ ID NO: 60) by recombineering, obtaining “pGRAd32 DE1 GAG DE3 ALS” BAC (SEQ ID NO: 61).

Second Step—E3 Region Deletion:

The AmpR-LacZ-SacB selection cassette was deleted using the single strand oligonucleotide 5′-ctg tca ttt gtg tgc tga gta taa taa agg ctg aga tca gaa tct act cgg acc tta tcc ctt tca att gat cat aac tgt aat caa taa aaa atc act-3′ (SEQ ID NO: 86). The single strand DNA fragment oligo was used to replace the selection cassette into “pGRAd32 DE1 GAG DE3 ALS” BAC (SEQ ID NO: 61) by recombineering, generating “pGRAd32 DE1 GAG DE3” BAC (SEQ ID NO: 62) This method produced a deletion of the E3 region from bp 28479 to bp 32001 of the GRAd32 wild type genome.

Third Step—Substitution of E4 Region with AmpR-LacZ-SacB Selection Cassette:

The AmpR-LacZ-SacB selection cassette was amplified by PCR using the plasmid “pAmpR-LacZ-SacB” (SEQ ID NO: 39) as template, and the primers below:

(SEQ ID NO: 108)
Fw: 5′-ccc ttc cac ata gct taa att atc acc agt
gca aat gga aaa aaa atc aaa ccc cta ttt gtt tat
ttt tct aa-3′
(SEQ ID NO: 109)
Rev: 5′-cgg cac ttg gcc ttt ttc aca ctc tga tta
gtg ctg gtg ctg tga gag tgt tat ttg tta act gtt
aat tgt cc-3′

The DNA fragment obtained by PCR was then inserted by replacing native GRAd32 E4 region in “pGRAd32 DE1 GAG DE3” (SEQ ID NO: 62) BAC by recombineering, obtaining “pGRAd32 DE1 GAG DE3 DE4 ALS” BAC (SEQ ID NO: 63).

Fourth Step—Deletion of AmpR-LacZ-SacB Selection Cassette for E4 Region Deletion:

The AmpR-LacZ-SacB selection cassette was deleted, and replaced by the human Adenovirus 5 E4orf6, which was amplified by PCR using the genome of purified wild type human Adenovirus 5 (SEQ ID NO: 47) as template, and the primers below:

(SEQ ID NO: 110)
Fw: 5′-ccc ttc cac ata gct taa att atc acc agt
gca aat gga aaa aaa atc aac tac atg ggg gta gag
tca ta-3′
(SEQ ID NO: 111)
Rev: 5′-cgg cac ttg gcc ttt ttc aca ctc tga tta
gtg ctg gtg ctgt gag agt gat gac tac gtc cgg cgt
tcc-3′.

The DNA fragment containing human Ad5 E4 orf6 coding region obtained by PCR was then inserted in “pGRAd32 DE1 GAG DE3 DE4 ALS” BAC (SEQ ID NO: 63) replacing the AmpR-LacZ-SacB selection cassette by recombineering. The final result was “pGRAd32 DE1 GAG DE3 DE4 hAd5E4orf6” BAC (SEQ ID NO: 64). This method produced a deletion of the E4 region from bp 34144 to bp 36821 of the GRAd32 wild type genome.

Construction of GRAd32 DE1DE3DE4 Vector

Substitution of E1 region with AmpR-LacZ-SacB selection cassette:

The AmpR-LacZ-SacB selection cassette was amplified by PCR using the plasmid “pAmpR-LacZ-SacB” (SEQ ID NO: 39) as template, and the primers below:

(SEQ ID NO: 112)
Fw: 5′-tta cgt gaa ttt ccg cgt tcc ggg tca aag
tct ccg ttt tta ttg tca ccg tca tac ccc tat ttg
ttt att ttt ct-3′
(SEQ ID NO: 113)
Rev: 5′-gct aga ccc aaa ctc ggc cct ggt gca ggc
cag cac cag atg gtc agg cct aag ctt att tgt taa
ctg tta att gtc-3′.

The DNA fragment obtained by PCR was then inserted by replacing the CMV::GAG-bGHpA cassette in pGRAd32 DE1 GAG DE3 DE4 hAd5E4orf6″ BAC (SEQ ID NO: 64) BAC by recombineering, obtaining “pGRAd32 DE1 ALS DE3 DE4 hAd5E4orf6” BAC (SEQ ID NO: 26).

### Example 5: Generation of the pGRAd32 DE1 SARS-COV2 DE3DE4 Vector

The full hCMVtetO-IntronA::kozak-SARS CoV-2 S-HA-WPRE-bGHpA cassette was amplified by PCR using the “phCMVtetO-IntronA::SARS CoV-2 S-WPRE-bGHpA” (SEQ ID NO: 54) as template, and the primers below:

(SEQ ID NO: 114)
Fw: 5′-tta cgt gaa ttt ccg cgt tcc ggg tca aag
tct ccg ttt tta ttg tcg ccg tca tct gac ggg ccg
cca tag agc cca ccg cat ccc cag cat gcc tgc tat
t-3′
(SEQ ID NO: 115)
Rev: 5′-gct aga ccc aaa ctc ggc cct ggt gca ggc
cag cac cag atg gtc agg cct aag cgc gat cgc ggc
ccg gcc att gca tac gtt gta tc-3′.

This PCR was cloned into the “pGRAd32 DE1 ALS DE3 DE4 hAd5E4orf6” (SEQ ID NO: 26) previously digested with HpaI by homologous recombination in the E. coli strain BJ5138, to obtain “pGRAd32 DE1 SARS-COV2 DE3 DE4” (SEQ ID NO: 31).

### Example 6: Immunogenicity of GRAd23 DE1 Gag

GRAd23 DE1 expressing the HIV-1 Gag antigen under the control of the tet operator (tetO) was rescued by transfecting GRAd23 DE1 Gag DNA (SEQ ID NO: 41) into a HEK 293-derived packaging cell line expressing the Tet repressor and then amplified by serial passaging following standard procedures. Purified virus was injected in mice in parallel with human Ad5 vector expressing the HIV-1 Gag antigen.

To evaluate T cell response against Gag antigen, groups of six mice were injected with 1×10\hat{ }⁶ and with 1×10\hat{ }⁷ vp/mouse. T cell response was evaluated 3 weeks post-immunization on splenocytes by an ex vivo Interferon-γ enzyme-linked immunospot (Elispot) assay using a HIV Gag peptide T cell epitope mapped in BALB/c mice.

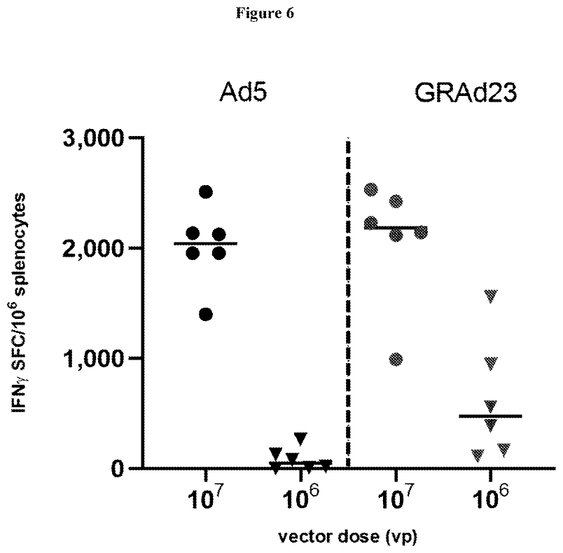

The results are shown in FIG. 6, expressed as IFN-gamma Spot Forming Cells (SFC) per million of splenocytes. Each dot represents the response in a single mouse, and the line corresponds to the mean for each dose group. Injected dose in number of virus particles are shown on the x axis. The results demonstrate a higher immunological potency of the GRAd23 vector compared to the benchmark human Ad5 vector.

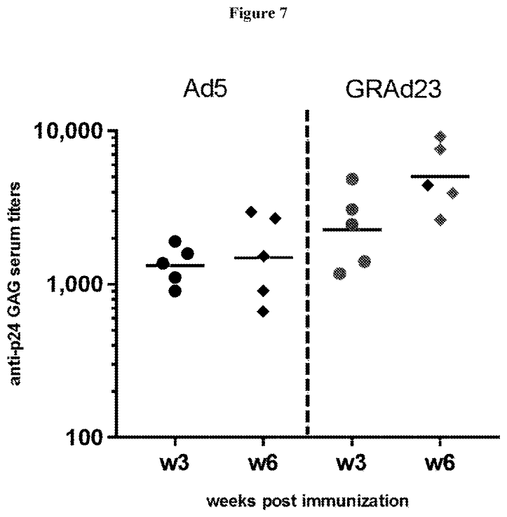

To evaluate the B cell response against the HIV-1 Gag antigen, groups of 5 mice were vaccinated by intramuscularly injecting 5×10\hat{ }⁸ viral particles per mouse of Ad5 or GRAd23 expressing HIV-Gag antigen. The B-cell response was measured at 3 and 6 weeks after the immunization by measuring the antibody response against HIV-1 Gag by ELISA. The results are shown in FIG. 7 and demonstrate a higher antibody titer in mice for the GRAd23 vector compared to the benchmark human Ad5 vector. Each dot represents the response in a single mouse, and the line corresponds to the mean for each dose group.

### Example 7: GRAd23 and GRAd32 Seroprevalence in Humans

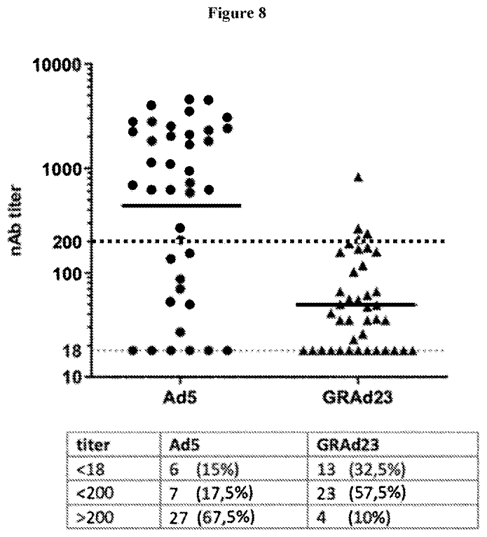

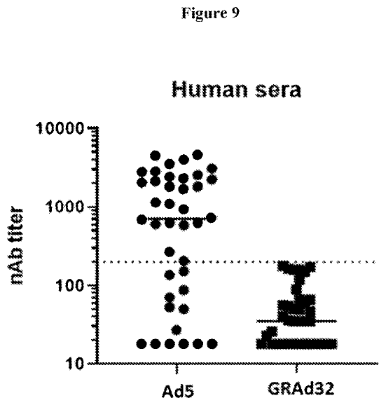

The assay evaluated the effects of neutralising antibody titres from human sera (40 samples) on the ability of human Ad5, gorilla GRAd23 (FIG. 8) or gorilla GRAd32 (FIG. 9) carrying the gene for secreted alkaline phosphatase (SEAP) to transduce HEK 293 cells. SEAP expression in the supernatant of infected cells is revealed by a colorimetric assay. The neutralization titre is defined as the dilution of human serum giving a 50% reduction of the SEAP activity observed in the positive control with the virus alone. The results demonstrate low seroprevalence for both GRAd23 (FIG. 8) and GRAd32 (FIG. 9). The percentage of clinically relevant neutralizing titres (titres >200 which have a negative impact on the vaccination efficiency in humans) is 67.5% for Ad5, while being only 10% for GRAd23 and 0% for GRAd32.

### Example 8: GRAd21 Vector Construction

GRAd21 wt genomic DNA (SEQ ID NO: 10) was isolated by Proteinase K digestion followed by phenol/chloroform extraction and then inserted in pDE1 GRAd shuttle (SEQ ID NO: 40) by homologous recombination in the E. coli strain BJ5138 to obtain the pGRAd21 vector. Homologous recombination between the pIX gene, the right ITR DNA sequences present at the ends of shuttle (digested with I-SceI) and the viral genomic DNA allowed its insertion in the shuttle vector, by deleting at the same time the E1 region that was substituted by the expression cassette, finally generating the “pGRAd21 DE1 GAG” BAC vector (SEQ ID NO: 65).

Construction of GRAd21 DE1 GAG DE3DE4

The construction strategy was based on four different steps as described below:

First Step—Substitution of E3 Region with AmpR-LacZ-SacB Selection Cassette:

The AmpR-LacZ-SacB selection cassette was amplified by PCR using the plasmid “pAmp-LacZ-SacB” (SEQ ID NO: 39) as template, and the primers below:

(SEQ ID NO: 98)
Fw: 5′-ctg tca ttt gtg tgc tga gta taa taa agg
ctg aga tca gaa tct act cga ccc cta ttt gtt tat
ttt tct aa-3′
(SEQ ID NO: 99)
Rev: 5′-agt gat ttt tta ttg att aca gtt atg atc
aat tga aag gga taa ggt ctt att tgt taa ctg tta
att gtc c-3′.

The DNA fragment obtained by PCR was then inserted in “pGRAd21 DE1 GAG” BAC (SEQ ID NO:65) by recombineering, obtaining “pGRAd21 DE1 GAG DE3 ALS” BAC (SEQ ID NO: 66).

Second Step—E3 Region Deletion:

The AmpR-LacZ-SacB selection cassette was deleted using the single strand oligonucleotide 5′-ctg tca ttt gtg tgc tga gta taa taa agg ctg aga tca gaa tct act cgg acc tta tcc ctt tca att gat cat aac tgt aat caa taa aaa atc act-3′ (SEQ ID NO: 86). The single strand DNA fragment oligo was used to replace the selection cassette into “pGRAd21 DE1 GAG DE3 ALS” BAC (SEQ ID NO: 66) by recombineering, generating “pGRAd21 DE1 GAG DE3” BAC (SEQ ID NO: 67) This method produced a deletion of the E3 region from bp 28343 to bp 31875 of the GRAd21 wild type genome.

Third Step—Substitution of E4 Region with AmpR-LacZ-SacB Selection Cassette:

The AmpR-LacZ-SacB selection cassette was amplified by PCR using the plasmid “pAmpR-LacZ-SacB” (SEQ ID NO: 39) as template, and the primers below:

(SEQ ID NO: 87)
Fw: 5′-ccc ttc cac ata gct taa att atc acc agt
gca aat gga aaa aaa atc aaa ccc cta ttt gtt
tat ttt tct aa-3′
(SEQ ID NO: 88)
Rev: 5′-cgg cac ttg gcc ttt ttc aca ctc tga tta
gtg ctg gtg ctg tga gag tgt tat ttg tta act gtt
aat tgt cc-3′

The DNA fragment obtained by PCR was then inserted by replacing native GRAd21 E4 region in “pGRAd21 DE1 GAG DE3” (SEQ ID NO: 67) BAC by recombineering, obtaining “pGRAd21 DE1 GAG DE3 DE4 ALS” BAC (SEQ ID NO: 68).

Fourth Step—Deletion of AmpR-LacZ-SacB Selection Cassette for E4 Region Deletion:

The AmpR-LacZ-SacB selection cassette was deleted, and replaced by the human Adenovirus 5 E4orf6, which was amplified by PCR using the genome of purified wild type human Adenovirus 5 (SEQ ID NO: 47) as template, and the primers below:

(SEQ ID NO: 89)
Fw: 5′-ccc ttc cac ata gct taa att atc acc agt
gca aat gga aaa aaa atc aac taca tgg ggg tag
agt cat a-3′
(SEQ ID NO: 90)
Rev: 5′-cgg cac ttg gcc ttt ttc aca ctc tga tta
gtg ctg gtg ctgt gag agt gat gac tac gtc cgg
cgt tcc-3′.

The DNA fragment containing human Ad5 E4 orf6 coding region obtained by PCR was then inserted in “pGRAd21 DE1 GAG DE3 DE4 ALS” BAC (SEQ ID NO: 68) replacing the AmpR-LacZ-SacB selection cassette by recombineering. The final result was “pGRAd21 DE1 GAG DE3 DE4 hAd5E4orf6” BAC (SEQ ID NO: 69) This method produced a deletion of the E4 region from bp 34005 to bp 36681 of the GRAd21 wild type genome.

Construction of GRAd21DE1DE3DE4 Empty Vector

Substitution of E1 Region with AmpR-LacZ-SacB Selection Cassette:

The AmpR-LacZ-SacB selection cassette was amplified by PCR using the plasmid “pAmpR-LacZ-SacB” (SEQ ID NO: 39) as template, and the primers below:

(SEQ ID NO: 112)
Fw: 5′-tta cgt gaa ttt ccg cgt tcc ggg tca aag
tct ccg ttt tta ttg tca ccg tca tac ccc tat ttg
ttt att ttt ct-3′
(SEQ ID NO: 113)
Rev: 5′- gct aga ccc aaa ctc ggc cct ggt gca
ggc cag cac cag atg gtc agg cct aag ctt att
tgt taa ctg tta att gtc-3′.

The DNA fragment obtained by PCR was then inserted by replacing the CMV::GAG-bGHpA cassette in pGRAd21 DE1 GAG DE3 DE4 hAd5E4orf6″ BAC (SEQ ID NO: 68) BAC by recombineerin6, obtaining “pGRAd21 DE1 ALS DE3 DE4 hAd5E4orf6” BAC (SEQ ID NO: 28).

Generation of the pGRAd21 DE1 SARS-COV2 DE3 DE4 hAd5E4orf6 vector

The full hCMVtetO-IntronA::kozak-SARS CoV-2 S-HA-WPRE-bGHpA cassette was amplified by PCR using the “phCMVtetO-IntronA::SARS CoV-2 S-WPRE-bGHpA” (SEQ ID NO: 55) as template, and the primers below:

(SEQ ID NO: 116)
Fw: 5′-acc caa act cgg ccc tgg tgc agg cca gca
cca gat ggt cag gcc taa gcg aca ttg att att gac
tag tta tta-3′
(SEQ ID NO: 117)
Rev: 5′-tcc gcg ttc cgg gtc aaa gtc tcc gtt ttt
att gtc gcc gtc atc tga cgt ccc cag cat gcc tgc
tat t-3′.

This PCR was cloned into the “pGRAd21 DE1 ALS DE3 DE4 hAd5E4orf6” (SEQ ID NO: 28) by recombineering in the E. coli strain SW102, to obtain “pGRAd21 DE1 SARS-COV2 DE3 DE4” (SEQ ID NO: 33).

### Example 9: Construction of GRAd33, GRAd34, GRAd35, GRAd36, GRAd37 and GRAd38 Vectors

GRAd33, GRAd34, GRAd35, GRAd36, and GRAd38 vector constructs were constructed by inserting the following segments of the GRAd33, GRAd34, GRAd35, GRAd36 and GRAd38 hexon into GRAd23-derived target vector constructs by standard homologous recombination:

GRAd33 recombination segment: nucleotides 19381 to 21586 of SEQ ID NO: 16

GRAd34 recombination segment: nucleotides 19381 to 20491 of SEQ ID NO: 20

GRAd35 recombination segment: nucleotides 19381 to 20491 of SEQ ID NO: 18

GRAd36 recombination segment: nucleotides 19381 to 21591 of SEQ ID NO: 5

GRAd38 recombination segment: nucleotides 19381 to 20491 of SEQ ID NO: 8

The GRAd37 vector constructs were constructed by inserting the following segment of

the GRAd37 fiber into GRAd21-derived target vector constructs by standard homologous recombination:

GRAd37 recombination segment: nucleotides 33189 to 33779 of SEQ ID NO: 14

### Example 10: Immunogenicity of GRAd21 DE1 Gag

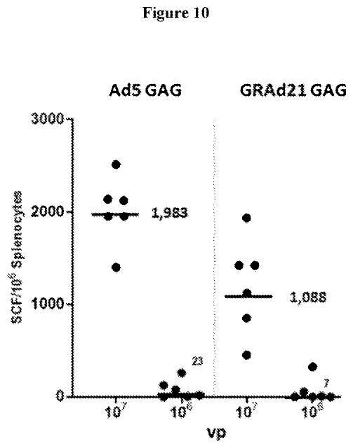

Immunogenicity of the GRAd21 gorilla vectors in comparison with human Ad5. Balb/c mice were immunised with 10⁶ and 10⁷ viral particles (VP) of hAd5 or GRAd21 vectors encoding the HIV-1 gag protein. After 21 days post prime, spleen was collected and T cell responses were measured by IFNg-ELISpot after stimulation with gag peptides. Horizontal bars indicate the mean value. Immunogenicity of GRAd21 was comparable to that observed for human Ad5 (FIG. 10).

### Example 11: Expression and Immunogenicity of GRAd32 DE1 Spike

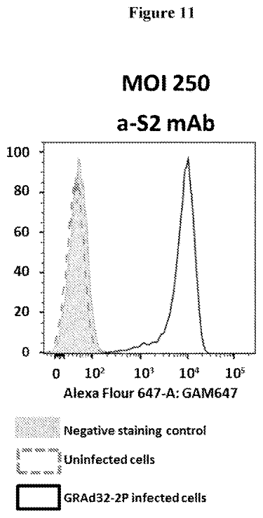

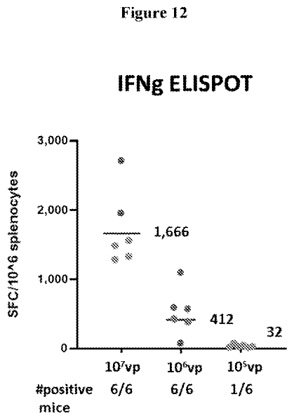

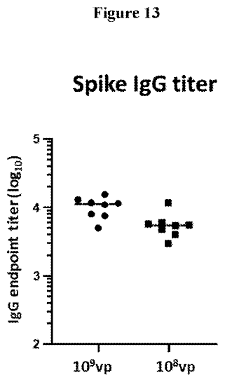

Expression and immunogenicity of GRAd32 DE1 encoding the SARS-COV2 Spike antigen (GRAd32-S). FIG. 11: Whole-cell FACS analysis of HeLa cells infected with GRAd32-S at MOI=250. 48 hours after infection cells were detached and stained with the anti-S2 polyclonal antibody (40590-T62) from SinoBiologicals. FIG. 12: IFN-γ spleen ELISpot responses after immunisation with 10⁷, 10⁶ or 10⁵ VP. Balb/c mice were immunised intramuscularly and T cell responses to a peptide pool spanning the full length S protein were assayed two weeks post immunisation. FIG. 13: Serum antibody responses against the Spike antigen following immunization with GRAd32-S in Balb/c mice were measured by ELISA on spike-coated 96-well plates. Data are expressed as IgG endpoint titres of individual sera from animals immunised with 10⁹ and 10⁸ VP of GRAd32-S, five weeks after immunization.

### Example 12: In Vitro Expression of SARS CoV2 Spike Using Different GRAd Vectors

Antigen expression of vectors GRAd23b-S2P, GRAd32b-S2P, GRAd34b-S2P and GRAd39b-S2P encoding the prototype SARS CoV2 Spike protein stabilized in its pre-fusion conformation (S2P). For all these vectors “b” indicates that both the E1 and E3 regions have been deleted in the respective viral genomes.

GRAD32b-S2P was generated by replacing, through standard homologous recombination, GAG in “pGRAd32 DE1 GAG DE3” (SEQ ID NO: 62) with a modified version of SARS CoV2 Spike protein (SEQ ID NO:29) stabilized in its pre-fusion conformation by substituting codons for Lys986 and Va1987 into Pro. GRAd39b-S2P was then generated by replacing, through standard homologous recombination, the hexon encoding region of GRAD32b S2P with the GRAd34 hexon (nucleotides 19381 to 20491 of SEQ ID NO: 20). GRAD23b S2P was analogously constructed replacing, through standard homologous recombination, GAG in “pGRAd23 DE1 GAG DE3 BAC” (SEQ ID NO: 45) with the S2P version of the spike protein.

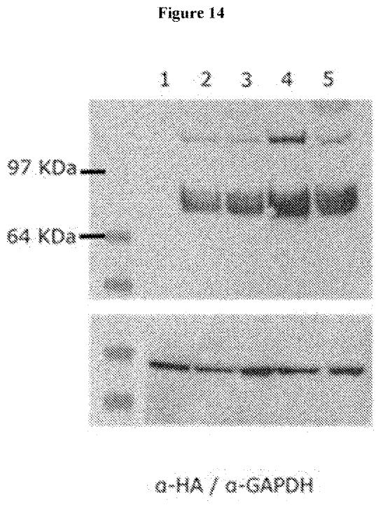

While there was no statistically significant difference in the productivity level of these vectors (viral particles produced per cell at a given time point after start of synchronous infection, data not shown), expression of the spike antigen showed an unexpected increase for one of the GRAd vectors. HeLa cells were infected with 50 MOI of each vector, and collected cell lysates 48 hours after infection. Western blot analysis revealed that the level of antigen produced by the cells were higher in samples infected with GRAd34b-S2P (FIG. 14).

### Example 13: In Vivo Expression of SARS CoV2 Spike Using Different GRAd Vectors

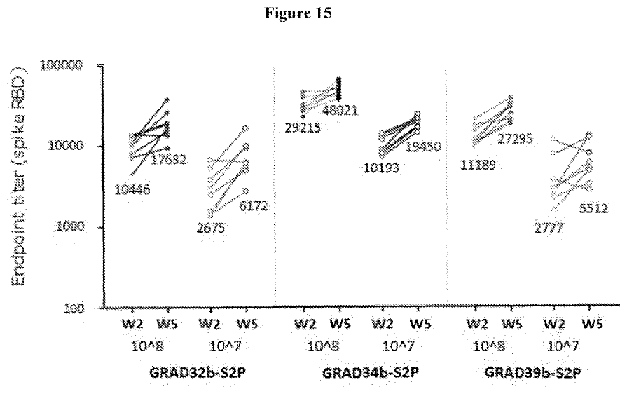

GRAd32b-S2P, GRAd34b-S2P and GRAd39b-S2P were further tested in immunogenicity experiments in mice. Wild type BALB/c mice were infected with 10 1 \8 or 10 1 \7 viral particles of GRAd32b-S2P, GRAd34b-S2P or GRAd39b-S2P and sera were collected 2 or 5 weeks post vaccination. FIG. 15 shows the endpoint titer of antibodies generated against the Spike-2P antigen, as measured by ELISA on a recombinant spike receptor-binding-domain (RBD) protein. Also in this case, GRAD34b-S2P showed a clear about 2- to 3-fold improvement relative to GRAD32b-S2P, also at the lower dose.

### Example 14: Clinical Trial of a GRAd Vector Expressing SARS CoV2 Spike

GRAD32b-S2P expressing the SARS-COV2 spike protein stabilized by the two Pro mutations (in the following called GRAd-COV2, sequence according to SEQ ID NO: 31 but with the substitutions Pos 2487 C->T, Pos 2488 A->G, Pos 2489 C->G, Pos 2490 C->A, Pos 2491 T->G, Pos 2492 T->G) resulting in a spike protein according to SEQ ID NO: 25) was then subjected to a dose-escalation, open label clinical trial designed to determine its safety and immunogenicity. The study included two age cohorts, of either younger (18-55) or older (65-adults. Each cohort consisted of 3 arms of 15 volunteers each, for assessing a single administration at three different dose levels of GRAd-COV2: low dose (LD) 5×10\hat{ }10; intermediate dose (ID) 1×10\hat{ }11 and high dose (HD) 2×10\hat{ }11 viral particles (vp). Safety and immunogenicity endpoints were collected in the first 4 weeks after vaccination for volunteers enrolled in both age cohorts. GRAd-COV2 was manufactured under good manufacturing practice conditions and suspended in formulation buffer at a concentration of 2×10\hat{ }11 vp/mL. Volunteers received a single intramuscular injection in the deltoid. For administration of the HD, 1 ml of GRAd-COV2 was injected without dilution. For ID and LD, the vaccine was diluted in sterile saline solution to reach a final 1 ml injection volume. As comparator for immunogenicity analysis, three independent sets of anonymized specimens (sera and PBMC) from COVID-19 patients either hospitalized or recovering from mild symptomatic disease, collected 20 to 60 days after symptom onset, were used. A research reagent for anti-SARS-CoV-2 Ab (NIBSC code 20/130), a human plasma from a donor recovered from COVID-19, was included as a positive control.

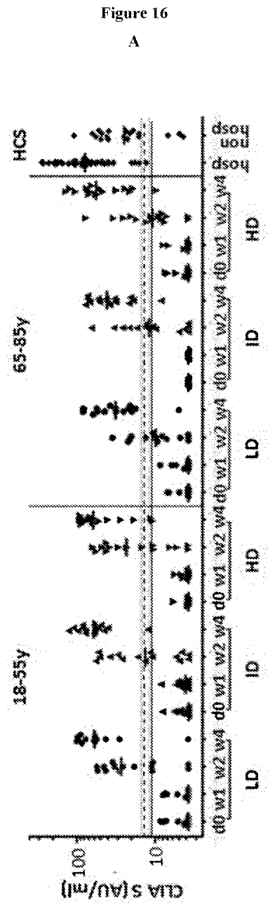

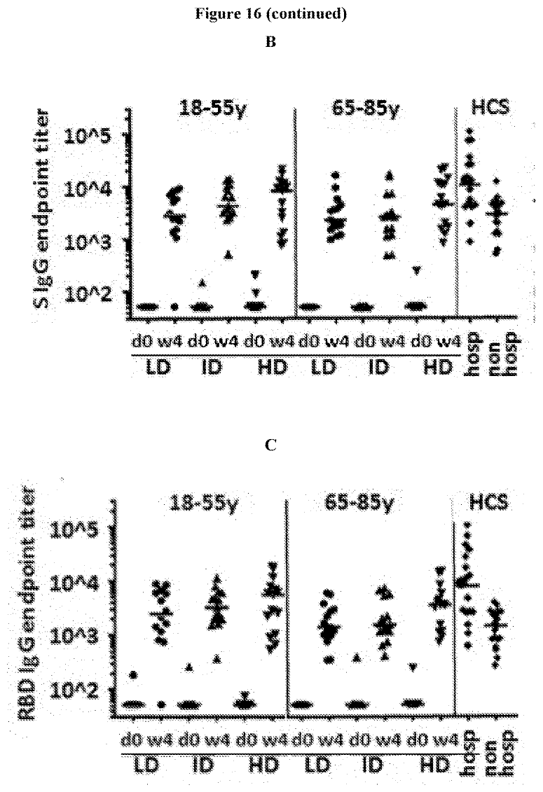

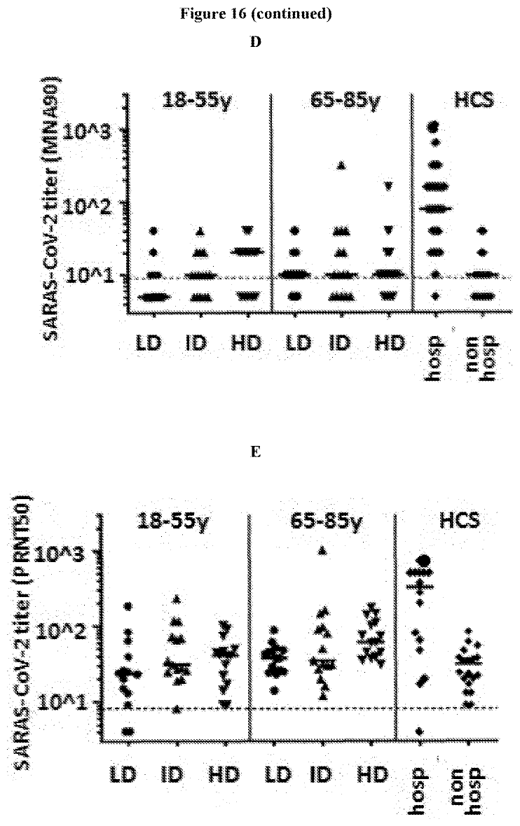

Antibody response to GRAd-COV2 vaccination was monitored by a clinically validated chemiluminescence immunoassay (CLIA), revealing similar kinetics of anti-S IgG induction in all study groups (FIG. 16A). Importantly, high vaccine dose provided similar lever of IgG among both age cohorts after 4 weeks following vaccination (high dose arms median IgG was 61.8 in younger and 56.3 in older adults). The ELISA assay showed that 89 (98.8%) out of the volunteers had developed detectable level of anti-S IgG (including both antibodies against the whole Spike protein and antibodies specific for the RBD) (FIG. 16B-C).

Neutralizing antibodies to SARS-CoV-2 were assessed by two different in vitro assays, both using SARS-CoV-2 live virus. Microneutralization assay (MNA90) at week 4 after vaccination detected neutralizing antibodies in the serum of 25/44 (56.8%) young volunteers and 33/45 (73.3%) older age volunteers (FIG. 16D). Plaque reduction neutralization test (PRNT50) revealed that SARS-CoV-2 neutralizing antibodies were detectable in 42/44 (92.5%) younger and in 45/45 (100%) older age volunteers (FIG. 16E). Across all arms, titers of binding and neutralizing antibodies elicited by GRAd-COV2 vaccination were in the range of those measured in subjects recovered from mild COVID-19 (FIG. 16A-D).

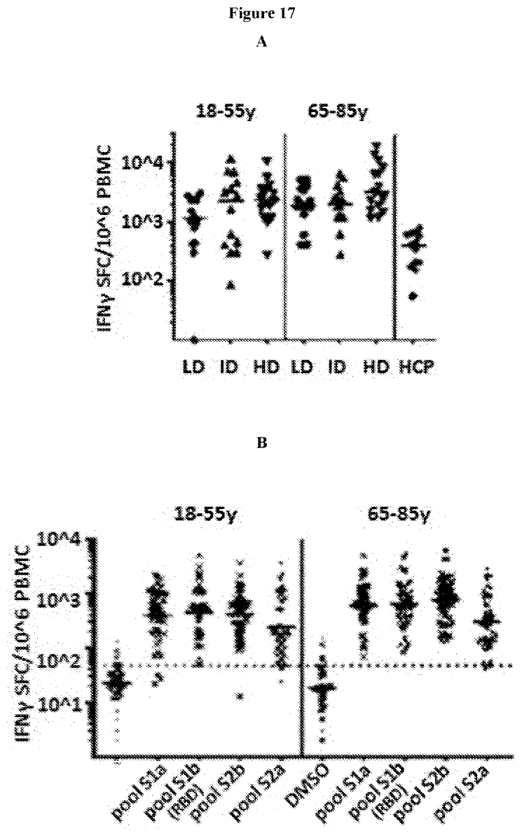

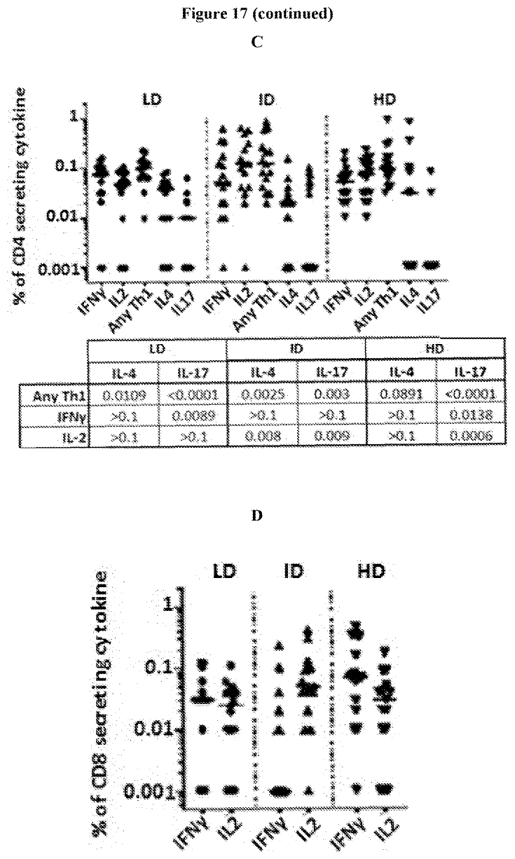

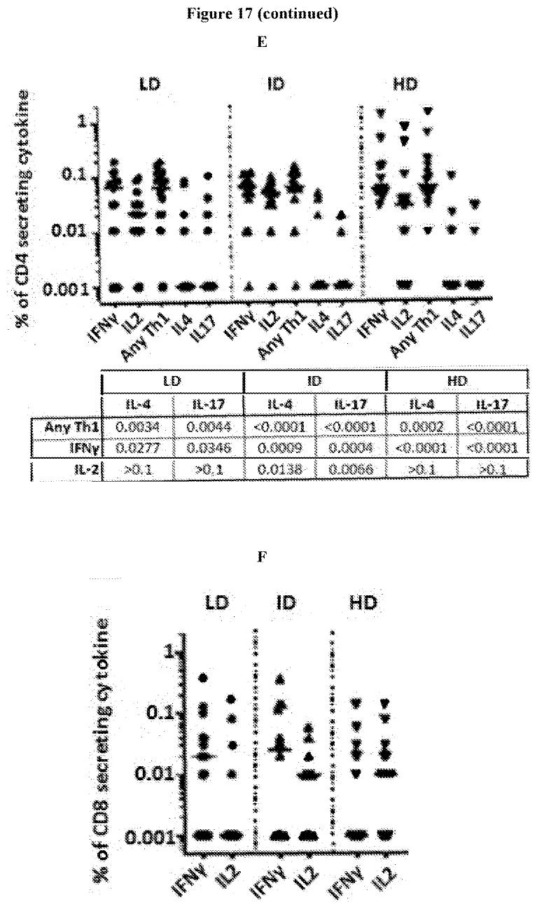

A quantitative IFNγ ELISpot assay was then used to assess T cell response on freshly isolated PBMCs from volunteers in both cohorts at week 2 after vaccination. GRAd-COV2 administration at all three doses induced potent S-specific IFNγ producing T cell response in both cohorts (FIG. 17A), with 80% of evaluable subjects across the two age cohorts showed a response above 1000 SFC/million PBMC. No significant differences between younger and older study arms receiving the same vaccine dose (p was 0.116, 0.984 and 0.152 for LD, ID and HD, respectively). All regions of the S protein had a similar degree of immunogenicity in both age cohorts (FIG. 17B). S-specific T cell response was generally higher in GRAd-COV2 vaccinated subjects than in SARS-CoV-2 convalescent controls who were sampled 1-2 months after symptoms onset. Intracellular staining for cytokine production (ICS) and FACS analysis revealed that vaccine induced responses involved both S protein specific CD4 and CD8 T lymphocytes in younger and older volunteers (FIG. 17C-D and E-F), with a slightly higher S-specific CD4 than CD8 T cell responses. Importantly, among GRAd-COV2 vaccine induced S-specific CD4, IFNγ production was more prominent than IL4 and IL17 in both age cohorts, indicating that the vaccine induced a predominantly T helper 1 (Th1) response (tables below FIGS. 17C and 17E).

Taken together these data show that GRAd-COV2 is an efficient vaccine vector eliciting both antibody and T-cell responses across all age groups.

1. An isolated polynucleotide encoding an adenovirus hexon protein comprising:
A) (i) a HVR1 comprising an amino acid sequence according to position 136 to 168 of SEQ ID NO: 2, or a variant thereof comprising up to two mutations,
(ii) a HVR2 comprising an amino acid sequence according to position 187 to 201 of SEQ ID NO: 2, or a variant thereof comprising up to two mutations,
(iii) a HVR3 comprising an amino acid sequence according to position 219 to 225 of SEQ ID NO: 2, or a variant thereof comprising up to two mutations,
(iv) a HVR4 comprising an amino acid sequence according to position 257 to 268 of SEQ ID NO: 2, or a variant thereof comprising up to two mutations,
(v) a HVR5 comprising an amino acid sequence according to position 276 to 290 of SEQ ID NO: 2, or a variant thereof comprising up to two mutations,
(vi) a HVR6 comprising an amino acid sequence according to position 314 to 322 Y of SEQ ID NO: 2, or a variant thereof comprising up to two mutations, and
(vii) a HVR7 comprising an amino acid sequence according to position 431 to 456 of SEQ ID NO: 2, or a variant thereof comprising up to two mutations; or
B) (i) a HVR1 comprising an amino acid sequence according to position 136 to 168 of SEQ ID NO: 9, or a variant thereof comprising up to two mutations,
(ii) a HVR2 comprising an amino acid sequence according to position 187 to 201 of SEQ ID NO: 9, or a variant thereof comprising up to two mutations,
(iii) a HVR3 comprising an amino acid sequence according to position 219 to 225 of SEQ ID NO: 9, or a variant thereof comprising up to two mutations,
(iv) a HVR4 comprising an amino acid sequence according to position 257 to 268 of SEQ ID NO: 9, or a variant thereof comprising up to two mutations,
(v) a HVR5 comprising an amino acid sequence according to position 276 to 290 of SEQ ID NO: 9, or a variant thereof comprising up to two mutations,
(vi) a HVR6 comprising an amino acid sequence according to position 314 to 322 of SEQ ID NO: 9, or a variant thereof comprising up to two mutations, and
(vii) a HVR7 comprising an amino acid sequence according to position 431 to 456 of SEQ ID NO: 9, or a variant thereof comprising up to two mutations; or
C) (i) a HVR1 comprising an amino acid sequence according to position 136 to 163 of SEQ ID NO: 11, or a variant thereof comprising up to two mutations,
(ii) a HVR2 comprising an amino acid sequence according to position 182 to 196 of SEQ ID NO: 11, or a variant thereof comprising up to two mutations,
(iii) a HVR3 comprising an amino acid sequence according to position 214 to 220 of SEQ ID NO: 11, or a variant thereof comprising up to two mutations,
(iv) a HVR4 comprising an amino acid sequence according to position 252 to 262 of SEQ ID NO: 11, or a variant thereof comprising up to two mutations,
(v) a HVR5 comprising an amino acid sequence according to position 270 to 278 of SEQ ID NO: 11, or a variant thereof comprising up to two mutations,
(vi) a HVR6 comprising an amino acid sequence according to position 302 to 310 of SEQ ID NO: 11, or a variant thereof comprising up to two mutations, and
(vii) a HVR7 comprising an amino acid sequence according to position 419 to 442 of SEQ ID NO: 11, or a variant thereof comprising up to two mutations; or
D) (i) a HVR1 comprising an amino acid sequence according to position 136 to 168 of SEQ ID NO: 17, or a variant thereof comprising up to two mutations,
(ii) a HVR2 comprising an amino acid sequence according to position 187 to 201 of SEQ ID NO: 17, or a variant thereof comprising up to two mutations,
(iii) a HVR3 comprising an amino acid sequence according to position 219 to 225 of SEQ ID NO: 17, or a variant thereof comprising up to two mutations,
(iv) a HVR4 comprising an amino acid sequence according to position 257 to 267 of SEQ ID NO: 17, or a variant thereof comprising up to two mutations,
(v) a HVR5 comprising an amino acid sequence according to position 275 to 289 of SEQ ID NO: 17, or a variant thereof comprising up to two mutations,
(vi) a HVR6 comprising an amino acid sequence according to position 313 to 321 of SEQ ID NO: 17, or a variant thereof comprising up to two mutations, and
(vii) a HVR7 comprising an amino acid sequence according to position 430 to 455 of SEQ ID NO: 17, or a variant thereof comprising up to two mutations; or
E) (i) a HVR1 comprising an amino acid sequence according to position 136 to 168 of SEQ ID NO: 19, or a variant thereof comprising up to two mutations,
(ii) a HVR2 comprising an amino acid sequence according to position 187 to 201 of SEQ ID NO: 19, or a variant thereof comprising up to two mutations,
(iii) a HVR3 comprising an amino acid sequence according to position 219 to 225 of SEQ ID NO: 19, or a variant thereof comprising up to two mutations,
(iv) a HVR4 comprising an amino acid sequence according to position 257 to 268 of SEQ ID NO: 19, or a variant thereof comprising up to two mutations,
(v) a HVR5 comprising an amino acid sequence according to position 276 to 290 of SEQ ID NO: 19, or a variant thereof comprising up to two mutations,
(vi) a HVR6 comprising an amino acid sequence according to position 314 to 322 of SEQ ID NO: 19, or a variant thereof comprising up to two mutations, and
(vii) a HVR7 comprising an amino acid sequence according to position 431 to 456 of SEQ ID NO: 19, or a variant thereof comprising up to two mutations; or
F) (i) a HVR1 comprising an amino acid sequence according to position 136 to 168 of SEQ ID NO: 21, or a variant thereof comprising up to two mutations,
(ii) a HVR2 comprising an amino acid sequence according to position 187 to 201 of SEQ ID NO: 21, or a variant thereof comprising up to two mutations,
(iii) a HVR3 comprising an amino acid sequence according to position 219 to 225 of SEQ ID NO: 21, or a variant thereof comprising up to two mutations,
(iv) a HVR4 comprising an amino acid sequence according to position 257 to 267 of SEQ ID NO: 21, or a variant thereof comprising up to two mutations,
(v) a HVR5 comprising an amino acid sequence according to position 275 to 289 of SEQ ID NO: 21, or a variant thereof comprising up to two mutations,
(vi) a HVR6 comprising an amino acid sequence according to position 313 to 321 of SEQ ID NO: 21, or a variant thereof comprising up to two mutations, and
(vii) a HVR7 comprising an amino acid sequence according to position 430 to 455 of SEQ ID NO: 21, or a variant thereof comprising up to two mutations; or
G) (i) a HVR1 comprising an amino acid sequence according to position 136 to 168 of SEQ ID NO: 23, or a variant thereof comprising up to two mutations,
(ii) a HVR2 comprising an amino acid sequence according to position 187 to 201 of SEQ ID NO: 23, or a variant thereof comprising up to two mutations,
(iii) a HVR3 comprising an amino acid sequence according to position 219 to 225 of SEQ ID NO: 23, or a variant thereof comprising up to two mutations,
(iv) a HVR4 comprising an amino acid sequence according to position 257 to 268 of SEQ ID NO: 23, or a variant thereof comprising up to two mutations,
(v) a HVR5 comprising an amino acid sequence according to position 276 to 290 of SEQ ID NO: 23, or a variant thereof comprising up to two mutations,
(vi) a HVR6 comprising an amino acid sequence according to position 314 to 322 of SEQ ID NO: 23, or a variant thereof comprising up to two mutations, and
(vii) a HVR7 comprising an amino acid sequence according to position 431 to 456 of SEQ ID NO: 23, or a variant thereof comprising up to two mutations; wherein the polynucleotide encoding an adenovirus hexon protein according to G) further encodes for an adenovirus fiber protein according to SEQ ID NO: 6, or a variant thereof comprising up to two mutations.

2. The isolated polynucleotide of claim 1, wherein the hexon protein according to
A) comprises an amino acid sequence according to SEQ ID NO: 2, or a variant thereof having at least 80% sequence identity,
B) comprises an amino acid sequence according to SEQ ID NO: 9, or a variant thereof having at least 80% sequence identity,
C) comprises an amino acid sequence according to SEQ ID NO: 11, or a variant thereof having at least 80% sequence identity,
D) comprises an amino acid sequence according to SEQ ID NO: 17, or a variant thereof having at least 80% sequence identity, and/or
E) comprises an amino acid sequence according to SEQ ID NO: 19, or a variant thereof having at least 80% sequence identity,
F) comprises an amino acid sequence according to SEQ ID NO: 21, or a variant thereof having at least 80% sequence identity, and/or
G) comprises an amino acid sequence according to SEQ ID NO: 23, or a variant thereof having at least 80% sequence identity.

3. The isolated polynucleotide of claim 1, further encoding an adenoviral fiber protein comprising with respect to
A) an amino acid sequence according to SEQ ID NO: 3 or SEQ ID NO: 6, or a variant thereof having at least 80% sequence identity,
B), D), E) and/or F) an amino acid sequence according to SEQ ID NO: 6, or a variant thereof having at least 80% sequence identity, and/or
C) an amino acid sequence according to SEQ ID NO: 12 or SEQ ID NO: 15, or a variant thereof having at least 80% sequence identity.

4. The isolated polynucleotide of claim 1, further encoding an adenoviral penton protein comprising with respect to
A) an amino acid sequence according to SEQ ID NO: 4 or SEQ ID NO: 7, or a variant thereof having at least 80% sequence identity,
B), D), E), F) and/or G) an amino acid sequence according to SEQ ID NO: 7, or a variant thereof having at least 80% sequence identity, and/or
C) an amino acid sequence according to SEQ ID NO: 13, or a variant thereof having at least 80% sequence identity.

5. The isolated polynucleotide of claim 1, wherein the adenovirus comprises a non-adenoviral gene, protein or fragment thereof, and wherein the non-adenoviral gene or protein optionally is a coronaviral gene or protein, preferably a SARS-CoV-2 gene or protein.

6. The isolated polynucleotide of claim 5, wherein the non-adenoviral gene or protein is a coronaviral gene or protein, and wherein the coronaviral gene or protein is a spike gene or protein, preferably comprising a sequence according to SEQ ID NO: 30 or a variant thereof having at least 80% amino acid sequence identity.

7. An isolated hexon polypeptide encoded by the polynucleotide as defined in claim 1 A), B), C), D), E) or F).

8. An isolated adenoviral capsid comprising hexon and preferably also fiber and/or penton proteins encoded by the polynucleotide of claim 1.

9. An adenovirus (i) encoded by a polynucleotide of claim 1, (ii) comprising a polynucleotide of claim 1 and/or (iii) comprising a hexon polypeptide of encoded by the polynucleotide as defined in claim 1 A), B), C), D), E) or E) or an adenoviral capsid comprising hexon and preferably also fiber and/or penton proteins encoded by the polynucleotide of claim 1.

10. A virus-like particle encoded by a polynucleotide of claim 1.

11. A vector comprising a polynucleotide of claim 1.

12. A composition comprising (i) an adjuvant, (ii) a polynucleotide of claim 1, a hexon polypeptide encoded by the polynucleotide as defined in claim 1 A), B), C), D), E) or F), an adenoviral capsid comprising hexon and preferably also fiber and/or penton proteins encoded by the polynucleotide of claim 1, an adenovirus, virus-like particle, or vector encoded of by the polynucleotide of claim 1, and optionally (iii) a pharmaceutically acceptable excipient.

13. An isolated cell comprising a polynucleotide of claim 1, a hexon polypeptide encoded by the polynucleotide as defined in claim 1 A), B), C), D), E) or F), an adenoviral capsid comprising hexon and preferably also fiber and/or Denton proteins encoded by the polynucleotide of claim 1, an adenovirus-, virus-like particle, or vector encoded by the polynucleotide of claim 1.

14. A method for treating or preventing a disease, wherein the disease is a coronavirus disease, comprising administering an effective amount of the polynucleotide of claim 1, a hexon polypeptide encoded by the polynucleotide as defined in claim 1 A), B), C), D), E) or F) of claim 7, an adenoviral capsid comprising hexon and preferably also fiber and/or penton proteins encoded by the polynucleotide of claim 1, an adenovirus, virus-like particle, composition, and/or isolated cell or vector encoded by the polynucleotide of claim 1.

15. An in vitro method for producing an adenovirus or an adenovirus-like particle, comprising the steps of
(i) expressing a polynucleotide of claim 1 in a cell such that an adenovirus or an adenovirus-like particle is assembled in the cell,
(ii) isolating the adenovirus or the adenovirus-like particle from the cell or the medium surrounding the cell.

16. An isolated hexon polypeptide encoded by the polynucleotide as defined in claim 2 A), B), C), D), E) or F).
